SEQUENCE LISTING (352 sequences)

SEQ ID NO: 1 - DNA, 50 bp, Artificial sequence
  Synthesized
tcatcatcttcactagaaatctcctcacttcccgcattgggctttgtccc

SEQ ID NO: 2 - DNA, 48 bp, Artificial sequence
  Synthesized
ctctttgcagggacccttcttcgttgctgcttcttctcttctaccagc

SEQ ID NO: 3 - DNA, 38 bp, Artificial sequence
  Synthesized
gggccccagcgcactgttcttggccacaccaatggtgg

SEQ ID NO: 4 - DNA, 50 bp, Artificial sequence
  Synthesized
acgaagaggcacacgagggtgatgaccagccacgaggcccgcagccgccg

SEQ ID NO: 5 - DNA, 35 bp, Artificial sequence
  Synthesized
caaggcatggcgtgcagggcagtctctctggaggg

SEQ ID NO: 6 - DNA, 50 bp, Artificial sequence
  Synthesized
ggcaggtacatctagcacaatcacagtcctgtcacactgccaacgtggcc

SEQ ID NO: 7 - DNA, 50 bp, Artificial sequence
  Synthesized
tgcctcatcaacacgtcaccaccctttgctcttcttccaattagtcacat

SEQ ID NO: 8 - DNA, 50 bp, Artificial sequence
  Synthesized
tgccacagcctttcacgtttcctaaaccctagtaacctctgatctccatc

SEQ ID NO: 9 - DNA, 50 bp, Artificial sequence
  Synthesized
agtgttccagaaatgatgtccatttttggcatgactccatcccgatcccc

SEQ ID NO: 10 - DNA, 50 bp, Artificial sequence
  Synthesized
gagaactcgtgctggcgatgtaccctccaacacaaccaaaggctttgcca

SEQ ID NO: 11 - DNA, 50 bp, Artificial sequence
  Synthesized
tggaatctaccccttcctcacatgcctttaggaagttgagtccagggaag

SEQ ID NO: 12 - DNA, 50 bp, Artificial sequence
  Synthesized
caacatccattctctactccctctactctgatggcacccggattagattg

SEQ ID NO: 13 - DNA, 35 bp, Artificial sequence
  Synthesized
ttggcctggtaggtctcggggatgagtacggagcg

SEQ ID NO: 14 - DNA, 35 bp, Artificial sequence
  Synthesized
cggactgaggccagccgcacacgccctggcaattg

SEQ ID NO: 15 - DNA, 50 bp, Artificial sequence
  Synthesized
tttataggatagttcttcctctggtgtaatatcctggagctcctcttgca

SEQ ID NO: 16 - DNA, 50 bp, Artificial sequence
  Synthesized
atgctttggtgcctagtgatgaaccgcttggaaagtgccagcccattggt

SEQ ID NO: 17 - DNA, 47 bp, Artificial sequence
  Synthesized
gtaatgctgatgatggtagaggtggcgtcctgcttccggattaagtc

SEQ ID NO: 18 - DNA, 50 bp, Artificial sequence
  Synthesized
agttgctctggacaaagtcccagaccagaccttgcccaatgacgttgttg

SEQ ID NO: 19 - DNA, 46 bp, Artificial sequence
  Synthesized
cggaggggcactctgaatccttgctggagggcttggttgggaggtc

SEQ ID NO: 20 - DNA, 50 bp, Artificial sequence
  Synthesized
cagaaccaccaccaggaccgggagcgacaggaagagcctcatggcgaggc

SEQ ID NO: 21 - DNA, 50 bp, Artificial sequence
  Synthesized
tttgaagagaggggttggctggcttcttctcctggagaagcagaaatctg

SEQ ID NO: 22 - DNA, 50 bp, Artificial sequence
  Synthesized
cagtaaggctagatgtaagagggaaagtcggactgtagtctctcagtgtg

SEQ ID NO: 23 - DNA, 50 bp, Artificial sequence
  Synthesized
gacttgtgcatgcggtactcattgaaaaccagatcaggggcgaagtagag

SEQ ID NO: 24 - DNA, 50 bp, Artificial sequence
  Synthesized
caaactcttgagagaggtgcctcattcggacacactggctgtacatccgg

SEQ ID NO: 25 - DNA, 38 bp, Artificial sequence
  Synthesized
cagcgcttgggcacaaagcacatgacctccacagcttg

SEQ ID NO: 26 - DNA, 40 bp, Artificial sequence
  Synthesized
ctcaaatccccgcaatctccccagcgtcatcatatcgttg

SEQ ID NO: 27 - DNA, 50 bp, Artificial sequence
  Synthesized
aaggaaattgctgagtcacgagaacacgttttggacctccaactggagct

SEQ ID NO: 28 - DNA, 50 bp, Artificial sequence
  Synthesized
acacaagacccgctgagcctggccactatttacaggtaatggattctgac

SEQ ID NO: 29 - DNA, 38 bp, Artificial sequence
  Synthesized
cacggagcgagacatctcggcccgaatgctgtcagctt

SEQ ID NO: 30 - DNA, 39 bp, Artificial sequence
  Synthesized
caggccagaaagagagagtagcgcgagcacagctaaggc

SEQ ID NO: 31 - DNA, 50 bp, Artificial sequence
  Synthesized
tccctcgccgggtggatgaaaccaaaaatacggagtccatagttcttcca

SEQ ID NO: 32 - DNA, 48 bp, Artificial sequence
  Synthesized
cagaactccgagttgtcgtctgaggccacagaaaactggacgtctccg

SEQ ID NO: 33 - DNA, 50 bp, Artificial sequence
  Synthesized
agtgctttctttagactgtctcggactgtaactccacaccttgcaggtac

SEQ ID NO: 34 - DNA, 50 bp, Artificial sequence
  Synthesized
cctgaattctgtaaacagcacagcactctgggattagacctctcatcatc

SEQ ID NO: 35 - DNA, 48 bp, Artificial sequence
  Synthesized
caaggaatacatgcaaggctgactagccagccatcatcccaaggagag

SEQ ID NO: 36 - DNA, 50 bp, Artificial sequence
  Synthesized
acaagaataccaagtagtcttgcagaacatggggcactctcccattcagc

SEQ ID NO: 37 - DNA, 50 bp, Artificial sequence
  Synthesized
gtacttctgggctttacttgaatctaggccggcaactgccatgatctgtt

SEQ ID NO: 38 - DNA, 44 bp, Artificial sequence
  Synthesized
gttgctggaggggtggcccacgaccgggtcgagtgactcggtga

SEQ ID NO: 39 - DNA, 50 bp, Artificial sequence
  Synthesized
ttgaggcttatccattcggacagcaagtttgattttgagatcttggtcgg

SEQ ID NO: 40 - DNA, 50 bp, Artificial sequence
  Synthesized
ttccagacattcttaccgatggcccataaatacccagaatgcttcatgtt

SEQ ID NO: 41 - DNA, 50 bp, Artificial sequence
  Synthesized
aaatacaaatgtgctgaggaagtcccttagaaagaggctgaggctggggt

SEQ ID NO: 42 - DNA, 50 bp, Artificial sequence
  Synthesized
gggagggcaggaaccatgagcagagccagtaaacaaagagtcggatataa

SEQ ID NO: 43 - DNA, 50 bp, Artificial sequence
  Synthesized
ggtggatgatcttctggtagtgtaagtactcgatgcctttgatcagatcc

SEQ ID NO: 44 - DNA, 50 bp, Artificial sequence
  Synthesized
cttgatgtgcccatcttctccgaccaggaggttggaaggtttgatgtcac

SEQ ID NO: 45 - DNA, 35 bp, Artificial sequence
  Synthesized
accttgtagtactgggccaggccgatcatggacag

SEQ ID NO: 46 - DNA, 50 bp, Artificial sequence
  Synthesized
aggtgatgtcggtgatgaaatcaatgttctcgtagccattgtccaccaac

SEQ ID NO: 47 - DNA, 41 bp, Artificial sequence
  Synthesized
tccaccgcttcttctgagagagggtcaaatcccaatgtctg

SEQ ID NO: 48 - DNA, 40 bp, Artificial sequence
  Synthesized
tgacgctcccaacagtagccgaagaacgccttccagctgc

SEQ ID NO: 49 - DNA, 50 bp, Artificial sequence
  Synthesized
cctctacatcaaaaccgttcaggttcaaagcccaggctttctgatgttcc

SEQ ID NO: 50 - DNA, 50 bp, Artificial sequence
  Synthesized
accctctggcgcattttgtggttttccactgagccgaaggatcttggctt

SEQ ID NO: 51 - DNA, 50 bp, Artificial sequence
  Synthesized
tcatcttctttatgtaccaccgagtttaagctgcagagagctgtactgat

SEQ ID NO: 52 - DNA, 50 bp, Artificial sequence
  Synthesized
ggcctcagcagtcttaaccaaattatacagtgtccatcattttgggttca

SEQ ID NO: 53 - DNA, 50 bp, Artificial sequence
  Synthesized
agacaagatctcccaagtcctccatagcagtgtattaaggtttttcggta

SEQ ID NO: 54 - DNA, 49 bp, Artificial sequence
  Synthesized
ctctggtgatattgtgtcagacaggtatagtaggagacaagcagctaca

SEQ ID NO: 55 - DNA, 44 bp, Artificial sequence
  Synthesized
gcatctcgcacctcccgttccaaaaaacgtcacggagctctgag

SEQ ID NO: 56 - DNA, 35 bp, Artificial sequence
  Synthesized
ctgcctggctacagccccggatgtgttaaatgtct

SEQ ID NO: 57 - DNA, 50 bp, Artificial sequence
  Synthesized
tgaggtatacaaacttggctggacttctgatcttgcttgatgtttggatg

SEQ ID NO: 58 - DNA, 50 bp, Artificial sequence
  Synthesized
aattaggcctctggcttatggcttttgacttttgcagtacacatgatgtc

SEQ ID NO: 59 - DNA, 50 bp, Artificial sequence
  Synthesized
ccagtctcttctctcaagaggtgtgacgcagaaaattctagatgcttaag

SEQ ID NO: 60 - DNA, 49 bp, Artificial sequence
  Synthesized
tgctttaagaagaccgtctagcttgtagtggactgagtcagacctggag

SEQ ID NO: 61 - DNA, 50 bp, Artificial sequence
  Synthesized
gcctgtggtccagggaaaggtatgaagatcatataaaccggcggtggaca

SEQ ID NO: 62 - DNA, 50 bp, Artificial sequence
  Synthesized
agcgtagggtactgcagcccagctatggttcagactaaaagccgagaaac

SEQ ID NO: 63 - DNA, 50 bp, Artificial sequence
  Synthesized
cctgtgggcatttggtatcgttcagcgtaaaacactccatgaaccaagtt

SEQ ID NO: 64 - DNA, 50 bp, Artificial sequence
  Synthesized
tgtagggaatgaagaactgtgtcttggtgttgggtagtgggccttttatg

SEQ ID NO: 65 - DNA, 50 bp, Artificial sequence
  Synthesized
cgatagcgcccaccatgcctttatatccatgagggcccgtctctcccttg

SEQ ID NO: 66 - DNA, 35 bp, Artificial sequence
  Synthesized
cctaggaccttcctcacccggtggcccagtggcac

SEQ ID NO: 67 - DNA, 50 bp, Artificial sequence
  Synthesized
cttgcccgtgaaagaaagctgcgtgcacatcaacttcattacccatacca

SEQ ID NO: 68 - DNA, 50 bp, Artificial sequence
  Synthesized
agcaggaaagaggttgattgtgtcaatacggtagttcttgttagtcagtg

SEQ ID NO: 69 - DNA, 50 bp, Artificial sequence
  Synthesized
ctggaggtatccaagagtctgccgagggacttcaagtattcaggaagggg

SEQ ID NO: 70 - DNA, 50 bp, Artificial sequence
  Synthesized
gaagagcccaaacctgcctggcttcaaaacaggtggtgagctccccattg

SEQ ID NO: 71 - DNA, 50 bp, Artificial sequence
  Synthesized
cagcctcaggcgaagtccatttctcaataaataaaaccccctccctccaa

SEQ ID NO: 72 - DNA, 49 bp, Artificial sequence
  Synthesized
cgtttgaacagtgcgttccttgcgcccagcagaaccctgaattggcaaa

SEQ ID NO: 73 - DNA, 50 bp, Artificial sequence
  Synthesized
ggcggaacgcatcatgtacaagctcagtttctatgattatgtccatcagc

SEQ ID NO: 74 - DNA, 50 bp, Artificial sequence
  Synthesized
ctgaaggagtgtaatgggaaactgcttatgagcctcggtggtcatccaga

SEQ ID NO: 75 - DNA, 50 bp, Artificial sequence
  Synthesized
aaatccactccaacatcgaccagggctttggagatctgagctgactgctg

SEQ ID NO: 76 - DNA, 45 bp, Artificial sequence
  Synthesized
ctgctagctattccatggtcttcatcagtataccacattgcctgg

SEQ ID NO: 77 - DNA, 50 bp, Artificial sequence
  Synthesized
gaaaacttcagtaacaagtccttgagcacatgcctctcccgctgttaact

SEQ ID NO: 78 - DNA, 49 bp, Artificial sequence
  Synthesized
caaatgccttcagcctggtccagacttctttctgaaaagtgctatcagg

SEQ ID NO: 79 - DNA, 50 bp, Artificial sequence
  Synthesized
gctcttgtccgggaagggtcacttgataggcaggctgtaatttttccaaa

SEQ ID NO: 80 - DNA, 50 bp, Artificial sequence
  Synthesized
ttgtgctagggtgatgtcaattggacagattctccctttcttcacaatgg

SEQ ID NO: 81 - DNA, 50 bp, Artificial sequence
  Synthesized
aaggtagccacatgtttcagaactgtggactcaaacacgcctggtgtgtg

SEQ ID NO: 82 - DNA, 50 bp, Artificial sequence
  Synthesized
ctttcttccttcttctagatcctggaggattctgagttcttttgaaagac

SEQ ID NO: 83 - DNA, 35 bp, Artificial sequence
  Synthesized
ctgccgcgctccaggcggcgctccccgcccctcgc

SEQ ID NO: 84 - DNA, 50 bp, Artificial sequence
  Synthesized
taggcacactcaaacaacgactggtcctcactcacaactgataaggcttc

SEQ ID NO: 85 - DNA, 50 bp, Artificial sequence
  Synthesized
ccatcttttttctctgtgagtcatttgtcttgcttttggtcaacacggct

SEQ ID NO: 86 - DNA, 50 bp, Artificial sequence
  Synthesized
ccatctaccagctgttcagaacctgacggctttagttgcccttggttctg

SEQ ID NO: 87 - DNA, 50 bp, Artificial sequence
  Synthesized
tgagccattcacctggctagggttacattccattttgatggtgaccctgg

SEQ ID NO: 88 - DNA, 49 bp, Artificial sequence
  Synthesized
ccaccatcttcccgcctttggccacactgcattcatcaggagagttcct

SEQ ID NO: 89 - DNA, 50 bp, Artificial sequence
  Synthesized
acatcatctgaagtcaaatgtggaagaggagtctctctgaggtagtggag

SEQ ID NO: 90 - DNA, 50 bp, Artificial sequence
  Synthesized
ctgtgtttctagcatgcattaaccgtggagagttttgtaaggctttatca

SEQ ID NO: 91 - DNA, 43 bp, Artificial sequence
  Synthesized
catcctgtttccttggctccaccagctcccggaatgctactac

SEQ ID NO: 92 - DNA, 35 bp, Artificial sequence
  Synthesized
ccagcccacagtccaggcccgctggagactatcag

SEQ ID NO: 93 - DNA, 50 bp, Artificial sequence
  Synthesized
tgaaaggtggtacaatatccgaaacattttcatatcctggaggaggtggt

SEQ ID NO: 94 - DNA, 50 bp, Artificial sequence
  Synthesized
gttaacatacactagatcgccctctggcattccttgaggagagaaagcac

SEQ ID NO: 95 - DNA, 50 bp, Artificial sequence
  Synthesized
gggactgtcttatccacaaacaggaagatcgccttttcagaaggaagctg

SEQ ID NO: 96 - DNA, 50 bp, Artificial sequence
  Synthesized
cttcatctttttccttctcgtaaagctgtcccatagttaggctggactgt

SEQ ID NO: 97 - DNA, 39 bp, Artificial sequence
  Synthesized
aagtggtcgttgagggcaatgccagccccagcgtcaaag

SEQ ID NO: 98 - DNA, 50 bp, Artificial sequence
  Synthesized
ccctgttgctgtagccaaattcgttgtcataccaggaaatgagcttgaca

SEQ ID NO: 99 - DNA, 50 bp, Artificial sequence
  Synthesized
tttcaaacaataatcagggatttcctttgtgaagggcagtcttctatgct

SEQ ID NO: 100 - DNA, 50 bp, Artificial sequence
  Synthesized
gtatttggtgggataagaaaaaagtctccttcgcagcaacgtcctcagca

SEQ ID NO: 101 - DNA, 36 bp, Artificial sequence
  Synthesized
cctggttagcaggtcctcgtagcgtttccgcaactc

SEQ ID NO: 102 - DNA, 35 bp, Artificial sequence
  Synthesized
gtgttcgaatcttcccagctctggttggcccgcag

SEQ ID NO: 103 - DNA, 50 bp, Artificial sequence
  Synthesized
tgaagatgaagatgaccgagagccatactcggccaatggcagtagaatgc

SEQ ID NO: 104 - DNA, 50 bp, Artificial sequence
  Synthesized
tttctcatcaccccacacactctctgcagccaccaccagcaccatgattc

SEQ ID NO: 105 - DNA, 43 bp, Artificial sequence
  Synthesized
ggatgagcctctcacctgtggtgatgttattcaccaaaaccgc

SEQ ID NO: 106 - DNA, 40 bp, Artificial sequence
  Synthesized
taattcctctgcagggtctttaactggtcttgcagcactc

SEQ ID NO: 107 - DNA, 50 bp, Artificial sequence
  Synthesized
cttggctgccccaactggcttcttaggtttggttccgcccgcctttttaa

SEQ ID NO: 108 - DNA, 35 bp, Artificial sequence
  Synthesized
ttcggagttgcgccgccagccgccttcttgggctt

SEQ ID NO: 109 - DNA, 41 bp, Artificial sequence
  Synthesized
gcgctccttggaggcggcaacagctttagtaatgagctcgg

SEQ ID NO: 110 - DNA, 40 bp, Artificial sequence
  Synthesized
ctgccagcgctttcttgagagcggccaaagatacgccgct

SEQ ID NO: 111 - DNA, 36 bp, Artificial sequence
  Synthesized
cttggtgacggccttggtgccctctgacacggcgtg

SEQ ID NO: 112 - DNA, 50 bp, Artificial sequence
  Synthesized
agcctttgggattgggtatgaagacgttagaattacttagagctggtgta

SEQ ID NO: 113 - DNA, 45 bp, Artificial sequence
  Synthesized
tatacttggtgacagccttggtaccttcggacactgcgtgcttgg

SEQ ID NO: 114 - DNA, 50 bp, Artificial sequence
  Synthesized
aagagcctttgagttttaaagcacctaagcacacatttacttggagcttg

SEQ ID NO: 115 - DNA, 35 bp, Artificial sequence
  Synthesized
cggagcaaccggtgcacgcggcccacggggaactg

SEQ ID NO: 116 - DNA, 35 bp, Artificial sequence
  Synthesized
cgccggcgcccacgcgctccgaatagttgcccttg

SEQ ID NO: 117 - DNA, 35 bp, Artificial sequence
  Synthesized
gctgggcagggacatggatggtagcctgcatggtc

SEQ ID NO: 118 - DNA, 44 bp, Artificial sequence
  Synthesized
agtgatgcctaccaactgtgggtcatcctcagggccatcttcat

SEQ ID NO: 119 - DNA, 48 bp, Artificial sequence
  Synthesized
tgaatttttttcatccatgggtagactatgggttgcttgctggcggcg

SEQ ID NO: 120 - DNA, 45 bp, Artificial sequence
  Synthesized
cgcttgggttcccctccgttataattggggttcaccgtgctaacg

SEQ ID NO: 121 - DNA, 50 bp, Artificial sequence
  Synthesized
ggtcgagaaatgcctcactggatcataggcggtggaattgagggcgacgt

SEQ ID NO: 122 - DNA, 50 bp, Artificial sequence
  Synthesized
gaataaaagggagtcgagtagatccggttctgggcaacggccgctccata

SEQ ID NO: 123 - DNA, 40 bp, Artificial sequence
  Synthesized
ccgagagatgctgtcctcacacacaaagggaccaccgctg

SEQ ID NO: 124 - DNA, 36 bp, Artificial sequence
  Synthesized
ccaactcacaatgccacacagccgccaacgtggcgt

SEQ ID NO: 125 - DNA, 50 bp, Artificial sequence
  Synthesized
tgagcacacagagggctacaatgtgatggcctcccatctccttcatcaca

SEQ ID NO: 126 - DNA, 50 bp, Artificial sequence
  Synthesized
cagtgctttgatgtaatccagcaggtcagcaaagaatttatagcccccct

SEQ ID NO: 127 - DNA, 50 bp, Artificial sequence
  Synthesized
aatcgtgactttcagttgcggtagtacacgttccacttctaggctccatt

SEQ ID NO: 128 - DNA, 50 bp, Artificial sequence
  Synthesized
tgctggtgcatttggtcaacatggattctccaatccttattgtcagtcct

SEQ ID NO: 129 - DNA, 50 bp, Artificial sequence
  Synthesized
cgggcgcatgaagtctgggtgctgtgctcgagtctctgaatattttgata

SEQ ID NO: 130 - DNA, 38 bp, Artificial sequence
  Synthesized
tggtcggccgcttcgaccaacatgtggtgagcattcca

SEQ ID NO: 131 - DNA, 50 bp, Artificial sequence
  Synthesized
tctttgagaaaatcaatgtccctggaggagatgatgcccaccaagcggct

SEQ ID NO: 132 - DNA, 45 bp, Artificial sequence
  Synthesized
tccctctttgtcattatctcttccaagaaacagtcatgttcctcc

SEQ ID NO: 133 - DNA, 50 bp, Artificial sequence
  Synthesized
atctggcatttttaagatggcaaagcacttttgcatcctgtgggctgttg

SEQ ID NO: 134 - DNA, 45 bp, Artificial sequence
  Synthesized
tgctagagacctggtgttgatatccacattcataggctctgagtg

SEQ ID NO: 135 - DNA, 50 bp, Artificial sequence
  Synthesized
agaccacaccatcgaggtcttcacagcggcgatcatcacactcacaagtc

SEQ ID NO: 136 - DNA, 50 bp, Artificial sequence
  Synthesized
tcctctctcacaaacacagcgaccacaggaacatgtgccgtggcctccac

SEQ ID NO: 137 - DNA, 45 bp, Artificial sequence
  Synthesized
gacaatctctatctgcgccgtgtgcttggcataaaactccagggc

SEQ ID NO: 138 - DNA, 50 bp, Artificial sequence
  Synthesized
catatgctgggcacgggaaagactatctgttccattgttcggtctaatct

SEQ ID NO: 139 - DNA, 50 bp, Artificial sequence
  Synthesized
cttggacactaaggatcaggtgagcttcctcagttggaattactttgtac

SEQ ID NO: 140 - DNA, 50 bp, Artificial sequence
  Synthesized
gtcaattattcaagtactccatactcgtcctacagacccccagtaaaaac

SEQ ID NO: 141 - DNA, 46 bp, Artificial sequence
  Synthesized
tgaggaagacaaccgggacccacatggtgacacagctctccgggtg

SEQ ID NO: 142 - DNA, 50 bp, Artificial sequence
  Synthesized
aatccgagacaggatgaggggtgcagcaccaatccacgtcacggacaggg

SEQ ID NO: 143 - DNA, 36 bp, Artificial sequence
  Synthesized
atcacgcttttgttcctgatgcagtgggcagctgtg

SEQ ID NO: 144 - DNA, 42 bp, Artificial sequence
  Synthesized
cctgtgtcttcaggatgaaacaggctgtgccgacccagcaag

SEQ ID NO: 145 - DNA, 36 bp, Artificial sequence
  Synthesized
cccagccagaaacgaggcaagagttccccgcggtag

SEQ ID NO: 146 - DNA, 35 bp, Artificial sequence
  Synthesized
cagcacggtaggcattctgccgttcgccagcagac

SEQ ID NO: 147 - DNA, 50 bp, Artificial sequence
  Synthesized
gagagtatggatgaaccactctctgcagccaaaacagaacgaagcgggga

SEQ ID NO: 148 - DNA, 50 bp, Artificial sequence
  Synthesized
acaggaattgaaaaggcaagacccccgtccacaaggggaggcgagggaat

SEQ ID NO: 149 - DNA, 50 bp, Artificial sequence
  Synthesized
atttattttaccccctagctgattttctattacagcatatcagtctaggg

SEQ ID NO: 150 - DNA, 50 bp, Artificial sequence
  Synthesized
tcttaccattattattaatcttacttgctttcagcatgcagagagctctt

SEQ ID NO: 151 - DNA, 50 bp, Artificial sequence
  Synthesized
tatctccagacttgaagatagtctgaccccaacgcctcctaccactttta

SEQ ID NO: 152 - DNA, 50 bp, Artificial sequence
  Synthesized
cttcttggaatgggaggctccgaaatcatagtcctccaactcttcccagc

SEQ ID NO: 153 - DNA, 49 bp, Artificial sequence
  Synthesized
ctctcgctcctcgccgttgaccagacaggagaaaaggccaaaggactcg

SEQ ID NO: 154 - DNA, 36 bp, Artificial sequence
  Synthesized
ccgcgggatgaacctgaacacagcccggtgagtctg

SEQ ID NO: 155 - DNA, 50 bp, Artificial sequence
  Synthesized
tgtgctgaaactagactgtcaactctgtaagagcttggaccaagtctgtc

SEQ ID NO: 156 - DNA, 50 bp, Artificial sequence
  Synthesized
aaacaaagagctcaaggcctcaccttggtttattcactgctggttttcta

SEQ ID NO: 157 - DNA, 45 bp, Artificial sequence
  Synthesized
tgtgttctctccttctaccagcaccgtgatactacgagggatatt

SEQ ID NO: 158 - DNA, 48 bp, Artificial sequence
  Synthesized
caagaaaataccagtgacgctgacgtggtctccaggctgggcaatcct

SEQ ID NO: 159 - DNA, 50 bp, Artificial sequence
  Synthesized
aactccaattgtgtcagatccagaaaggctgagcccataaagtcatcctg

SEQ ID NO: 160 - DNA, 47 bp, Artificial sequence
  Synthesized
gataatgaggatctttcagagtaagggtcacatctgtgggcctgttt

SEQ ID NO: 161 - DNA, 38 bp, Artificial sequence
  Synthesized
cgagcagacagaaggcactggtgggtcacatctcgggc

SEQ ID NO: 162 - DNA, 50 bp, Artificial sequence
  Synthesized
ggggctggggagtgagagggacaaggcagggcatgattgattaaagctaa

SEQ ID NO: 163 - DNA, 50 bp, Artificial sequence
  Synthesized
tcttgctttttctattgacttgagtttctccttcgcttggtaaacagctg

SEQ ID NO: 164 - DNA, 50 bp, Artificial sequence
  Synthesized
ctgatcctatgtgcatacttaattatttcttcagaggagatagcaccttt

SEQ ID NO: 165 - DNA, 35 bp, Artificial sequence
  Synthesized
gaatgtgcaggtggcatccctgaggattcagagct

SEQ ID NO: 166 - DNA, 42 bp, Artificial sequence
  Synthesized
tatcgtggtaaaggctaggctgggaccccggacagagtatga

SEQ ID NO: 167 - DNA, 50 bp, Artificial sequence
  Synthesized
aaatttattattcctccgaaatccaaagtcccagccacatatggtcagcc

SEQ ID NO: 168 - DNA, 50 bp, Artificial sequence
  Synthesized
gtcaaggtgcagctctcatttccaaggagaactctagttttctttaaatc

SEQ ID NO: 169 - DNA, 50 bp, Artificial sequence
  Synthesized
gatctatgcaacttctgataggactccaactcccttacactgctggaaac

SEQ ID NO: 170 - DNA, 50 bp, Artificial sequence
  Synthesized
cctttcagccacagaaacgattgacatgcttctctccccaacccctagaa

SEQ ID NO: 171 - DNA, 50 bp, Artificial sequence
  Synthesized
aagaggcaatagaaaagcaggtaccaataggtctggccgtgtgggaagtc

SEQ ID NO: 172 - DNA, 50 bp, Artificial sequence
  Synthesized
acatggtgagagccgagtagggaacatggaaacacgtgaccattgtttca

SEQ ID NO: 173 - DNA, 50 bp, Artificial sequence
  Synthesized
ggttggaacaagcaggagagagaaacaattcaaccagggtctgggtggtc

SEQ ID NO: 174 - DNA, 45 bp, Artificial sequence
  Synthesized
caggtcatgccaggatgggttttgggagaagcccagagtgaaaag

SEQ ID NO: 175 - DNA, 50 bp, Artificial sequence
  Synthesized
cggttgagatttcaccaaggttctggttctggaatgagtcactggctaag

SEQ ID NO: 176 - DNA, 50 bp, Artificial sequence
  Synthesized
ttctggattttctccatcagtctaggactgaagacaccgatctctggtgt

SEQ ID NO: 177 - DNA, 50 bp, Artificial sequence
  Synthesized
aaagcagcgaccatccagtcatttatttccctccattcccaatgatgtac

SEQ ID NO: 178 - DNA, 50 bp, Artificial sequence
  Synthesized
caggcacgggctcaggcaccgcttgtctggaatgtcaatttgaaacttaa

SEQ ID NO: 179 - DNA, 48 bp, Artificial sequence
  Synthesized
ctgatggcattagattcctgcacgctaagagttctccctctacatctg

SEQ ID NO: 180 - DNA, 50 bp, Artificial sequence
  Synthesized
gtctttctcttcacctactgatggtttaggcgtgtgcatggctttgcctg

SEQ ID NO: 181 - DNA, 50 bp, Artificial sequence
  Synthesized
tagggctggaacaaggactcttttctctggacagcttgcacctacaatcc

SEQ ID NO: 182 - DNA, 50 bp, Artificial sequence
  Synthesized
ccaaaggaatattgcaaatacccaaggtcaccctgtcaggagtggcagaa

SEQ ID NO: 183 - DNA, 45 bp, Artificial sequence
  Synthesized
tcagtgggtagcgaaaggtgtagaaggcggacatcagggccttgg

SEQ ID NO: 184 - DNA, 41 bp, Artificial sequence
  Synthesized
atataggtgttgaacgcccctgcagtcatctgggctgagac

SEQ ID NO: 185 - DNA, 50 bp, Artificial sequence
  Synthesized
catttagatcctaaaactgtggggagtggggacagggtgaacgaggtgcc

SEQ ID NO: 186 - DNA, 50 bp, Artificial sequence
  Synthesized
cccagtgattctgatgtgggatagtctagaagaatagttccagaggcaat

SEQ ID NO: 187 - DNA, 50 bp, Artificial sequence
  Synthesized
caggatttccagaatttggtaaaaaggcatggcctaagataccacctggc

SEQ ID NO: 188 - DNA, 50 bp, Artificial sequence
  Synthesized
tccagtgtctgaagctgaccagtgttcattcttgtcaaaatggacaactc

SEQ ID NO: 189 - DNA, 50 bp, Artificial sequence
  Synthesized
tttcttgaagagcaggtgaggcgcccttgcttaaaagggaagcgcccagg

SEQ ID NO: 190 - DNA, 50 bp, Artificial sequence
  Synthesized
ttaaaagaaccagagttcaagtttcagccccctgggtctccctctcgctg

SEQ ID NO: 191 - DNA, 50 bp, Artificial sequence
  Synthesized
tttttgggtccttcttctccaccacgatatacttgcagtcctccttcttg

SEQ ID NO: 192 - DNA, 50 bp, Artificial sequence
  Synthesized
gtgcctactagaagcacattagattatccattcactgacagaacaggtct

SEQ ID NO: 193 - DNA, 50 bp, Artificial sequence
  Synthesized
gaagtgaatgaaagtttgacactggcactggagtaaccctcgtcactccc

SEQ ID NO: 194 - DNA, 50 bp, Artificial sequence
  Synthesized
tggcccagtgaatattttgccctgcactgttatgtcatgctgggttctat

SEQ ID NO: 195 - DNA, 50 bp, Artificial sequence
  Synthesized
atgatcgtgtgacgcaagtcaagttctaggaaacccaagtagtcatccag

SEQ ID NO: 196 - DNA, 50 bp, Artificial sequence
  Synthesized
tgaggtccggaatcatgtccaatctgcatttctctggtgattttgcagga

SEQ ID NO: 197 - DNA, 50 bp, Artificial sequence
  Synthesized
attctcagcaccgtctagctggaattggtcaaaaccagactcctctagtt

SEQ ID NO: 198 - DNA, 50 bp, Artificial sequence
  Synthesized
tgaatggaatcaagattgaggtctatagtctctgaatgccctaggttctg

SEQ ID NO: 199 - DNA, 50 bp, Artificial sequence
  Synthesized
tttctcacacacagatttaggaatgaccaacttgtaccctcccagcgttt

SEQ ID NO: 200 - DNA, 50 bp, Artificial sequence
  Synthesized
ttctcctagtaatctgcaatgcaatcacaatgcccaaactagacctgcca

SEQ ID NO: 201 - DNA, 50 bp, Artificial sequence
  Synthesized
cactgtgttcaaggcccacttccaccaaaaatctagctgtgtggcctcaa

SEQ ID NO: 202 - DNA, 50 bp, Artificial sequence
  Synthesized
gaactcagagagcagacactgggttttacagtcagaaactgcagaaagta

SEQ ID NO: 203 - DNA, 50 bp, Artificial sequence
  Synthesized
ctggcatatcacagtgggattcgaaacacgtgcattatctgaaccccact

SEQ ID NO: 204 - DNA, 50 bp, Artificial sequence
  Synthesized
ctcgaaaggtactccagtaaacccatccactcctcttcaatgctgtcttc

SEQ ID NO: 205 - DNA, 50 bp, Artificial sequence
  Synthesized
ctttgagagcattggctaggttgcagtaagcatcagggaaatgtggttgt

SEQ ID NO: 206 - DNA, 50 bp, Artificial sequence
  Synthesized
acggagagctgtattataacaatcttctgcttcagcaacactgcccttct

SEQ ID NO: 207 - DNA, 50 bp, Artificial sequence
  Synthesized
gagcgtgcaggctgcgttccgtccttacgatgaagaccacgatgcagttt

SEQ ID NO: 208 - DNA, 50 bp, Artificial sequence
  Synthesized
ggataaggccaggtcaatggctgcaagcatgcagagaaagaggtacatcg

SEQ ID NO: 209 - DNA, 41 bp, Artificial sequence
  Synthesized
agctgggactggagtgtgaacaaactgtcttccaggttccg

SEQ ID NO: 210 - DNA, 35 bp, Artificial sequence
  Synthesized
gctgggcacctgtggaagcactttgcaacagttgc

SEQ ID NO: 211 - DNA, 50 bp, Artificial sequence
  Synthesized
taaggaacacatcaattcattttctaatgtccttccctcacaagcgggac

SEQ ID NO: 212 - DNA, 50 bp, Artificial sequence
  Synthesized
tcccgttcaaataaatatccacaacaggatctgttttcctgcccatcctt

SEQ ID NO: 213 - DNA, 50 bp, Artificial sequence
  Synthesized
acatcgccgtccagcatgcggatgcctcctattttggcattgtacgctat

SEQ ID NO: 214 - DNA, 50 bp, Artificial sequence
  Synthesized
cgatgtagttgggtctgatgcccagcgactttgcctcgaccacatctgtg

SEQ ID NO: 215 - DNA, 50 bp, Artificial sequence
  Synthesized
ctcaaagtccaatgacagaaaatgaaatatgctcgggtccggcgcggcgc

SEQ ID NO: 216 - DNA, 42 bp, Artificial sequence
  Synthesized
ggccaaccagtgacacactgtagttgctcatggttctaatgg

SEQ ID NO: 217 - DNA, 50 bp, Artificial sequence
  Synthesized
gtgattgctcggatagtgattcccagttgttggtgtttcatgcagagttg

SEQ ID NO: 218 - DNA, 50 bp, Artificial sequence
  Synthesized
ttagaaaacaggccagcttcacctgggcaccctgctgcctttcaaggctg

SEQ ID NO: 219 - DNA, 50 bp, Artificial sequence
  Synthesized
cactttcatccagtcgcctttcagttcccagggccaagaggttattgtat

SEQ ID NO: 220 - DNA, 50 bp, Artificial sequence
  Synthesized
aggaggaaactgccttctccaggttgatccacgtctgaagttcttgtcat

SEQ ID NO: 221 - DNA, 50 bp, Artificial sequence
  Synthesized
tgctctgtgatactactcttgctttcagagttggaatgattgacaaaggc

SEQ ID NO: 222 - DNA, 45 bp, Artificial sequence
  Synthesized
ctagcagaagaggcagagaaggtattttgagctggtgctggtatc

SEQ ID NO: 223 - DNA, 35 bp, Artificial sequence
  Synthesized
tcaaaggaggccctcagggagttgatctccgtctg

SEQ ID NO: 224 - DNA, 35 bp, Artificial sequence
  Synthesized
agctgcccacattctccatttgctgcttcaaggag

SEQ ID NO: 225 - DNA, 50 bp, Artificial sequence
  Synthesized
tttcttccctgcttcagcagtatccacagctgccagtatgaaaatgaatg

SEQ ID NO: 226 - DNA, 45 bp, Artificial sequence
  Synthesized
ccattctccacagtcagacttcttcacttttttttctggtttctc

SEQ ID NO: 227 - DNA, 50 bp, Artificial sequence
  Synthesized
caagagtttaagccacaaatacatggtcatatctggaagtcagccgtgtc

SEQ ID NO: 228 - DNA, 50 bp, Artificial sequence
  Synthesized
ctttgccctgtcacaaatacttctgtgtccagaaaggcaaagccaaatgc

SEQ ID NO: 229 - DNA, 50 bp, Artificial sequence
  Synthesized
aaaatacttgaacgaagctccatgcagctgacaggcacagccatcttgag

SEQ ID NO: 230 - DNA, 50 bp, Artificial sequence
  Synthesized
agcgttattccccagaccactgaagatcactgtaaatccatcaggctcag

SEQ ID NO: 231 - DNA, 50 bp, Artificial sequence
  Synthesized
acagcactttcctgggagccatgtgacgccagatcttcctctggcagttc

SEQ ID NO: 232 - DNA, 50 bp, Artificial sequence
  Synthesized
ggaacaggcacaggcatcggggaatcagatggtatcagtggggatagggc

SEQ ID NO: 233 - DNA, 50 bp, Artificial sequence
  Synthesized
ctggaaaaactgcgagacattcctgcagtcccggaacaagaactccaggc

SEQ ID NO: 234 - DNA, 50 bp, Artificial sequence
  Synthesized
acagcattgaagagttctcgttcactaagggcttccttgactcctccttt

SEQ ID NO: 235 - DNA, 50 bp, Artificial sequence
  Synthesized
actagagggtaaacttctcggtctaaatcaaagccaagctcctcttcggc

SEQ ID NO: 236 - DNA, 50 bp, Artificial sequence
  Synthesized
catggcaatggccaaaatactcgtctccttcatccaccacggcatgtacc

SEQ ID NO: 237 - DNA, 50 bp, Artificial sequence
  Synthesized
ttgccagtcgctggttttcatccagagcacgaagctcgtggtctgaatac

SEQ ID NO: 238 - DNA, 50 bp, Artificial sequence
  Synthesized
cagcaatatatcctgttcatcttcttcatcatgaaggtcagctttcttct

SEQ ID NO: 239 - DNA, 50 bp, Artificial sequence
  Synthesized
gagatacaaagtaccagaagcgggacttggcgacgacatgattaggcgca

SEQ ID NO: 240 - DNA, 50 bp, Artificial sequence
  Synthesized
ctgcccacagtagacaatctcccctgaagacttcttcatcttctttaact

SEQ ID NO: 241 - DNA, 50 bp, Artificial sequence
  Synthesized
aaatccgaaaggatctcatcccattaggacccttgtctccttttctgttg

SEQ ID NO: 242 - DNA, 49 bp, Artificial sequence
  Synthesized
catttatggctgtcaacccgccagttctcaggagtttgtataaaagcct

SEQ ID NO: 243 - DNA, 40 bp, Artificial sequence
  Synthesized
ctgataaccttgttgagccggtcgtcgtccgcctcgatac

SEQ ID NO: 244 - DNA, 50 bp, Artificial sequence
  Synthesized
tgccaataccctgggcaatgacgtcttcaatgttttttccattcagctca

SEQ ID NO: 245 - DNA, 50 bp, Artificial sequence
  Synthesized
gaaatgtctccaggcaaactgttccttcacgtagcctcgggacttgagag

SEQ ID NO: 246 - DNA, 50 bp, Artificial sequence
  Synthesized
tgaaggtaatcacggagatactggataccctcattggtaaggtaccagta

SEQ ID NO: 247 - DNA, 50 bp, Artificial sequence
  Synthesized
cagcaggaccctcttcttgttttgaaagatggtcggctgcttttggtagg

SEQ ID NO: 248 - DNA, 50 bp, Artificial sequence
  Synthesized
agaccgatgttcttgtagtaccgcgggagcttctccttgccagtttctcc

SEQ ID NO: 249 - DNA, 50 bp, Artificial sequence
  Synthesized
agaaagttctcttagaagatgaccattccatacaaaccgctgatctgccc

SEQ ID NO: 250 - DNA, 50 bp, Artificial sequence
  Synthesized
ataaagccatgtaacactggaagggcaaaccgatgaacctctggctgtgc

SEQ ID NO: 251 - DNA, 50 bp, Artificial sequence
  Synthesized
ccaggtacatggtgaaagtgccttatacaggttgaataaaaatcactgcc

SEQ ID NO: 252 - DNA, 50 bp, Artificial sequence
  Synthesized
accttgtgtccccagcatctagattgctgaaaaagatgtagatgttgctt

SEQ ID NO: 253 - DNA, 50 bp, Artificial sequence
  Synthesized
ctctaagcccaaccagaagagtcagctagaagagccaataggtgcacaga

SEQ ID NO: 254 - DNA, 50 bp, Artificial sequence
  Synthesized
gagctgatgacccaagtggactaaacacggagctagcagaaacaggcaga

SEQ ID NO: 255 - DNA, 50 bp, Artificial sequence
  Synthesized
cgggcctggagtcacagttatcctggaaaatgcgtggaaaaggaacaggc

SEQ ID NO: 256 - DNA, 50 bp, Artificial sequence
  Synthesized
gaacagatcaacggcaaaagccgaatttgctagttgcagggcatccattg

SEQ ID NO: 257 - DNA, 50 bp, Artificial sequence
  Synthesized
cagcctctcttcccactgtatggatcttttactaagctgatctctccatt

SEQ ID NO: 258 - DNA, 45 bp, Artificial sequence
  Synthesized
cccggctgttttcttcttgtcctgaactgttctccgctgttcctg

SEQ ID NO: 259 - DNA, 46 bp, Artificial sequence
  Synthesized
ttaatgtaggtcgtgcgcatttgccgggctcggtggcgccgcagcc

SEQ ID NO: 260 - DNA, 50 bp, Artificial sequence
  Synthesized
atccgcaagtcggcggcggggtccaattcaaacagctgtctctgcataaa

SEQ ID NO: 261 - DNA, 35 bp, Artificial sequence
  Synthesized
ctgccacccaccgccatggctgcttcggctcccgg

SEQ ID NO: 262 - DNA, 50 bp, Artificial sequence
  Synthesized
gaagcagaaagagggcaagtttgcccaaagcgtgagggttctgtctccat

SEQ ID NO: 263 - DNA, 50 bp, Artificial sequence
  Synthesized
ggtgtgggtggcaactctgacaaataagccaattctttttgtgttcgtgg

SEQ ID NO: 264 - DNA, 50 bp, Artificial sequence
  Synthesized
ctggtggtgaagttctttctggtgaacttgagtcttctgaaacatgaaga

SEQ ID NO: 265 - DNA, 50 bp, Artificial sequence
  Synthesized
ccatatacaacaaatccgatatggatccctttcttgccacgggaaggctc

SEQ ID NO: 266 - DNA, 50 bp, Artificial sequence
  Synthesized
tctaactagtaagacaggtgggaggttctttgtgaggatttccaaccaag

SEQ ID NO: 267 - DNA, 50 bp, Artificial sequence
  Synthesized
ttgacttcctcaggggcaggaaaggcttcgatgggccagttgagggtgca

SEQ ID NO: 268 - DNA, 46 bp, Artificial sequence
  Synthesized
cttgtggtccagggccagcccactcagcttgatcttcttcgtgtaa

SEQ ID NO: 269 - DNA, 49 bp, Artificial sequence
  Synthesized
catcatcagcatccagacactgaagctccacgttcctgaagatgagcgg

SEQ ID NO: 270 - DNA, 50 bp, Artificial sequence
  Synthesized
cacttcgtcgtattcatcccgaccttcctcctcatcaaaggttgccttgg

SEQ ID NO: 271 - DNA, 50 bp, Artificial sequence
  Synthesized
gagtactttgctgttgaatggttcctgtgccatacagagataagatggag

SEQ ID NO: 272 - DNA, 50 bp, Artificial sequence
  Synthesized
tggtgcttgtgaggtaaatggtatatttgtgggtcccataaatacaccag

SEQ ID NO: 273 - DNA, 44 bp, Artificial sequence
  Synthesized
ttcatggcgatgcccagcttgcccgtgcacagcctctgggagat

SEQ ID NO: 274 - DNA, 50 bp, Artificial sequence
  Synthesized
ggtagcccaggatgaagatgatccagaagagggccacgccgcccagcacc

SEQ ID NO: 275 - DNA, 50 bp, Artificial sequence
  Synthesized
actgggagcaaagatatttcttaggcttcaggttcgtagtcttgataccc

SEQ ID NO: 276 - DNA, 50 bp, Artificial sequence
  Synthesized
ggaactgagcacttgtacaggatggaacatctgtcagcagatctcaagaa

SEQ ID NO: 277 - DNA, 50 bp, Artificial sequence
  Synthesized
cgtataactgctcgtatcactgtgagactacaagcagcaaataaatggga

SEQ ID NO: 278 - DNA, 50 bp, Artificial sequence
  Synthesized
atggttacttcatctcaatttacagtggcccaatgttattttatcccatg

SEQ ID NO: 279 - DNA, 50 bp, Artificial sequence
  Synthesized
aagttctgcgtccagaggtcagttgaaaactgcaccgcacttaccacgtc

SEQ ID NO: 280 - DNA, 50 bp, Artificial sequence
  Synthesized
caacagggccaaggcactgagaagaaagatgcctgttaccttcatggctg

SEQ ID NO: 281 - DNA, 44 bp, Artificial sequence
  Synthesized
catttattcacttctcaagtggcccccgcttggatgcgccctcg

SEQ ID NO: 282 - DNA, 50 bp, Artificial sequence
  Synthesized
aacgcagagagatccataacatggaaacactgacgcttccgaaaccgccc

SEQ ID NO: 283 - DNA, 50 bp, Artificial sequence
  Synthesized
taaagtaactgccaactgggactgtatgtcacctaagtcaggataactcc

SEQ ID NO: 284 - DNA, 50 bp, Artificial sequence
  Synthesized
ccatgttctaaagtttctaagagtcttgaggttatgctagggctcctggt

SEQ ID NO: 285 - DNA, 46 bp, Artificial sequence
  Synthesized
ccacaaggcagggagagaagggagccacataagtagattcctggcg

SEQ ID NO: 286 - DNA, 35 bp, Artificial sequence
  Synthesized
atggtaggcatcatgaagggcacagtgctcgctgc

SEQ ID NO: 287 - DNA, 50 bp, Artificial sequence
  Synthesized
tccgaccccgcaatcttataaaaactcctcattcggcttgttctcagctc

SEQ ID NO: 288 - DNA, 50 bp, Artificial sequence
  Synthesized
ggtctttgaaaacgcgcagtaggagggtgattcctattacgcgcccacac

SEQ ID NO: 289 - DNA, 50 bp, Artificial sequence
  Synthesized
tttttcctcaaaatcccaccgaggctcagatttgaagttggcggccttca

SEQ ID NO: 290 - DNA, 50 bp, Artificial sequence
  Synthesized
ttcacagagtcagggcaagtcgtctgaaggcctcctatttcgaagctgta

SEQ ID NO: 291 - DNA, 50 bp, Artificial sequence
  Synthesized
atatataaacctgccggctggcatccttaggtcctaactgaagtgcccaa

SEQ ID NO: 292 - DNA, 50 bp, Artificial sequence
  Synthesized
ctggcgggcaagttcaataacctgttgtcgcgcttgaatattgttgctgc

SEQ ID NO: 293 - DNA, 50 bp, Artificial sequence
  Synthesized
atcaaagataagttgaaggagcgtgtgttctgtgtacctttgcaaccagt

SEQ ID NO: 294 - DNA, 50 bp, Artificial sequence
  Synthesized
ccatgactctactcaatgtcgtccaactttttgtatccttgcttgggttt

SEQ ID NO: 295 - DNA, 35 bp, Artificial sequence
  Synthesized
gagtcggggagccgctgggcttcggagtcccgtgt

SEQ ID NO: 296 - DNA, 50 bp, Artificial sequence
  Synthesized
ccaaaatccatccgcaaggtccaaggcccttactggggctgtccttgaag

SEQ ID NO: 297 - DNA, 37 bp, Artificial sequence
  Synthesized
atgaggtctgtgaggtaatccttggagtagtcggagc

SEQ ID NO: 298 - DNA, 46 bp, Artificial sequence
  Synthesized
gtacatcttcttggacgtgcggaagaagctcacgctgtcattggtg

SEQ ID NO: 299 - DNA, 50 bp, Artificial sequence
  Synthesized
ccctcaattcatattttattcttgagccgcttggtcaggtttgattcgca

SEQ ID NO: 300 - DNA, 47 bp, Artificial sequence
  Synthesized
tcagcctccaaattgctgggattacagacatgacctaccgtcccggg

SEQ ID NO: 301 - DNA, 50 bp, Artificial sequence
  Synthesized
aatgtgacatcgctttctccataaccttcctcctccttaaccaaccccca

SEQ ID NO: 302 - DNA, 47 bp, Artificial sequence
  Synthesized
tcgtgttctcctgaggctgcttggtccttcgatgctgattaactgag

SEQ ID NO: 303 - DNA, 50 bp, Artificial sequence
  Synthesized
gcacgaagtgcaatggtctttaggtcaagtttacaaccaagattcactgt

SEQ ID NO: 304 - DNA, 50 bp, Artificial sequence
  Synthesized
tcctcatgattaccgcagcaaaccgcttgggattatattcggcgtttcgg

SEQ ID NO: 305 - DNA, 50 bp, Artificial sequence
  Synthesized
tgtttctagactgtatatctgctaactggcaccgtattccctgaaaggga

SEQ ID NO: 306 - DNA, 50 bp, Artificial sequence
  Synthesized
cccagcaacacacatctggaatcttgttatggcttcttcagaccaatgtt

SEQ ID NO: 307 - DNA, 50 bp, Artificial sequence
  Synthesized
gcctgtgtaactgttgatagatccaagttaaacttctccattaactgccg

SEQ ID NO: 308 - DNA, 50 bp, Artificial sequence
  Synthesized
acgctaagaaggcggaagtagcctccagctcaccactattttttaggaag

SEQ ID NO: 309 - DNA, 50 bp, Artificial sequence
  Synthesized
cgcaagaccccaaagagtttgcgacgcatgttcctcccagccttgaagcc

SEQ ID NO: 310 - DNA, 50 bp, Artificial sequence
  Synthesized
tctggaggctgttcacctgcaaatccagaaacaggctgtgacacttcagc

SEQ ID NO: 311 - DNA, 50 bp, Artificial sequence
  Synthesized
ttcctctgcaccttctcgcagaccttcatggagaaatgccgtaggccctt

SEQ ID NO: 312 - DNA, 50 bp, Artificial sequence
  Synthesized
tgaactccgcaaccagctcgtctgccacttcgttgtaggaagtggtccct

SEQ ID NO: 313 - DNA, 50 bp, Artificial sequence
  Synthesized
tctgcagggaaggagaactggcttgatcttcaggactcttgaagggatgt

SEQ ID NO: 314 - DNA, 50 bp, Artificial sequence
  Synthesized
ggcacttcttattagctggcagcaagaggtcaggtggtaatggccaaagc

SEQ ID NO: 315 - DNA, 39 bp, Artificial sequence
  Synthesized
acgttgctgccgtcggccagcagcagagcagtgtcggtg

SEQ ID NO: 316 - DNA, 35 bp, Artificial sequence
  Synthesized
ccccgatgccttcggcctcctcagccaggaggtac

SEQ ID NO: 317 - DNA, 45 bp, Artificial sequence
  Synthesized
gcatacagcaggagtgagtggatgtgctggtccagcggaggccgg

SEQ ID NO: 318 - DNA, 50 bp, Artificial sequence
  Synthesized
ggtcccggaagatcacctctagggagatactaacacaccctccgaacaga

SEQ ID NO: 319 - DNA, 50 bp, Artificial sequence
  Synthesized
agcaaataaccaacagccaatgtagtcattgggtaggataagcaggcggt

SEQ ID NO: 320 - DNA, 45 bp, Artificial sequence
  Synthesized
cccattagatgctgaagggcagttcatttttcaagggctcactca

SEQ ID NO: 321 - DNA, 50 bp, Artificial sequence
  Synthesized
aatgaatcagccaatctaatcccattgctcccagctgttcaactaagccc

SEQ ID NO: 322 - DNA, 50 bp, Artificial sequence
  Synthesized
gctcctggagcagagtgatgtattattctgccagggctttacaactaatg

SEQ ID NO: 323 - DNA, 50 bp, Artificial sequence
  Synthesized
cttccagtagagatcgctgttgccctgtactttgccgaatgtgtaactga

SEQ ID NO: 324 - DNA, 50 bp, Artificial sequence
  Synthesized
gccagcgcgggccgagagtggaattcccggatgaggcggtaacgctgcgc

SEQ ID NO: 325 - DNA, 36 bp, Artificial sequence
  Synthesized
ctcggcggctgctgccggtctggctcttcctcgctg

SEQ ID NO: 326 - DNA, 35 bp, Artificial sequence
  Synthesized
tgctgctgcgccgcttgcgtcccccgcgcttgccg

SEQ ID NO: 327 - DNA, 40 bp, Artificial sequence
  Synthesized
acgaggtttgtcacctggtatgcactgagccgagtgactg

SEQ ID NO: 328 - DNA, 44 bp, Artificial sequence
  Synthesized
tccccttcttcactaggtaggaaatgtagaatttggttcctggc

SEQ ID NO: 329 - DNA, 38 bp, Artificial sequence
  Synthesized
tcacagggtgggagtcttaagtgttagctttcttgcag

SEQ ID NO: 330 - DNA, 38 bp, Artificial sequence
  Synthesized
acaggagactgggaaggtgctgtgctcgggactcagtg

SEQ ID NO: 331 - DNA, 50 bp, Artificial sequence
  Synthesized
taaagggctttggtgctgaatccatggtgaccgactttggaggtttaaca

SEQ ID NO: 332 - DNA, 50 bp, Artificial sequence
  Synthesized
acgtgatatctgggaatgtcctgcagatctcatgacaatactgacatctg

SEQ ID NO: 333 - DNA, 44 bp, Artificial sequence
  Synthesized
tctctcttctgtctattctgggccttcccagaagtggtggtcag

SEQ ID NO: 334 - DNA, 50 bp, Artificial sequence
  Synthesized
gccttgcccatttcgttcaactcttaggggctagcaactctagtatgttc

SEQ ID NO: 335 - skipped

SEQ ID NO: 336 - DNA, 16 bp, Artificial sequence
  GSTP1 reverse primer
cgaactcccgccgacc

SEQ ID NO: 337 - DNA, 15 bp, Artificial sequence
  GSTP1 probe
cgacgaccgctacac

SEQ ID NO: 338 - DNA, 19 bp, Artificial sequence
  SFRP2 forward primer
agtttttcggagttgcgcg

SEQ ID NO: 339 - DNA, 24 bp, Artificial sequence
  SFRP2 reverse primer
gctctcttcgctaaatacgactcg

SEQ ID NO: 340 - DNA, 17 bp, Artificial sequence
  SFRP2 probe
tgtagcgtttcgttcgc

SEQ ID NO: 341 - DNA, 22 bp, Artificial sequence
  IGFBP3 forward primer
ttttttcgatatcggttcgtcg

SEQ ID NO: 342 - DNA, 19 bp, Artificial sequence
  IGFBP3 reverse primer
gatctccttaaccccgccg

SEQ ID NO: 343 - DNA, 21 bp, Artificial sequence
  IGFBP3 probe
agattttatttcgagagcgga

SEQ ID NO: 344 - DNA, 17 bp, Artificial sequence
  IGFBP7 forward primer
aagcgggcgtgagatcg

SEQ ID NO: 345 - DNA, 19 bp, Artificial sequence
  IGFBP7 reverse primer
cgcgctcctactaacgtcg

SEQ ID NO: 346 - DNA, 19 bp, Artificial sequence
  IGFBP7 probe
ttatgggtcggttacgtcg

SEQ ID NO: 347 - DNA, 27 bp, Artificial sequence
  APC forward primer
ttatatgtccgttacgtgcgtttatat

SEQ ID NO: 348 - DNA, 19 bp, Artificial sequence
  APC reverse probe
gaaccaaaacgctccccat

SEQ ID NO: 349 - DNA, 22 bp, Artificial sequence
  APC probe
cccgtcgaaaacccgccgatta

SEQ ID NO: 350 - DNA, 19 bp, Artificial sequence
  PTGS2 forward primer
cggaagcgttcgggtaaag

SEQ ID NO: 351 - DNA, 20 bp, Artificial sequence
  PTGS2 reverse primer
gaattccaccgccccaaacg

SEQ ID NO: 352 - DNA, 29 bp, Artificial sequence
  PTGS2 probe
tttccgccaaatatcttttcttcttcgca

(19) United States
(12) Patent Application Publication
(10) Pub. No.: US 2024/0287612 A1
(43) Pub. Date: Aug. 29, 2024

(54) NOVEL BIOMARKERS AND DIAGNOSTIC PROFILES FOR PROSTATE CANCER INTEGRATING CLINICAL VARIABLES AND GENE EXPRESSION DATA

(71) Applicants: UEA ENTERPRISES LIMITED, Dublin 4 (IE); The University Of Surrey, Surrey (GB); The University Of Guildford, Surrey (GB)

(72) Inventors: Daniel Brewer, Norfolk (GB); Colin Cooper, Norfolk (GB); Jeremy Clark, Norfolk (GB); Shea Connell, Norfolk (GB); Antoinette Perry, Dublin (IE); Hardev Pandha, Surrey (GB)

(21) Appl. No.: 17/642,256

(22) PCT Filed: Sep. 14, 2020
(86) PCT No.: PCT/EP2020/075665

§ 371 Date: Mar. 11, 2022

Related U.S. Application Data
(60) Provisional application No. 62/899,328, filed on Sep. 12, 2019.

(30) Foreign Application Priority Data
Oct. 24, 2019 (GB) 1915464.0

(51) Int. Cl. C12Q 1/6886

(52) U.S. Cl. CPC C12Q 1/6886; C12Q 2600/118; C12Q 2600/154; C12Q 2600/158

(57) ABSTRACT

The present invention relates to prostate cancer (PC), in particular the use of biomarkers in biological samples for the diagnosis of such conditions, such as early stage prostate cancer. The present invention also relates to the use of biomarkers in biological samples for the classification of PC, and/or as a prognostic method for predicting the disease progression of prostate cancer.

### CROSS-REFERENCE TO RELATED APPLICATIONS

This application is the U.S. national phase application filed under 35 U.S.C. § 371 claiming benefit to International Patent Application No. PCT/EP2020/075665, filed on Sep. 14, 2020, which claims priority to U.S. Provisional Application No. 62/899,328, filed Sep. 12, 2019 and, Great Britain Patent Application No. 1915464.0, filed Oct. 24, 2019, the disclosures of which are incorporated herein by reference in their entirety.

### REFERENCE TO A “SEQUENCE LISTING”, A TABLE, OR A COMPUTER PROGRAM LISTING APPENDIX SUBMITTED AS AN ASCII TEXT FILE

The present application hereby incorporates by reference the entire contents of the text file named “206189-0042-00US_SequenceListingv2.txt” in ASCII format, which was created on Feb. 27, 2023, and is 65,713 bytes in size.

### FIELD OF THE INVENTION

The present invention relates to prostate cancer (PC), in particular the use of biomarkers in biological samples for the diagnosis of such conditions, such as early stage prostate cancer. The present invention also relates to the use of biomarkers in biological samples for the classification of PC, and/or as a prognostic method for predicting the disease progression of prostate cancer.

### INTRODUCTION

Prostate cancer exhibits extreme clinical heterogeneity; 10-year survival rates following diagnosis approach 84%, yet prostate cancer is still responsible for 13% of all cancer deaths in men in the UK [1]. Coupled with the high rates of diagnosis, prostate cancer is more often a disease that men die with rather than from. This illustrates the need for clinically implementable tools able to selectively identify those men that can be safely removed from treatment pathways without missing those men harbouring disease that requires intervention.

An opportune point to intervene or supplement current clinical practices would be prior to an initial biopsy in men suspected of having prostate cancer, reducing costs to men, healthcare systems and providers alike. In current clinical practice men are selected for further investigations for prostate cancer if they have an elevated PSA (≥4 ng/mL) and an adverse finding on digital rectal examination (DRE) or lower urinary tract symptoms; other factors such as age and ethnicity are also considered [2, 3, 4].

D'Amico stratification [5], which classifies patients as Low-Intermediate- or High-risk of PSA-failure post-radical therapy, is based on Gleason Score (Gs) [6], PSA and clinical stage, and has been used as a framework for guidelines issued in the UK, Europe and USA [7,8,9]. Low-risk, and some favourable Intermediate-risk patients are generally offered Active Surveillance (AS) while unfavourable Intermediate-, and High-risk patients are considered for radical therapy [7,10]. Other classification systems such as CAPRA score [11] use additional clinical information, assigning simple numeric values based on age, pre-treatment PSA, Gleason Score, percentage of biopsy cores positive for cancer and clinical stage for an overall 0-10 CAPRA score. The CAPRA score has shown favourable prediction of PSA-free survival, development of metastasis and prostate cancer-specific survival [12].

However, the rates of negative biopsies in men with a clinical suspicion of prostate cancer are overwhelming; a recent population-level study of 419,582 men from Martin et al observed that 60% of all biopsies in the control arm of the Cluster Randomized Trial of PSA Testing for Prostate Cancer (CAP) were negative for prostate cancer [13], similar to the rates observed by Donovan et al as part of the ProtecT trial [14]. Needle biopsy is invasive, and not without complications: 44% of patients report pain as a result of the biopsy, and detection of clinically insignificant disease can result in years of monitoring, causing patients undue stress [4]. Multiparametric MRI (MP-MRI) has been developed as a triage tool to reduce the rates of negative biopsy and its use has become increasingly widespread since its validation [15]. However, MP-MRI is relatively expensive and has shown a high rate of inter-operator and inter-machine variability, leading to mpMRI missing up to 28% of clinically significant diseases in practice [4, 16, 17, 18].

The interconnected nature of the male urological system makes it an ideal candidate for liquid biopsy and non-invasive biomarkers for prostate cancer. There is sizeable interest in the development of such non-invasive tests and classifiers capable of reducing the rates of initial biopsy in men, whilst retaining the sensitivity to detect aggressive disease. Single-gene or expression panels of few genes, such as the PCA3 [19], SelectMDx [20], ExoDx Prostate(IntelliScore) [21] tests have published promising results to date for the non-invasive detection of significant disease (Gleason score (Gs)≥7).

Similarly, several urine methylation panels have been developed; the ProCUrE assay from Zhao et al quantifies the methylation of HOXD4 and GSTP1 for the detection of CAPRA score 7-10 disease [22], whilst Brikun et al assessed the binary presence/absence of CpG island methylation associated with 18 genes to predict the presence of any prostate cancer on biopsy [23]. However, these biomarker panels have yet to be widely implemented in clinical settings, and none are currently recommended within the NICE guidelines [4], suggesting that improvements are required.

Other studies have aimed to detect the most aggressive cancers by utilising tissue samples taken at the time of biopsy, resulting in moderate success and wider clinical adoption [24,25,26]. However, due to their proposed implementation within current clinical pathways, these tests may not take into consideration the considerable economic, psychological and societal costs of unnecessarily subjecting men with low volume, indolent disease to biopsy [27, 28, 29].

In 2012, the Movember Global Action Plan 1 (GAP1) initiative was launched, a collaborative effort between multiple institutes focusing on prostate cancer biomarkers in urine, plasma, serum and extracellular vesicles. The prime aim of the GAP1 initiative was to develop a multi-modal urine biomarker panel forthe discrimination of disease state. The authors have previously published analyses from two of the GAP1 studies that measured differing molecular aspects within urine; epiCaPture assayed hypermethylation of urinary cell DNA [30], and PUR assessed transcript levels in cell-free extracellular vesicle mRNA (cf-RNA) using NanoString [31]. Both of these tests were able to discriminate some level of clinically significant disease and exhibited differing characteristics; where epiCaPture was well suited to detecting the highest grade disease (Gleason score ≥8), PUR was better matched to the deconvolution of lower risk and indolent disease, as detailed by its prognostic ability in active surveillance use.

With a suitable overlap in the numbers of patient samples analysed by both methods, we hypothesised that these two methods could be complementary, and the integration of both datasets could result in a more holistic model with predictive ability greater than the sum of its parts, able to encapsulate the clinical heterogeneity of prostate cancer and reach the levels of accuracy and utility required forwidespread adoption. In this study, we report the diagnostic accuracy of such an integrated model, determined by the ability to predict the presence of Gs≥7 and Gs≥4+3 disease on biopsy, both critical distinctions, where patients with Gs≥ 7 are recommended radical therapy [4], whilst patients with Gs 4+3 have significantly worse outcomes than Gs 3+4 patients [32]. Mindful that many cancer biomarkers fail to translate to the clinic, the development of the presented model has been carried out adhering to the transparent reporting of a multivariable prediction model for individual prognosis or diagnosis (TRIPOD) guidelines [33].

### SUMMARY OF THE INVENTION

Urine biomarkers offer the prospect of a more accurate assessment of cancer status prior to invasive tissue biopsy and may also be used to supplement standard clinical stratification using Gleason scores, Clinical Staging, PSA levels, and/or imaging techniques, such as magnetic resonance imaging (MRI).

In a first aspect of the invention, there is provided a method of providing a cancer diagnosis or prognosis based on one or more clinical variables and/or the expression status of a plurality of genes, comprising:
(a) providing a plurality of patient profiles each comprising the one or more clinical variables and/or the expression status of the plurality of genes in at least one sample obtained from each patient, wherein each of the patient profiles is associated with one of (n) biopsy outcome groups, wherein each biopsy outcome group is assigned a risk score and is associated with a different cancer prognosis or cancer diagnosis;
(b) applying a first supervised machine learning algorithm (for example random forest analysis) to the patient profiles to select a subset of one or more clinical variables and/or a subset of expression statuses of one or more genes from the plurality of genes in the patient profile that are associated with each biopsy outcome group;
(c) inputting the values of the subset of one or more clinical variables and/or subset of expression statuses of one or more genes into a second supervised machine learning algorithm (for example random forest analysis) comprising one or more decision trees;
(d) calculating a cut point for each of the one or more clinical variables and/or expression statuses of the one or more genes within the one or more decision trees to optimise the discrimination of each biopsy outcome group within the patient profiles, wherein the cut point can be used to generate a risk score for each decision tree;
(e) calculating an average risk score for each patient using the risk scores from each decision tree in (d); and
(f) providing a cancer diagnosis or prognosis for each patient or determining whether each patient has a poor prognosis based on whether the risk score for each patient is associated with a poor prognosis biopsy outcome group.

In a second aspect of the invention, there is provided a method of providing a cancer diagnosis or prognosis based on one or more clinical variables and/or the expression status of a plurality of genes, comprising:
(a) providing a reference dataset comprising a plurality of patient profiles each comprising the one or more clinical variables and expression status values of one or more genes in at least one sample obtained from each patient wherein the biopsy outcome group of each patient sample in the dataset is known and wherein each biopsy outcome group is assigned a risk score and is associated with a different cancer prognosis or cancer diagnosis;
(b) using the one or more clinical variables and/or expression status values for one or more genes to apply a supervised machine learning algorithm (for example random forest analysis) to the reference dataset to obtain a predictor for biopsy outcome group;
(c) determining the same one or more clinical variables and/or expression status values for the same one or more genes in a sample obtained from a test subject to provide a test subject profile; (d) applying the predictor to the test subject profile to generate a risk score for the test subject profile; and
(e) providing a cancer diagnosis or prognosis for the test subject or determining whether the test subject has a poor prognosis based on whether the risk score for the test subject profile is associated with a poor prognosis biopsy outcome group.

In a third aspect of the invention, there is provided a method of providing a cancer diagnosis or prognosis based on one or more clinical variables and/or the expression status of a plurality of genes, comprising:
(a) providing a reference dataset comprising a plurality of patient profiles each comprising the one or more clinical variables and expression status values of one or more genes in at least one sample obtained from each patient wherein the biopsy outcome group of each patient sample in the dataset is known and wherein each biopsy outcome group is assigned a risk score and is associated with a different cancer prognosis or cancer diagnosis;
(b) inputting the values of the one or more clinical variables and expression status values of one or more genes into a supervised machine learning algorithm (for example random forest analysis) comprising one or more decision trees;
(c) calculating a cut point for each of the one or more clinical variables and/or expression status of the one or more genes within the one or more decision trees to optimise the discrimination of each biopsy outcome group within the patient profiles, wherein the cut point can be used to generate a risk score for each decision tree;
(d) providing a test subject profile comprising values for the same one or more clinical variables and/or expression status of the same one or more genes in at least one sample obtained from the test subject;
(e) inputting the test subject profile into the supervised machine learning algorithm comprising the calculated cut points to generate a test subject risk score for each decision tree;
(f) calculating an average risk score for the test subject profile based on the risk scores for each decision tree calculated in step (e); and
(g) providing a cancer diagnosis or prognosis for the test subject or determining whether the test subject has a poor prognosis based on whether the average risk score for the test subject profile is associated with a poor prognosis biopsy outcome group.

In some embodiments of the second and third aspects of the invention, the one or more clinical variables and expression status values of one or more genes comprises the expression status of one or more of GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion and optionally PSA level (e.g. serum PSA level).

In some embodiments of the second and third aspects of the invention, the expression status of one or more of GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion is determined by methylation status. In a preferred embodiment of the second and third aspects of the invention, the expression status of one or more of GSTP1, APC, SFRP2, IGFBP3, IGFBP7, PTGS2 is determined by methylation status. In a further preferred embodiment of the second and third aspects of the invention, the expression status of all of GSTP1, APC, SFRP2, IGFBP3, IGFBP7, PTGS2 are determined by methylation status. In a preferred embodiment of the second and third aspects of the invention, the expression status of all of GSTP1, APC, SFRP2, IGFBP3, IGFBP7, PTGS2 are determined by methylation status and the expression status of ERG exons 4-5, ERG exons 6-7, GJB1, HOXC6, HPN, PCA3, SNORA20, TIMP4 and TMPRSS2/ERG fusion are determined by RNA microarray.

In some embodiments of the second and third aspects of the invention, the one or more clinical variables and expression status values of one or more genes comprises the expression status of one or more of EN2, ERG exons 4-5, ERG exons 6-7, GJB1, HOXC6, HPN, PCA3, PPFIA2, TMPRSS2/ERG fusion, SLC12A1 and TMEM45B fusion and optionally PSA level (e.g. serum PSA level).

In some embodiments of the second and third aspects of the invention, the expression status of one or more of EN2, ERG exons 4-5, ERG exons 6-7, GJB1, HOXC6, HPN, PCA3, PPFIA2, TMPRSS2/ERG fusion, SLC12A1 and TMEM45B fusion is determined by protein concentration in the sample. In a preferred embodiment of the second and third aspects of the invention, the expression status of EN2 is determined by protein concentration in the sample. In a preferred embodiment of the second and third aspects of the invention, the expression status of EN2 is determined by protein concentration in the sample and the expression status of ERG exons 4-5, ERG exons 6-7, GJB1, HOXC6, HPN, PCA3, PPFIA2, TMPRSS2/ERG fusion, SLC12A1 and TMEM45B fusion are determined by RNA microarray.

In a fourth aspect of the invention, there is provided a method of diagnosing or testing for prostate cancer in a subject comprising determining the expression status of one or more genes selected from the group consisting of GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion in a biological sample from the subject, optionally wherein the PSA level (e.g. serum PSA level) of the subject is also used in the method of diagnosing or testing for prostate cancer.

In a fifth aspect of the invention, there is provided a method of diagnosing or testing for prostate cancer in a subject comprising determining the expression status of one or more genes selected from the group consisting of EN2, ERG exons 4-5, ERG exons 6-7, GJB1, HOXC6, HPN, PCA3, PPFIA2, TMPRSS2/ERG fusion, SLC12A1 and TMEM45B fusion in a biological sample from the subject, optionally wherein the PSA level (e.g. serum PSA level) of the subject is also used in the method of diagnosing or testing for prostate cancer.

In some aspects of the invention the biopsy outcome group is classified by Gleason score (Gs). In some aspects of the invention the number of possible biopsy outcome groups (n) is 1, 2, 3, 4, 5, 6, 7, 8, 9 or 10.

In some aspects of the invention the n biopsy outcome groups comprise a group associated with no cancer diagnosis and one or more groups (e.g. 1, 2, 3 groups) associated with increasing risk of cancer diagnosis, severity of cancer or chance of cancer progression. In some aspects of the invention the higher a risk score is the higher the probability a given patient or test subject exhibits or will exhibit the clinical features or outcome of the corresponding biopsy outcome group.

In some aspects of the invention at least one of the biopsy outcome groups is associated with a poor prognosis of cancer. In some aspects of the invention the number of biopsy outcome groups (n) is 4. In a preferred aspect of the invention the 4 biopsy outcome groups are (i) no evidence of cancer, (ii) Gleason score (Gs)=6, (iii) Gleason score (Gs)=3+4 and (iv) Gleason score (Gs)≥4+3.

In some methods of the invention step (b) further comprises discarding any genes that are not associated with any of the n biopsy outcome groups.

In some aspects of the invention the one or more clinical variables and/or expression status of the plurality of genes is selected from one or more clinical variables and/or genes typically associated with the development of prostate cancer.

In some aspects of the invention, the biopsy outcome groups are classified based on a known clinical diagnosis, for example a biopsy outcome. In some aspects of the invention, the biopsy outcome groups can be cancer risk groups. In some aspects of the invention the biopsy outcome groups are classified by Gleason score, wherein patients with different ranges of Gleason scores are grouped into the same biopsy outcome group. In some aspects of the invention, the biopsy outcome groups can act as cancer classification groups.

In some aspects of the invention the association of each biopsy outcome group with a different cancer prognosis or cancer diagnosis corresponds to a known clinical diagnosis (for example a biopsy score on the Gleason scale) which can been provided as part of the patient profile. In some aspects of the invention, each patient profile in a reference or training dataset is associated with a biopsy outcome group based on a known clinical diagnosis (for example a biopsy score on the Gleason scale).

In some aspects of the invention the test subject profile does not comprise a known biopsy score or clinical classification.

In some aspects of the invention the one or more clinical variables and/or expression status of the plurality of genes is selected from the list in Table 1 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176 or 177 of the items in Table 1). In a preferred aspect the one or more clinical variables and/or expression status of the plurality of genes is all 177 variables listed in Table 1.

In some aspects of the invention the one or more clinical variables and/or expression status of the plurality of genes is selected from the list in the ExoRNA column of Table 1 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166 or 167 of the items in the ExoRNA column of Table 1). In a preferred aspect the one or more clinical variables and/or expression status of the plurality of genes is all 167 variables listed in the ExoRNA column of Table 1.

In some aspects of the invention the one or more clinical variables and/or expression status of the plurality of genes is selected from the list in the ExoMeth column of Table 1 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176 or 177 of the items in the ExoMeth column of Table 1). In a preferred aspect the one or more clinical variables and/or expression status of the plurality of genes is all 177 variables listed in the ExoMeth column of Table 1.

In some aspects of the invention the one or more clinical variables and/or expression status of the plurality of genes is selected from the list in the ExoGrail column of Table 1 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171 or 172 of the items in the ExoGrail column of Table 1). In a preferred aspect the one or more clinical variables and/or expression status of the plurality of genes is all 172 variables listed in the ExoGrail column of Table 1.

In some aspects of the invention the subset of one or more clinical variables and/or expression status of the plurality of genes is selected from the list of genes in the ExoMeth column of Table 3 (i.e. 1, 2, 3, 4, 5, 6, 7, 8, 9, 10, 11, 12, 13, 14, 15 or 16 of the genes in Table 3). In a preferred embodiment, the subset of one or more clinical variables and/or expression status of the plurality of genes is all 16 variables listed the ExoMeth column of Table 3.

In some aspects of the invention the subset of one or more clinical variables and/or expression status of the plurality of genes is selected from the list of genes in the ExoGrail column of Table 5 (i.e. 1, 2, 3, 4, 5, 6, 7, 8, 9, 10, 11 or 12 of the genes in Table 5). In a preferred embodiment, the subset of one or more clinical variables and/or expression status of the plurality of genes is all 12 variables listed the ExoGrail column of Table 3.

In some aspects of the invention the expression status of one or more genes is determined by methylation status, optionally wherein the expression status of one or more of GSTP1, APC, SFRP2, IGFBP3, IGFBP7 and PTGS2 is determined by methylation status.

In some aspects of the invention the expression status of one or more genes is determined by protein quantification, optionally wherein the expression status of EN2 is determined by protein quantification. In a preferred aspect of the invention the expression status of one or more genes is determined by protein ELISA.

In a preferred aspect of the invention the method can be used to determine whether a patient should be biopsied. In some aspects of the invention the method is used in combination with MRI imaging data to determine whether a patient should be biopsied. In some aspects of the invention the MRI imaging data is generated using multiparametric MRI (MP MRI). In some aspects of the invention the MRI imaging data is used to generate a Prostate Imaging Reporting and Data System (PI RADS) grade. In some aspects of the invention the method can be used to predict disease progression in a patient. In some aspects of the invention the patient is currently undergoing or has been recommended for active surveillance.

In some aspects of the invention the patient is currently undergoing active surveillance by PSA monitoring, biopsy and repeat biopsy and/or MRI, at least every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks or 24 weeks. In some aspects of the invention the method can be used to predict disease progression in patients with a Gleason score of ≤10, ≤9, ≤8, ≤7 or ≤6. In some aspects of the invention the method can be used to predict:
(i) the volume of Gleason 4 or Gleason ≥4 prostate cancer; and/or
(ii) low risk disease that will not require treatment for 1, 2, 3, 4, 5 or more years.

In some aspects of the invention the biological sample is processed prior to determining the expression status of the one or more genes in the biological sample. In some aspects of the invention determining the expression status of the one or more genes comprises extracting RNA from the biological sample. In some aspects of the invention the RNA is extracted from extracellular vesicles.

In some aspects of the invention determining the expression status of the one or more genes comprises the step of quantifying the expression status of the RNA transcript or cDNA molecule and wherein the expression status of the RNA or cDNA is quantified using any one or more of the following techniques: microarray analysis, real time quantitative PCR, DNA sequencing, RNA sequencing, Northern blot analysis, in situ hybridisation and/or detection and quantification of a binding molecule. In some aspects of the invention determining the expression status of the RNA or cDNA comprises RNA or DNA sequencing. In some aspects of the invention determining the expression status of the RNA or cDNA comprises using a microarray.

In some aspects of the invention the microarray detection further comprises the step of capturing the one or more RNAs or cDNAs on a solid support and detecting hybridisation. In some aspects of the invention the microarray detection further comprises sequencing the one or more RNA or cDNA molecules.

In some aspects of the invention the microarray comprises a probe having a nucleotide sequence with at least 80%, 85%, 90%, 95%, 96%, 97%, 98% or 99% identity to a nucleotide sequence selected from any one of SEQ ID NOs 1 to 334. In some aspects of the invention the microarray comprises a probe having a nucleotide sequence selected from any one of SEQ ID NOs 1 to 334. In some aspects of the invention the microarray comprises 334 probes each having a nucleotide sequence with at least 80%, 85%, 90%, 95%, 96%, 97%, 98% or 99% identity to a unique nucleotide sequence selected from any one of SEQ ID NOs 1 to 334. In some aspects of the invention the microarray comprises 334 probes, each having a unique nucleotide sequence selected from SEQ ID NOs 1 to 334.

In some aspects of the invention the microarray comprises a pair of probes having a nucleotide sequence with at least 80%, 85%, 90%, 95%, 96%, 97%, 98% or 99% identity to a pair of nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 277 and SEQ ID NO: 278, and SEQ ID NO: 313 and SEQ ID NO: 314.

In some aspects of the invention the microarray comprises a pair of probes for every gene of interest having nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 277 and SEQ ID NO: 278, and SEQ ID NO: 313 and SEQ ID NO: 314.

In some aspects of the invention the microarray comprises a pair of probes having a nucleotide sequence with at least 80%, 85%, 90%, 95%, 96%, 97%, 98% or 99% identity to a pair of nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 219 and SEQ ID NO: 220, SEQ ID NO: 265 and SEQ ID NO: 266, and SEQ ID NO: 317 and SEQ ID NO: 318.

In some aspects of the invention the microarray comprises a pair of probes for every gene of interest having nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 219 and SEQ ID NO: 220, SEQ ID NO: 265 and SEQ ID NO: 266, and SEQ ID NO: 317 and SEQ ID NO: 318.

In some aspects of the invention determining the expression status of the one or more genes comprises extracting protein from the biological sample. In some aspects of the invention the protein is extracted directly from the biological sample.

In some aspects of the invention determining the expression status of the one or more genes comprises determining the methylation status of one or more genes. In some aspects of the invention the method further comprises a step of comparing or normalising the expression status of one or more genes with the expression status of a reference gene.

In some aspects of the invention the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample). In a preferred aspect of the invention the biological sample is a urine sample. In a preferred aspect of the invention the sample is from a human.

In an sixth aspect of the invention, there is provided a method of treating prostate cancer, comprising diagnosing a patient as having or as being suspected of having prostate cancer using a diagnostic method of the invention and administering to the patient a therapy for treating prostate cancer.

In a seventh aspect of the invention, there is provided a method of treating prostate cancer in a patient, wherein the patient has been determined as having prostate cancer or as being suspected of having prostate cancer according to a diagnostic method of the invention, comprising administering to the patient a therapy for treating prostate cancer.

In some aspects of the invention the therapy for prostate cancer comprises surgery, brachytherapy, active surveillance, chemotherapy, hormone therapy, immunotherapy and/or radiotherapy. In some aspects of the invention the chemotherapy comprises administration of one or more agents selected from the following list: abiraterone acetate, apalutamide, bicalutamide, cabazitaxel, bicalutamide, degarelix, docetaxel, leuprolide acetate, enzalutamide, apalutamide, flutamide, goserelin acetate, mitoxantrone, nilutamide, sipuleucel T, radium 223 dichloride and docetaxel.

In some aspects of the invention the therapy for prostate cancer comprises resection of all or part of the prostate gland or resection of a prostate tumour.

In a eighth aspect of the invention, there is provided an RNA, DNA, cDNA or protein molecule of one or more genes selected from the group consisting of: GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion for use in a method of diagnosing or testing for prostate cancer comprising determining the expression status of the one or more genes, optionally wherein the PSA level (e.g. serum PSA level) of the subject is also used in the method of diagnosing or testing for prostate cancer.

In some aspects of the invention the expression status of one or more genes is determined by methylation status, optionally wherein the expression status of one or more of GSTP1, APC, SFRP2, IGFBP3, IGFBP7 and PTGS2 is determined by methylation status.

In an ninth aspect of the invention, there is provided an RNA, DNA, cDNA or protein molecule of one or more genes selected from the group consisting of EN2, ERG exons 4-5, ERG exons 6-7, GJB1, HOXC6, HPN, PCA3, PPFIA2, TMPRSS2/ERG fusion for use in a method of diagnosing or testing for prostate cancer comprising determining the expression status of the one or more genes, optionally wherein the PSA level (e.g. serum PSA level) of the subject is also used in the method of diagnosing or testing for prostate cancer.

In some aspects of the invention the expression status of one or more genes is determined by protein quantification, optionally wherein the expression status of EN2 is determined by protein quantification, further optionally wherein the expression status is determined by protein ELISA.

In a tenth aspect of the invention there is provided a kit for testing for prostate cancer comprising a means for measuring the expression status of:
(i) one or more genes selected from the group consisting of: GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion; or
(ii) one or more genes selected from the group consisting of: EN2, ERG exons 4-5, ERG exons 6-7, GJB1, HOXC6, HPN, PCA3, PPFIA2, TMPRSS2/ERG fusion, in a biological sample, optionally wherein the kit further comprises a means for measuring PSA level (e.g. serum PSA level).

In some kits of the invention the expression status of one or more genes is determined by methylation status, optionally wherein the expression status of one or more of GSTP1, APC, SFRP2, IGFBP3, IGFBP7 and PTGS2 is determined by methylation status.

In some kits of the invention the expression status of one or more genes is determined by protein quantification, optionally wherein the expression status of EN2 is determined by protein quantification, further optionally wherein the expression status is determined by protein ELISA.

In a eleventh aspect of the invention there is provided a kit of parts for providing a cancer diagnosis or prognosis based on one or more clinical variables and/or the expression status of a plurality of genes, comprising a means for quantifying biomarkers, such as the expression status of one or more gene transcripts, methylation status of one or more genes, and/or the concentration of (i.e. measuring) one or more proteins selected from the group consisting of GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion, optionally wherein the kit further comprises a means for measuring PSA level (e.g. serum PSA level).

The means may be any suitable detection means that can measure the quantity or expression status of biomarkers in the sample. In some embodiments of the invention, the expression status, methylation status or concentration of one or more biomarkers can be combined with one or more clinical parameters (such as PSA level (e.g. serum PSA level), age at sample collection, DRE impression and urine volume collected) to provide a cancer diagnosis or prognosis. In a preferred embodiment the expression status, methylation status or concentration of one or more biomarkers can be combined with PSA level (e.g. serum PSA level) to provide a cancer diagnosis or prognosis.

In a preferred embodiment of the invention the methylation status of one or more of GSTP1, APC, SFRP2, IGFBP3, IGFBP7 and PTGS2 can be used to provide a prostate cancer diagnosis or prognosis. In a preferred embodiment, the invention provides a kit of parts for providing a prostate cancer diagnosis or prognosis comprising a means for quantifying the methylation status of one or more of GSTP1, APC, SFRP2, IGFBP3, IGFBP7 and PTGS2 and the transcript levels of one or more of ERG exons 4-5, ERG exons 6-7, GJB1, HOXC6, HPN, PCA3, SNORA20, TIMP4 and TMPRSS2/ERG fusion, optionally wherein the kit further comprises a means for measuring PSA level (e.g. serum PSA level).

In a still further embodiment of the invention there is provided a kit of parts for providing a prostate cancer diagnosis or prognosis comprising a means for quantifying biomarkers, such as the expression status of one or more gene transcripts, methylation status of one or more genes, and/or the concentration of (i.e. measuring) one or more proteins selected from the group consisting of EN2, ERG exons 4-5, ERG exons 6-7, GJB1, HOXC6, HPN, PCA3, PPFIA2, TMPRSS2/ERG fusion, SLC12A1 and TMEM45B, optionally wherein the kit further comprises a means for measuring PSA level (e.g. serum PSA level).

The means may be any suitable detection means that can measure the quantity of biomarkers in the sample.

In some embodiments of the invention, the expression status, methylation status or concentration of one or more gene transcripts can be combined with one or more clinical parameters (such as PSA level (e.g. serum PSA level), age at sample collection, DRE impression and urine volume collected) to provide a cancer diagnosis or prognosis. In a preferred embodiment the expression status, methylation status or concentration of one or more gene transcripts can be combined with PSA level (e.g. serum PSA level) to provide a cancer diagnosis or prognosis.

In a preferred embodiment the protein concentration (as established by ELISA, for example) of EN2 can be used to provide a cancer diagnosis or prognosis. In a preferred embodiment, the invention provides a kit of parts for providing a prostate cancer diagnosis or prognosis comprising a means for quantifying the protein concentration of EN2 and the transcript levels of one or more of ERG exons 4-5, ERG exons 6-7, GJB1, HOXC6, HPN, PCA3, PPFIA2, TMPRSS2/ERG fusion, SLC12A1 and TMEM45B, optionally wherein the kit further comprises a means for measuring PSA level (e.g. serum PSA level).

In one embodiment, the means may be a biosensor. The kit may also comprise a container for the sample or samples and/or a solvent for extracting the biomarkers from the biological sample. The kits of the present invention may also comprise instructions for use.

The kit of parts of the invention may comprise a biosensor. A biosensor incorporates a biological sensing element and provides information on a biological sample, for example the presence (or absence) or concentration of an analyte. Specifically, they combine a biorecognition component (a bioreceptor) with a physiochemical detector for detection and/or quantification of an analyte (such as an RNA, a cDNA or a protein).

The bioreceptor specifically interacts with or binds to the analyte of interest and may be, for example, an antibody or antibody fragment, an enzyme, a nucleic acid, an organelle, a cell, a biological tissue, imprinted molecule or a small molecule. The bioreceptor may be immobilised on a support, for example a metal, glass or polymer support, or a 3-dimensional lattice support, such as a hydrogel support.

Biosensors are often classified according to the type of biotransducer present. For example, the biosensor may be an electrochemical (such as a potentiometric), electronic, piezoelectric, gravimetric, pyroelectric biosensor or ion channel switch biosensor. The transducer translates the interaction between the analyte of interest and the bioreceptor into a quantifiable signal such that the amount of analyte present can be determined accurately. Optical biosensors may rely on the surface plasmon resonance resulting from the interaction between the bioreceptor and the analyte of interest. The SPR can hence be used to quantify the amount of analyte in a test sample. Other types of biosensor include evanescent wave biosensors, nanobiosensors and biological biosensors (for example enzymatic, nucleic acid (such as DNA), antibody, epigenetic, organelle, cell, tissue or microbial biosensors).

The invention also provides microarrays (RNA, DNA or protein) comprising capture molecules (such as RNA or DNA oligonucleotides) specific for each of the biomarkers or biomarker panels being quantified, wherein the capture molecules are immobilised on a solid support. The microarrays are useful in the methods of the invention.

The binding molecules may be present on a solid substrate, such an array (for example an RNA microarray, in which case the binding molecules are DNA or RNA molecules that hybridise to the target RNA or cDNA). The binding molecules may all be present on the same solid substrate. Alternatively, the binding molecules may be present on different substrates. In some embodiments of the invention, the binding molecules are present in solution.

These kits may further comprise additional components, such as a buffer solution. Other components may include a labelling molecule for the detection of the bound RNA and so the necessary reagents (i.e. enzyme, buffer, etc) to perform the labelling; binding buffer; washing solution to remove all the unbound or non-specifically bound RNAs. Hybridisation will be dependent on the size of the putative binder, and the method used may be determined experimentally, as is standard in the art. As an example, hybridisation can be performed at ˜20° C. below the melting temperature (Tm), over-night. (Hybridisation buffer: 50% deionised formamide, 0.3 M NaCl, 20 mM Tris-HCl, pH 8.0, 5 mM EDTA, 10 mM phosphate buffer, pH 8.0, 10% dextran sulfate, 1×Denhardt's solution, and 0.5 mg/mL yeast tRNA). Washes can be performed at 4-6° C. higher than hybridisation temperature with 50% Formamide/2×SSC (20× Standard Saline Citrate (SSC), pH 7.5: 3 M NaCl, 0.3 M sodium citrate, the pH is adjusted to 7.5 with 1 M HCl). A second wash can be performed with 1×PBS/0.1% Tween 20.

Binding or hybridisation of the binding molecules to the target analyte may occur under standard or experimentally determined conditions. The skilled person would appreciate what stringent conditions are required, depending on the biomarkers being measured. The stringent conditions may include a hybridisation buffer that is high in salt concentration, and a temperature of hybridisation high enough to reduce non-specific binding.

In some kits of the invention the means for detecting is a biosensor or specific binding molecule. In some kits of the invention the biosensor is an electrochemical, electronic, piezoelectric, gravimetric, pyroelectric biosensor, ion channel switch, evanescent wave, surface plasmon resonance or biological biosensor. In some kits of the invention the means for detecting the expression status of the one or more genes is a microarray. In some kits of the invention the means for detecting the expression status of the one or more genes is an ELISA.

In some kits of the invention the kit comprises multiple means for detecting the expression status of the one or more genes. In some kits of the invention the multiple means for detecting the expression status of the one or more genes is a microarray and an ELISA. In some kits of the invention the multiple means for detecting the expression status of the one or more genes is multiple microarrays (e.g. an expression microarray and a methylation microarray).

In some kits of the invention the microarray comprises specific probes that hybridise to one or more genes selected from the group consisting of: GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion. In some kits of the invention the microarray comprises specific probes that hybridise to one or more genes selected from the group consisting of: EN2, ERG exons 4-5, ERG exons 6-7, GJB1, HOXC6, HPN, PCA3, PPFIA2, TMPRSS2/ERG fusion.

In some kits of the invention the microarray comprises a probe having a nucleotide sequence with at least 80%, 85%, 90%, 95%, 96%, 97%, 98% or 99% identity to a nucleotide sequence selected from any one of SEQ ID NOs 1 to 334. In some kits of the invention the microarray comprises a probe having a nucleotide sequence selected from any one of SEQ ID NOs 1 to 334. In some kits of the invention the microarray comprises 334 probes each having a nucleotide sequence with at least 80%, 85%, 90%, 95%, 96%, 97%, 98% or 99% identity to a unique nucleotide sequence selected from any one of SEQ ID NOs 1 to 334. In some kits of the invention the microarray comprises 334 probes, each having a unique nucleotide sequence selected from SEQ ID NOs 1 to 334.

In some kits of the invention the microarray comprises a pair of probes having a nucleotide sequence with at least 80%, 85%, 90%, 95%, 96%, 97%, 98% or 99% identity to a pair of nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 277 and SEQ ID NO: 278, and SEQ ID NO: 313 and SEQ ID NO: 314. In some kits of the invention the microarray comprises a pair of probes for every gene of interest having nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 277 and SEQ ID NO: 278, and SEQ ID NO: 313 and SEQ ID NO: 314. In some kits of the invention the microarray comprises a pair of probes having a nucleotide sequence with at least 80%, 85%, 90%, 95%, 96%, 97%, 98% or 99% identity to a pair of nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 219 and SEQ ID NO: 220, SEQ ID NO: 265 and SEQ ID NO: 266, and SEQ ID NO: 317 and SEQ ID NO: 318. In some kits of the invention the microarray comprises a pair of probes for every gene of interest having nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 219 and SEQ ID NO: 220, SEQ ID NO: 265 and SEQ ID NO: 266, and SEQ ID NO: 317 and SEQ ID NO: 318.

In some kits of the invention the kit further comprises one or more solvents for extracting RNA and/or protein from the biological sample.

In a further aspect of the invention there is provided a computer apparatus configured to perform a method of the invention. In a fourteenth aspect of the invention there is provided a computer readable medium programmed to perform a method of the invention. In some kits of the invention the kit further comprises a computer readable medium programmed to perform a method of the invention.

In a further aspect the invention provides a method of providing a cancer diagnosis or prognosis based on one or more clinical variables and/or the expression status of a plurality of genes comprising determining the methylation status of one or more genes selected from the group consisting of GSTP1, APC, SFRP2, IGFBP3, IGFBP7, PTGS2, and the expression status of one or more genes selected from the group consisting of f ERG exons 4-5, ERG exons 6-7, GJB1, HOXC6, HPN, PCA3, SNORA20, TIMP4 and TMPRSS2/ERG fusion in a biological sample from the subject, optionally wherein the serum PSA level of the subject is also used in the method of diagnosing or testing for prostate cancer.

In a further aspect the invention provides a method of providing a cancer diagnosis or prognosis based on one or more clinical variables and/or the expression status of a plurality of genes comprising determining the expression status of EN2 by protein quantification and the expression of one or more genes selected from the group consisting of ERG exons 4-5, ERG exons 6-7, GJB1, HOXC6, HPN, PCA3, PPFIA2, TMPRSS2/ERG fusion, SLC12A1 and TMEM45B fusion in a biological sample from the subject, optionally wherein the serum PSA level of the subject is also used in the method of diagnosing or testing for prostate cancer.

In a further aspect of the invention, there is provided a method of providing a cancer diagnosis or prognosis based on one or more clinical variables and/or the expression status of one or more genes comprising:
(a) providing a reference dataset wherein the biopsy outcome group of each patient sample in the dataset is known;
(b) using the one or more clinical variables and/or the expression status of one or more genes to apply a supervised machine learning algorithm on the dataset to obtain a predictor for biopsy outcome group;
(c) providing or determining the same one or more clinical variables and/or the expression status of the same one or more genes in a sample obtained from a test subject to provide a test subject profile;
(d) applying the predictor to the test subject profile to classify the cancer, or to predict the biopsy outcome group of the test subject.

In some aspects of the invention the expression status of one or more genes is determined by one or more methods including, protein quantification, methylation status, RNA extraction, RNA hybridisation or sequencing, optionally wherein the expression status of EN2 is determined by protein quantification.

In some aspects of the invention calculating an average risk score involves generating the mean, median or modal value of the risk scores generated by each decision tree. In a preferred embodiment, calculating an average risk score involves generating the mean value of the risk scores generated by each decision tree.

In some aspects of the invention the one or more clinical variables can include one or more quantitative parameters typically associated with the diagnosis or monitoring of patients suspected of or having prostate cancer. In some aspects of the invention the one or more clinical variables can include one or more of PSA level (e.g. serum PSA level), urine volume, age and/or prostate size, as assessed by digital rectal examination (DREsize). In a preferred embodiment of the invention, the clinical variable includes PSA level (e.g. serum PSA level).

In some aspects of the invention providing a cancer diagnosis or prognosis or determining whether the patient or test subject has a poor prognosis comprises comparing the average risk value generated by the predictor or supervised machine learning algorithm with the risk values assigned to the biopsy outcome groups and assessing whether the average risk score is more closely aligned with risk scores assigned to higher-risk biopsy outcome groups or lower-risk biopsy outcome groups. In some aspects of the invention “higher risk” and “lower risk” refer to the risk of a patient or test subject having or developing prostate cancer. For example, if three biopsy outcome groups (low-, medium- and high-risk) are assigned values of 0, 0.5 and 1 for the purposes of generating a predictor or applying a supervised machine learning algorithm then a patient or test subject with an average risk score of 0.75 would have a cancer diagnosis or prognosis corresponding to between medium- and high-risk. In the same example, a patient or test subject with an average risk score of 0.9 would have a cancer diagnosis or prognosis corresponding to a higher-risk and a patient or test subject with an average risk score of 0.2 would have a cancer diagnosis or prognosis corresponding to a lower-risk.

In some aspects of the invention selecting a subset of one or more clinical variables and/or expression status of one or more genes comprises using a random forest classifier applied to a training or reference dataset, wherein the training or reference dataset comprises shadow features generated by randomly shuffling the dataset for each variable. The random forest classifier can compare each of the input features against the shadow features and select only those which are important for classifying the patient profiles. In some aspects of the invention feature selection is conducted using the Boruta algorithm.

In some aspects of the invention selecting a subset of one or more clinical variables and/or expression status of one or more genes from the plurality of genes in the patient profile that are associated with each biopsy outcome group comprises applying a supervised machine learning algorithm (for example a random forest analysis, such as the Boruta algorithm) constrained with a predefined set of criteria for determining feature significance. In some aspects of the invention, the predefined set of criteria can comprise a predefined number of iterations (or resamples) and/or a predefined proportion of iterations (or resamples) in which a feature must be selected. In some aspects of the invention, the predefined number of iterations is 1000 and/or the predefined proportion of iterations (or resamples) in which a feature must be selected to be considered associated with a biopsy outcome group is 90%. In a preferred embodiment of the invention the predefined number of iterations is 1000 and the predefined proportion of iterations (or resamples) in which a feature must be selected to be considered associated with a biopsy outcome group is 90%.

In some aspects of the invention a resample is a new random selection of the original dataset which is constructed by randomly drawing observations/samples from the original dataset one at a time and returning them to the original dataset after they have been chosen until the size of the new and original dataset are the same.

In some aspects of the invention, calculating a cut point for each of the one or more clinical variables and/or expression statuses of the one or more genes within the one or more decision trees is based on the values of the one or more clinical variables and/or expression statuses of the one or more genes. In some aspects of the invention, the values of the one or more clinical variables and/or expression statuses of the one or more genes are provided in the same units in the patient profiles and in the test subject profile (for example age in years). In some aspects of the invention, the values of the one or more clinical variables and/or expression statuses of the one or more genes are provided in the same units in the reference dataset and in the test subject profile. In some aspects of the invention, the values of the one or more clinical variables and/or expression statuses of the one or more genes are numerical values. In some aspects of the invention, the values of the one or more clinical variables and/or expression statuses of the one or more genes are continuous values (i.e. not discrete). In some aspects of the invention, the values of the one or more clinical variables and/or expression statuses of the one or more genes are continuous numerical values.

Supervised machine learning algorithms or general linear models are used to produce a predictor of cancer risk. The preferred approach is random forest analysis but alternatives such as support vector machines, neural networks or naive Bayes classifier could be used. Such methods are known and understood by the skilled person.

Random forest analysis can be used to predict whether a patient profile (comprising one or more clinical variables such as PSA level (e.g. serum PSA level), gene expression data, gene methylation data and/or protein concentration data) is associated with a particular biopsy outcome group.

A random forest analysis is an ensemble learning method for classification, regression and other tasks, which operates by constructing a multitude of decision trees during training and outputting the class that is the mode of the classes (classification) or mean prediction (regression) of the decision trees. Accordingly, a random forest corrects for overfitting of data to any one decision tree.

A decision tree comprises a tree-like graph or model of decisions and their possible consequences, including chance event outcomes. Each internal node of a decision tree typically represents a test on an attribute or multiple attributes (for example whether an expression level of a gene in a cancer sample is above a predetermined threshold), each branch of a decision tree typically represents an outcome of a test, and each leaf node of the decision tree typically represents a class (classification) label or value along a continuous scale (regression).

In a random forest analysis, an ensemble classifier is typically trained on a training dataset (also referred to as a reference dataset) wherein the biopsy outcome group for each patient profile of the training dataset is known. The training produces a model that is a predictor for membership of each biopsy outcome group or the average predicted value in the case of regression trees. Once trained the random forest classifier can then be applied to a dataset from an unknown sample. This step is deterministic i.e. if the classifier is subsequently applied to the same dataset repeatedly, it will consistently sort each cancer of the new dataset into the same class each time.

In a preferred embodiment of the invention, a predictor is a trained random forest based algorithm which has been provided with a reference dataset comprising a plurality of patient profiles each comprising one or more clinical variables and expression status values of one or more genes in at least one sample obtained from each patient wherein the biopsy outcome group of each patient sample in the dataset is known and wherein each biopsy outcome group is assigned a risk score and is associated with a different cancer prognosis or cancer diagnosis.

When the random forest analysis is undertaken, the ensemble classifier splits the patient profiles in the dataset being analysed into a number of classes, each associated with a biopsy outcome group in the training or reference dataset. The number of groups may be 2, 3, 4, 5, 6, 7, 8, 9, 10 or more (e.g. the biopsy outcome groups may be associated with different Gleason scores, for example wherein there are four groups associated with (i) no evidence of cancer, (ii) Gs=6, (iii) Gs=3+4 and (iv) Gs≥ 4+3). In the present case these groups are treated as being along a continuum, that is where any value between the individual groups can also exist.

Each decision tree in the random forest is an independent predictor that, given a patient profile, provides a risk score (a score along a single continuous variable) for each of the classes which it has been trained to recognize, (e.g. no evidence of cancer, (ii) Gs=6, (iii) Gs=3+4 and (iv) Gs≥ 4+3). Each node of each decision tree comprises a test concerning one or more genes of the same plurality of genes as obtained in the patient profile from the patient. Several genes may be tested at the node. For example, a test may ask whether the expression level(s) of one or more genes of the plurality of genes is above a predetermined threshold.

Variations between decision trees will lead to each decision tree assigning a sample a score or class in a different way. The ensemble classifier takes the classification produced by all the independent decision trees and assigns the sample to the class on which the most decision trees agree (classification) or mean prediction of the individual decision trees (regression).

The reference dataset may have been obtained previously and, in general, the obtaining of these datasets is not part of the claimed method. However, in some embodiments, the method may further comprise obtaining the additional datasets for inclusion in the analysis. The reference dataset is in the form of a plurality of patient profiles (i.e. one or more clinical variables and/or one or more expression status values) that comprise the same variables measured in the test subject sample.

### BRIEF DESCRIPTION OF THE FIGURES

FIG. 1—Boruta analysis of variables available for the training of the ExoMeth model. Variable importance was determined over 1,000 bootstrap resamples of the available data and the decision reached recorded at each resample. Colour indicates the proportion of the 1,000 resamples a variable was confirmed to be important in. Variables confirmed in at least 90% of resamples were selected for predictive modelling. Those variables rejected in every single resample are not shown here, but the full list of inputs for all models can be seen in Table 1.

FIG. 2—Waterfall plot of the ExoMeth risk score for each patient. Each coloured bar represents an individual patient's calculated risk score and their true biopsy outcome, coloured according to Gleason score (Gs). Green—No evidence of cancer, Blue—Gs 6, Orange—Gs 3+4, Red—Gs≥ 4+3.

FIG. 3—Density plots detailing risk score distributions generated from four trained models. Models A to D were trained with different input variables; A: SoC clinical risk model, including Age and PSA, B: Methylation model, C: ExoRNA model and D: ExoMeth model, combining the predictors from all three previous models. The full list of variables in each model is available in Table 1. Fill colour shows the risk score distribution of patients with a significant biopsy outcome of Gs≥ 3+4 (Orange) or Gs≤6 (Blue).

FIG. 4—Cumming estimation plot of the ExoMeth risk signature. The top row details individual patients as points, separated according to Gleason score on the x-axis and risk score on the y-axis. Points are coloured according to clinical risk category; NEC—No evidence of cancer, Raised PSA—Raised PSA with negative biopsy, L-D'Amico Low-Risk, I—D'Amico Intermediate Risk, H—D'Amico High-Risk. Gapped vertical lines detail the mean and standard deviation of each group's risk scores. The lower panel shows the mean differences in risk score of each group, as compared to the NEC samples. Mean differences and 95% confidence interval are displayed as a point estimate and vertical bar respectively, using the sample density distributions calculated from a bias-corrected and accelerated bootstrap analysis from 1,000 resamples.

FIG. 5—Decision curve analysis (DCA) plots detailing the standardised net benefit (sNB) of adopting different risk models for aiding the decision to biopsy patients who present with a PSA≥4 ng/mL. The x-axis details the range of risk a clinician or patient may accept before deciding to biopsy. Panels show the sNB based upon the detection of varying levels of disease severity: A: detection of Gleason ≥4+3, B: detection of Gleason ≥3+4, C—any cancer; Blue-biopsy all patients with a PSA>4 ng/mL, Orange—biopsy patients according to the SOC model, Green—biopsy patients based on the methylation model, Purple—biopsy patients based on the ExoRNA model, Red—biopsy patients based on a the ExoMeth model. To assess the benefit of adopting these risk models in a non-PSA screened population we used data available from the control arm of the CAP study [13]. DCA curves were calculated from 1,000 bootstrap resamples of the available data to match the distribution of disease reported in the CAP trial population. Mean sNB from these resampled DCA results are plotted here.

FIG. 6—Net percentage reduction in biopsies, as calculated by DCA measuring the benefit of adopting different risk models for aiding the decision to biopsy patients who would otherwise undergo biopsy by current clinical guidelines. The x-axis details the range of accepted risk a clinician or patient may accept before deciding to biopsy. Panels show the reduction in biopsies per 100 patients based upon the detection of varying levels of disease severity: A: detection of Gleason ≥4+3, B: detection of Gleason ≥3+4 and C—any cancer. Coloured lines show differing comparator models; Blue-biopsy all patients with a PSA>3 ng/mL, Orange—biopsy patients by according the to the SoC model, Green—biopsy patients based on the methylation model, Purple—biopsy patients based on the ExoRNA model, Red—biopsy patients based on a the ExoMeth model. To assess the benefit of adopting these risk models in a non-PSA screened population we used data available from the control arm of the CAP study [13]. DCA curves were calculated from 1,000 bootstrap resamples of the available data to match the distribution of disease reported in the CAP trial population. Mean sNB from these resampled DCA results are used to calculate the potentially reductions in biopsy rates here.

FIG. 7—Boruta analysis of variables available for the training of the SoC model. Variable importance was determined over 1,000 bootstrap resamples of the available data and the decision reached recorded at each resample. Variable origins are denoted by font; clinical variables are italicised and emboldened. Colour indicates the proportion of the 1,000 resamples a variable was confirmed to be important in. Variables confirmed in at least 90% of resamples were selected for training predictive models.

FIG. 8—Boruta analysis of variables available for the training of the Methylation model. Variable importance was determined over 1,000 bootstrap resamples of the available data and the decision reached recorded at each resample. Variable origins are denoted by font; methylation variables are italicised. Colour indicates the proportion of the 1,000 resamples a variable was confirmed to be important in. Variables confirmed in at least 90% of resamples were selected for training predictive models.

FIG. 9—Boruta analysis of variables available for the training of the ExoRNA model (ExoMeth comparator). Variable importance was determined over 1,000 bootstrap resamples of the available data and the decision reached recorded at each resample. Variable origins are denoted by font; clinical variables are emboldened. Colour indicates the proportion of the 1,000 resamples a variable was confirmed to be important in. Variables confirmed in at least 90% of resamples were selected for training predictive models. Those variables rejected in every single resample are not shown here, but the full list of inputs for the ExoRNA model can be seen in Table 1.

FIG. 10—Density plots detailing risk score distributions generated from four trained models. Models A to D were trained with different input variables; A: SoC clinical risk model, including Age and PSA, B: Methylation model, C: ExoRNA model and D: ExoMeth model, combining the predictors from all three previous models. The full list of variables in each model is available in Table 1. Fill colour shows the risk score distribution of patients with with respect to biopsy outcome: No evidence of cancer (Blue), Gleason 6 or 3+4 (Orange), Gleason ≥4+3 (Green).

FIG. 11—Cumming estimation plot of the ExoMeth risk signatures in No evidence of cancer (NEC) and raised PSA, negative biopsy samples. The left panel details individual patients as points with ExoMeth risk score on the y-axis. Points are coloured according to clinical risk category; NEC—No evidence of cancer, Raised PSA—Raised PSA with negative biopsy. The right panel shows the mean differences in risk score between each NEC and Raised PSA samples. Mean differences and 95% confidence interval are displayed as a point estimate and vertical bar respectively, using the sample density distributions calculated from a bias-corrected and accelerated bootstrap analysis from 1,000 resamples.

FIG. 12—Boruta analysis of variables available for the training of the ExoGrail model. Variable importance was determined over 1,000 bootstrap resamples of the available data and the decision reached recorded at each resample. Colour indicates the proportion of the 1,000 resamples a variable was confirmed to be important in. Variables confirmed in at least 90% of resamples were selected for predictive modelling (Green). Those variables rejected in every single resample are not shown here, but the full list of inputs for all models can be seen in Table 1.

FIG. 13—Waterfall plot of the ExoGrail risk score for each patient. Each coloured bar represents an individual patient's calculated risk score and their true biopsy outcome, coloured according to Gleason score (Gs). Green—No evidence of cancer, Blue—Gs 6, Orange—Gs 3+4, Red—Gs≥ 4+3.

FIG. 14—Density plots detailing risk score distributions generated from fourtrained models. Models A to D were trained with different input variables; A—SoC clinical risk model, including Age and PSA, B—EN2 model, C-ExoRNA model and D—ExoGrail model, combining predictors from all three modes of analysis. The full list of variables in each model is available in Table 1. Fill colour shows the risk score distribution of patients with with respect to biopsy outcome: No evidence of cancer (Green), Gleason 6 (Blue), Gleason 3+4 (Orange), Gleason ≥4+3 (Red). AUCs of each model's predictive ability for clinically relavent biopsy outcomes are detailed underneath each plot.

FIG. 15—Cumming estimation plot of the ExoGrail risk signature. The top row details individual patients as points, separated according to Gleason score on the x-axis and risk score on the y-axis. Points are coloured according to clinical risk category; NEC—No evidence of cancer, Raised PSA—Raised PSA with negative biopsy, L-D'Amico Low-Risk, I—D'Amico Intermediate Risk, H—D'Amico High-Risk. Gapped vertical lines detail the mean and standard deviation of each group's risk scores. The lower panel shows the mean differences in risk score of each group, as compared to the NEC samples. Mean differences and 95% confidence interval are displayed as a point estimate and vertical bar respectively, using the sample density distributions calculated from a bias-corrected and accelerated bootstrap analysis from 1,000 resamples.

FIG. 16—Decision curve analysis (DCA) plots detailing the standardised net benefit (sNB) of adopting different risk models for aiding the decision to biopsy patients who present with a PSA≥4 ng/mL. The x-axis details the range of risk a clinician or patient may accept before deciding to biopsy. Panels show the sNB based upon the detection of varying levels of disease severity: A—detection of Gleason ≥4+3, B—detection of Gleason ≥3+4, C—any cancer; Blue—biopsy all patients with a PSA≥4 ng/mL, Orange—biopsy patients according to the SOC model, Green—biopsy patients based on the methylation model, Purple—biopsy patients based on the ExoRNA model, Red—biopsy patients based on a the ExoGrail model. To assess the benefit of adopting these risk models in a non-PSA screened population we used data available from the control arm of the CAP study [13]. DCA curves were calculated from 1,000 bootstrap resamples of the available data to match the distribution of disease reported in the CAP trial population. Mean sNB from these resampled DCA results are plotted here.

FIG. 17—Net percentage reduction in biopsies, as calculated by DCA measuring the benefit of adopting different risk models for aiding the decision to biopsy patients who would otherwise undergo biopsy by current clinical guidelines. The x-axis details the range of accepted risk a clinician or patient may accept before deciding to biopsy. Panels show the reduction in biopsies per 100 patients based upon the detection of varying levels of disease severity: A—detection of Gleason ≥4+3, B—detection of Gleason ≥3+4 and C—any cancer. Coloured lines show differing comparator models; Blue—biopsy all patients with a PSA>3 ng/mL, Orange—biopsy patients by according the to the SoC model, Green—biopsy patients based on the methylation model, Purple—biopsy patients based on the ExoRNA model, Red—biopsy patients based on a the ExoGrail model. To assess the benefit of adopting these risk models in a non-PSA screened population we used data available from the control arm of the CAP study [13]. DCA curves were calculated from 1,000 bootstrap resamples of the available data to match the distribution of disease reported in the CAP trial population. Mean sNB from these resampled DCA results are used to calculate the potentially reductions in biopsy rates here.

FIG. 18—Boruta analysis of variables available for the training of the SoC model. Variable importance was determined over 1,000 bootstrap resamples of the available data and the decision reached recorded at each resample. Variable origins are denoted by font; clinical variables are italicised and emboldened. Colour indicates the proportion of the 1,000 resamples a variable was confirmed to be important in. Variables confirmed in at least 90% of resamples were selected for training predictive models (Green).

FIG. 19—Boruta analysis of variables available for the training of the Methylation model. Variable importance was determined over 1,000 bootstrap resamples of the available data and the decision reached recorded at each resample. Variable origins are denoted by font; methylation variables are italicised. Colour indicates the proportion of the 1,000 resamples a variable was confirmed to be important in. Variables confirmed in at least 90% of resamples were selected for training predictive models (Green).

FIG. 20—Boruta analysis of variables available for the training of the ExoRNA model (ExoGrail comparator). Variable importance was determined over 1,000 bootstrap resamples of the available data and the decision reached recorded at each resample. Variable origins are denoted by font; clinical variables are emboldened. Colour indicates the proportion of the 1,000 resamples a variable was confirmed to be important in. Variables confirmed in at least 90% of resamples were selected for training predictive models. Those variables rejected in every single resample are not shown here, but the full list of inputs for the ExoRNA model can be seen in Table 1.

FIG. 21—Partial dependency plots detailing the marginal effects and interactions of SLC12A1 and urinary EN2 on predicted ExoGrail Risk Score. A—Partial dependency of ExoGrail on urinary EN2, B—Partial dependency of ExoGrail on SLC12A1, C—Partial dependency of ExoGrail on both SLC12A1 and urinary EN2.

FIG. 22—Density plots detailing risk score distributions generated from fourtrained models. Models A to D were trained with different input variables; A—SoC clinical risk model, including Age and PSA, B—Methylation model, C-ExoRNA model and D—ExoGrail model, combining the predictors from all three previous models. The full list of variables in each model is available in Table 1. Fill colour shows the risk score distribution of patients with a significant biopsy outcome of Gs≥3+4 (Orange) or Gs≤6 (Blue)

FIG. 23—Cumming estimation plot of the ExoGrail risk signatures in No evidence of cancer (NEC) and raised PSA, negative biopsy samples. The left panel details individual patients as points with ExoGrail risk score on the y-axis. Points are coloured according to clinical risk category; NEC—No evidence of cancer, Raised PSA—Raised PSA with negative biopsy. The right panel shows the mean differences in risk score between each NEC and Raised PSA samples. Mean differences and 95% confidence interval are displayed as a point estimate and vertical bar respectively, using the sample density distributions calculated from a bias-corrected and accelerated bootstrap analysis from 1,000 resamples.

FIG. 24—Example computer apparatus.

### DETAILED DESCRIPTION OF THE INVENTION

### Extracellular Vesicles

It is well documented that eukaryotic cells release extracellular vesicles including apoptotic bodies, exosomes, and other microvesicles [34,35]. Here we will use the term Extracellular Vesicle (EV) to include any membranous vesicles found in the urine such as exosomes. Extracellular vesicles differ in their cellular origins and sizes, for example, apoptotic bodies are released from the cell membrane as the final consequence of cell fragmentation during apoptosis, and they have irregular shapes with a range of 1-5 μm in size [35].

Exosomes are specialised vesicles, 30 to 100 nm in size that are actively secreted by a variety of normal and tumour cells and are present in many biological fluids, including serum and urine. They carry membrane and cytosolic components including protein and RNA into the extracellular space [36,37]. These microvesicles form as a result of inward budding of the cellular endosomal membrane resulting in the accumulation of intraluminal vesicles within large multivesicular bodies. Through this process trans-membrane proteins are incorporated into the invaginating membrane while the cytosolic components are engulfed within the intraluminal vesicles that form the exosomes, which will then be released, into the extracellular space [38, 39].

So far urine exosomes have been examined in several studies for renal and prostatic pathology and have been reported to be stable in urine. RNA isolated from urine EVs had a better-preserved profile than cell-isolated RNA from the same samples [40] which makes them much better for potential biomarker use.

### EV Function

EVs such as exosomes function as a means of transport for biological material between cells within an organism. As a consequence of their origin, EVs such as exosomes exhibit the mother-cell's membrane and cytoplasmic components such as proteins, lipids and genomic materials. Some of the proteins they exhibit regulate their docking and membrane fusion, for example the Rab proteins, which are the largest family of small GTPases [41]. Annexins and flotillin aid in membrane-trafficking and fusion events [42]. Exosomes also contain proteins that have been termed exosomal-marker-proteins, for example Alix, TSG101, HSP70 and the tetraspanins CD63, CD81 and CD9. Exosome protein composition is very dependent on the cell type of origin. So far a total of 13,333 exosomal proteins have been reported in the ExoCarta database, mainly from dendritic, normal and malignant cells.

Besides proteins, 2,375 mRNAs and 764 microRNAs have been reported (Exocarta.org) which can be delivered to recipient cells. Exosomes are rich in lipids such as cholesterol, sphingolipids, ceramide and glycerophospolipids which play an important role in exosome biogenesis, especially ILV formation.

### EVs in Malignancy

The role of EVs such as EVs in cancer remains to be fully elucidated; they appear to function as both pro- and anti-tumour effectors. Either way cancer cell-derived EVs appear to have distinct biologic roles and molecular profiles. They can have unique gene expression signatures (RNAs, mRNAs) and proteomics profiles compared to EVs from normal cells [43,44]. Reference 43 reports large numbers of differentially expressed RNAs in EVs from melanocytes compared with melanoma-derived EVs. This indicates that exosomal RNAs may contribute to important biological functions in normal cells, as well as promoting malignancy in tumour cells. Reference 43 also suggests that cancer cell-derived EVs have a closer relationship to the originating cancer cell than normal cell derived EVs do to a normal cell, which highlights the potential of using EVs as a source of diagnostic biomarkers. RNA expression in melanoma EVs has been linked to the advancement of the disease supporting the idea that EVs such as exosomes can promote tumour growth. A similar finding was reported in glioblastoma, highlighting their potential as prognostic markers.

Experiments in mice have shown that cancer-derived EVs can induce an anti-tumour immune response. It has been demonstrated that EVs such as exosomes isolated from malignant effusions are an effective source of tumour antigens which are used by the host to present to CD8+ cytotoxic T cells, dramatically increasing the anti-tumour immune response.

### EVs and Prostate Cancer

Several studies have examined the role of EVs such as exosomes in prostate cancer. Reference 45 suggests that prostate cancer derived EVs can stimulate fibroblast activation and lead to cancer development by increasing cell motility and preventing cell apoptosis. Similarly, vesicles from activated fibroblasts are, in turn, able to induce migration and invasion in the PC3 cell line. Another study reported that EVs from hormone refractory PC cells are able to induce osteoblast differentiation via the Ets1 which they contained, suggesting a role for vesicles in cell-to-cell communication during the osteoblastic metastasis process. Cell-to-cell communication was also emphasised in another study that showed that vesicles released from the human prostate carcinoma cell line DU145 are able to induce transformation in a non-malignant human prostate epithelial cell line.

Besides the in vivo evidence on the active role of EVs in cancer and cancer metastasis, Reference 46 suggests that EVs are present in high levels in the urine of cancer patients, and that unlike cells, EVs have remarkable stability in urine [47]. Other studies suggest the presence of EVs in prostatic secretions, identifying them as a potential source of prostate cancer biomarkers.

Using a nested PCR-based approach, the authors of reference 48 suggest that tumour EVs are harvestable from urine samples from PC patients and that they carry biomarkers specific to PC including KLK3, PCA3 and TMPRSS2/ERG RNAs. PCA3 transcripts were detectable in all patients including subjects with low grade disease, however TMPRSS2/ERG transcripts were only detectable in high Gleason grades. They also demonstrated in this study that i) mild prostate massage increased the extracellular vesicle secretion into the urethra and subsequently into the collected urine fraction ii) that tumour EVs are distinct from EVs shed by normal cells, and iii) they are more abundant in cancer patients.

In the present invention the RNA may be harvested from all extracellular vesicles (EV) present in urine that are below 0.8 μm. The EVs will consist of exosomes and other extracellular vesicles. In further embodiments of the invention different subtypes of EVs may be harvested and analysed.

In some embodiments of the invention RNA is extracted from urine supernatant. In some embodiments of the invention RNA is extracted from whole urine.

### Apparatus and Media

The present invention also provides an apparatus configured to perform any method of the invention.

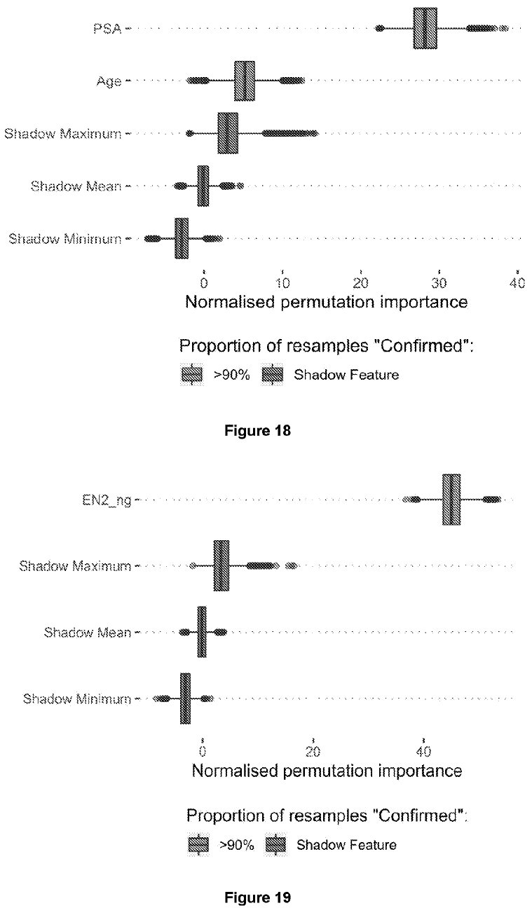

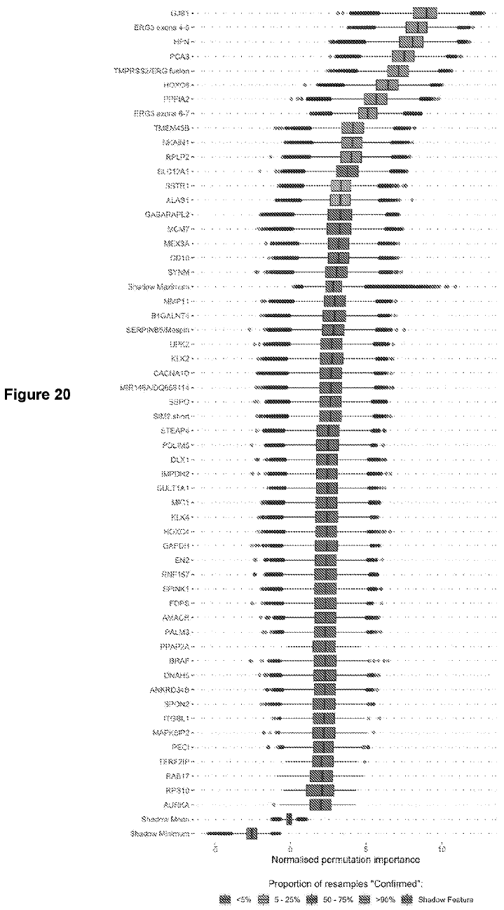

FIG. 18 shows an apparatus or computing device 100 for carrying out a method as disclosed herein. Other architectures to that shown in FIG. 18 may be used as will be appreciated by the skilled person.

Referring to the Figure, the meter 100 includes a number of user interfaces including a visual display 110 and a virtual or dedicated user input device 112. The meter 100 further includes a processor 114, a memory 116 and a power system 118. The meter 100 further comprises a communications module 120 for sending and receiving communications between processor 114 and remote systems. The meter 100 further comprises a receiving device or port 122 for receiving, for example, a memory disk or non-transitory computer readable medium carrying instructions which, when operated, will lead the processor 114 to perform a method as described herein.

The processor 114 is configured to receive data, access the memory 116, and to act upon instructions received either from said memory 116, from communications module 120 or from user input device 112. The processor controls the display 110 and may communicate date to remote parties via communications module 120.

The memory 116 may comprise computer-readable instructions which, when read by the processor, are configured to cause the processor to perform a method as described herein.

The present invention further provides a machine-readable medium (which may be transitory or non-transitory) having instructions stored thereon, the instructions being configured such that when read by a machine, the instructions cause a method as disclosed herein to be carried out.

### Active Surveillance

Active surveillance (AS) is a means of disease-management for men with localised PCa with the intent to intervene if the disease progresses. AS is offered as an option to men whose prostate cancer is thought to have a low risk of causing harm in the absence of treatment. It is a chance to delay or avoid aggressive treatment such as radiotherapy or surgery, and the associated morbidities of these treatments. Entry criteria for men to go on active surveillance varies widely and can include men with Low risk and Intermediate risk prostate cancer.

Patients on AS are currently monitored by a wide range of means that include, for example, PSA monitoring, biopsy and repeat biopsy and MP-MRI. The timing of repeat biopsies, PSA testing and MP-MRI varies with the hospital, and a widely accepted method for monitoring men on AS has not yet been achieved.

In some embodiments, active surveillance comprises assessment of a patient by PSA monitoring, biopsy and repeat biopsy and/or imaging techniques such as MRI, for example MP-MRI. In some embodiments, active surveillance comprises assessment of a patient by any means appropriate for diagnosing or prognosing prostate cancer.

In some embodiments of the invention, active surveillance comprises assessment of a patient at least every 1 month, 2 months, 3 months, 4 months, 5 months, 6 months, 7 months, 8 months, 9 months, 10 months, 11 months or 12 months.

In some embodiments of the invention, active surveillance comprises assessment of a patient at least every 1 year, 2 years, 3 years, 4 years or 5 or more years.

In some embodiments of the invention the ExoMeth and/or ExoGrail risk score will be used alone or in conjunction with other means of testing to improve shared decision making with the multi-disciplinary team and the patient. The ExoMeth and/or ExoGrail risk score could be used to decide whether radical intervention is necessary, or to decide the optimal time between re-monitoring by, for example, biopsy, PSA testing or MP-MRI.

### Biological Samples

In the present invention, the biological sample may be a urine sample, a semen sample, a prostatic exudate sample, or any sample containing macromolecules or cells originating in the prostate, a whole blood sample, a serum sample, saliva, or a biopsy (such as a prostate tissue sample or a tumour sample), although urine samples are particularly useful. The method may include a step of obtaining or providing the biological sample, or alternatively the sample may have already been obtained from a patient, for example in ex vivo methods.

Biological samples obtained from a patient can be stored until needed. Suitable storage methods include freezing immediately, within 2 hours or up to two weeks after sample collection. Maintenance at −80° C. can be used for long-term storage. Preservative may be added, or the urine collected in a tube containing preservative. Urine plus preservative such as Norgen urine preservative, can be stored between room temperature and −80° C.

Methods of the invention may comprise steps carried out on biological samples. The biological sample that is analysed may be a urine sample, a semen sample, a prostatic exudate sample, or any sample containing macromolecules or cells originating in the prostate, a whole blood sample, a serum sample, saliva, or a biopsy (such as a prostate tissue sample or a tumour sample). Most commonly for prostate cancer the biological sample is from a prostate biopsy, prostatectomy or TURP. The method may include a step of obtaining or providing the biological sample, or alternatively the sample may have already been obtained from a patient, for example in ex vivo methods. The samples are considered to be representative of the expression status of the relevant genes in the potentially cancerous prostate tissue, or other cells within the prostate, or microvesicles produced by cells within the prostate or blood or immune system. Alternatively, the samples can be considered to be representative of the potentially cancerous microenvironment of the prostate, comprising gene expression or methylation and protein expression. Hence the methods of the present invention may use quantitative data on RNA, methylation and proteins produced by cells within the prostate and/or the blood system and/or bone marrow in response to cancer, to determine the presence or absence of prostate cancer.

The methods of the invention may be carried out on one test sample from a patient. Alternatively, a plurality of test samples may be taken from a patient, for example at least 2, 3, 4 or 5 samples. Each sample may be subjected to a separate analysis using a method of the invention, or alternatively multiple samples from a single patient undergoing diagnosis could be included in the method.

The sample may be processed prior to determining the expression status of the biomarkers. The sample may be subject to enrichment (for example to increase the concentration of the biomarkers being quantified), centrifugation or dilution. In other embodiments, the samples do not undergo any pre-processing and are used unprocessed (such as whole urine).

In some embodiments of the invention, the biological sample may be fractionated or enriched for RNA prior to detection and quantification (i.e. measurement). The step of fractionation or enrichment can be any suitable pre-processing method step to increase the concentration of RNA in the sample or select for specific sources of RNA such as cells or extracellular vesicles. For example, the steps of fractionation and/or enrichment may comprise centrifugation and/or filtration to remove cells or unwanted analytes from the sample, or to increase the concentration of EVs in a urine fraction. Methods of the invention may include a step of amplification to increase the amount of gene transcripts that are detected and quantified. Methods of amplification include RNA amplification, amplification as cDNA, and PCR amplification. Such methods may be used to enrich the sample for any biomarkers of interest.

Generally speaking, the RNAs will need to be extracted from the biological sample. This can be achieved by a number of suitable methods. For example, extraction may involve separating the RNAs from the biological sample. Methods include chemical extraction and solid-phase extraction (for example on silica columns). Preferred methods include the use of a silica column. Methods comprise lysing cells or vesicles (if required), addition of a binding solution, centrifugation in a spin column to force the binding solution through a silica gel membrane, optional washing to remove further impurities, and elution of the nucleic acid. Commercial kits are available for such methods, for example from Qiagen or Exigon.

If RNAs are extracted from a sample, the extracted solution may require enrichment to increase the relative abundance of RNA transcripts in the sample.

The methods of the invention may be carried out on one test sample from a patient. Alternatively, a plurality of test samples may be taken from a patient, for example at least 2, at least 3, at least 4 or at least 5 samples.

Each sample may be subjected to a single assay to quantify one of the biomarker panel members, or alternatively a sample may be tested for all of the biomarkers being quantified.

### Methods of the Invention

### Expression Status

Determining the expression status of a gene may comprise determining the level of expression of the gene. Expression status also encompasses the determination of any parameter of a gene or protein which impacts the functional effect of the gene or protein in question. For example, this encompasses, among other parameters, the methylation status, the level of mRNA (i.e. gene transcripts) and/or the concentration of protein. Expression status and levels of expression as used herein can be determined by methods known to the skilled person. For example, this may refer to the up or down-regulation of a particular gene or genes, as determined by methods known to a skilled person. Epigenetic modifications may be used as an indicator of expression, for example determining DNA methylation status, or other epigenetic changes such as histone marking, RNA changes or conformation changes. Epigenetic modifications regulate expression of genes in DNA and can influence efficacy of medical treatments among patients. Aberrant epigenetic changes are associated with many diseases such as, for example, cancer. DNA methylation in animals influences dosage compensation, imprinting, and genome stability and development. Methods of determining DNA methylation are known to the skilled person (for example methylation-specific PCR, matrix-assisted laser desorption/ionization time-of-flight mass spectrometry, use of microarrays, reduced representation bisulfate sequencing (RRBS) or whole genome shotgun bisulfate sequencing (WGBS). In addition, epigenetic changes may include changes in conformation of chromatin. The impact of different parameters (for example methylation status) is known to the skilled person. In many cases, the impact of the altered parameter will be clear, for example higher protein concentration leading to a greater availability of the protein to achieve its effect.

### Expression Analysis

NanoString® technology is based on double hybridisation of two adjacent ˜50 bp probes to their target RNA/cDNA. The first probe hybridisation is used to pull the target RNA/cDNA down on to a hard surface. The excess unbound nucleic acid is then washed away. The second probe is then hybridised to the RNA/cDNA. This probe has a multi-colour barcode attached to it. The nucleotides are then stretched out under an electrical current, and the image is recorded. The barcodes number and type are counted, and this is the data output. Up to 800 different barcodes are possible, and therefore up to 800 different target RNAs can be detected in a single assay.

Methods of real-time qPCR may involve a step of reverse transcription of RNA into complementary DNA (cDNA). PCR amplification can use sequence specific primers or combinations of other primers to amplify RNA species of interest. Microarray analysis may comprise the steps of labelling RNA or cDNA, hybridisation of the labelled RNAs to DNA (or RNA or LNA) probes on a solid-substrate array, washing the array, and scanning the array.

RNA sequencing is another method that can benefit from RNA enrichment, although this is not always necessary. RNA sequencing techniques generally use next generation sequencing methods (also known as high-throughput or massively parallel sequencing). These methods use a sequencing-by-synthesis approach and allow relative quantification and precise identification of RNA sequences. In situ hybridisation techniques can be used on tissue samples, both in vivo and ex vivo.

In some methods of the invention, detection and quantification of cDNA-binding molecule complexes may be used to determine RNA expression. For example, RNA transcripts in a sample may be converted to cDNA by reverse-transcription, after which the sample is contacted with binding molecules specific for the RNAs being quantified, detecting the presence of a of cDNA-specific binding molecule complex, and quantifying the expression of the corresponding gene. There is therefore provided the use of cDNA transcripts corresponding to one or more of the RNAs of interest, or combinations thereof, for use in methods of detecting, diagnosing or predicting prognosis of prostate. In some embodiments of the invention, the method may therefore comprise a step of conversion of the RNAs to cDNA to allow a particular analysis to be undertaken and to achieve RNA quantification.

DNA and RNA arrays (microarrays) for use in quantification of the mRNAs of interest comprise a series of microscopic spots of DNA or RNA sequences, each with a unique sequence of nucleotides that are able to bind complementary nucleic acid molecules. In this way the oligonucleotides are used as probes to which only the correct target sequence will hybridise under high-stringency condition. In the present invention, the target sequence can be the coding DNA sequence or unique section thereof, corresponding to the RNA whose expression is being detected. Most commonly the target sequence is the RNA biomarker of interest itself.

Capture molecules include antibodies, proteins, aptamers, nucleic acids, biotin, streptavidin, receptors and enzymes, which might be preferable if commercial antibodies are not available for the analyte being detected. Capture molecules for use on the arrays can be externally synthesised, purified and attached to the array. Alternatively, they can be synthesised in-situ and be directly attached to the array. The capture molecules can be synthesised through biosynthesis, cell-free DNA expression or chemical synthesis. In-situ synthesis is possible with the latter two. The appropriate capture molecule will depend on the nature of the target (e.g. RNA, protein or cDNA).

Once captured on a microarray, detection methods can be any of those known in the art. For example, fluorescence detection can be employed. It is safe, sensitive and can have a high resolution. Other detection methods include other optical methods (for example colorimetric analysis, chemiluminescence, label free Surface Plasmon Resonance analysis, microscopy, reflectance etc.), mass spectrometry, electrochemical methods (for example voltammetry and amperometry methods) and radio frequency methods (for example multipolar resonance spectroscopy).

Once the expression status or concentration has been determined, the level can be compared to a threshold level or previously measured expression status or concentration (either in a sample from the same subject but obtained at a different point in time, or in a sample from a different subject, for example a healthy subject, i.e. a control or reference sample) to determine whether the expression status or concentration is higher or lower in the sample being analysed. Hence, the methods of the invention may further comprise a step of correlating said detection or quantification with a control or reference to determine if prostate cancer is present (or suspected) or not. Said correlation step may also detect the presence of a particular type, stage, grade or risk group of prostate cancer and to distinguish these patients from healthy patients, in which no prostate cancer is present or from men with indolent or low risk disease. For example, the methods may detect early stage or low risk prostate cancer. Said step of correlation may include comparing the amount (expression or concentration) of one, two, or three or more of the panel biomarkers with the amount of the corresponding biomarker(s) in a reference sample, for example in a biological sample taken from a healthy patient. The methods of the invention may include the steps of determining the amount of the corresponding biomarker in one or more reference samples which may have been previously determined. Alternatively, the method may use reference data obtained from samples from the same patient at a previous point in time. In this way, the effectiveness of any treatment can be assessed and a prognosis for the patient determined.

Internal controls can be also used, for example quantification of one or more different RNAs not part of the biomarker panel. This may provide useful information regarding the relative amounts of the biomarkers in the sample, allowing the results to be adjusted for any variances according to different populations or changes introduced according to the method of sample collection, processing or storage.

Methods of normalisation can involve correction of the counts of the measured levels of NanoString® gene-probes in order to account for, for example; differences in the input amount of RNA, variability in RNA quality and to centre data around RNA originating from prostatic material, so that all the genes being analysed are on a comparable scale.

As would be apparent to a person of skill in the art, any measurements of analyte concentration or expression may need to be normalised to take in account the type of test sample being used and/or and processing of the test sample that has occurred prior to analysis. Data normalisation also assists in identifying biologically relevant results. Invariant RNAs/mRNAs may be used to determine appropriate processing of the sample. Differential expression calculations may also be conducted between different samples to determine statistical significance.

### Further Analytical Methods Used in the Invention

The expression status of a gene or protein from a biomarker panel of the invention can be determined in a number of ways. Levels of expression may be determined by, for example, quantifying the biomarkers by determining the concentration of protein in the sample, if the biomarkers are expressed as a protein in that sample. Alternatively, the amount of RNA or protein in the sample (such as a tissue sample) may be determined. Once the expression status has been determined, the level can optionally be compared to a control. This may be a previously measured expression status (either in a sample from the same subject but obtained at a different point in time, or in a sample from a different subject or subjects, for example one or more healthy subjects or one or more subjects with non-aggressive cancer, i.e. a control or reference sample) or to a different protein or peptide or other marker or means of assessment within the same sample to determine whether the expression status or protein concentration is higher or lower in the sample being analysed. Housekeeping genes can also be used as a control. Ideally, controls are one or more RNA, protein or DNA markers that generally do not vary significantly between samples or between tissue from different people or between normal tissue and tumour.

Other methods of quantifying gene expression include RNA sequencing, which in one aspect is also known as whole transcriptome shotgun sequencing (WTSS). Using RNA sequencing it is possible to determine the nature of the RNA sequences present in a sample, and furthermore to quantify gene expression by measuring the abundance of each RNA molecule (for example, RNA or microRNA transcripts). The methods use sequencing-by-synthesis approaches to enable high throughout analysis of samples.

There are several types of RNA sequencing that can be used, including RNA PolyA tail sequencing (there the polyA tail of the RNA sequences are targeting using polyT oligonucleotides), random-primed sequencing (using a random oligonucleotide primer), targeted sequence (using specific oligonucleotide primers complementary to specific gene transcripts), small RNA/non-coding RNA sequencing (which may involve isolating small non-coding RNAs, such as microRNAs, using size separation), direct RNA sequencing, and real-time PCR. In some embodiments, RNA sequence reads can be aligned to a reference genome and the number of reads for each sequence quantified to determine gene expression. In some embodiments of the invention, the methods comprise transcription assembly (de-novo or genome-guided).

RNA, DNA and protein arrays (microarrays) may be used in certain embodiments. RNA and DNA microarrays comprise a series of microscopic spots of DNA or RNA oligonucleotides, each with a unique sequence of nucleotides that are able to bind complementary nucleic acid molecules. In this way the oligonucleotides are used as probes to which the correct target sequence will hybridise under high-stringency condition. In the present invention, the target sequence can be the transcribed RNA sequence or unique section thereof, corresponding to the gene whose expression is being detected. Protein microarrays can also be used to directly detect protein expression. These are similar to DNA and RNA microarrays in that they comprise capture molecules fixed to a solid surface.

Methods for detection of RNA or cDNA can be based on hybridisation, for example, Northern blot, Microarrays, NanoString®, RNA-FISH, branched chain hybridisation assay, or amplification detection methods for quantitative reverse transcription polymerase chain reaction (qRT-PCR) such as TaqMan, or SYBR green product detection. Primer extension methods of detection such as: single nucleotide extension, Sanger sequencing. Alternatively, RNA can be sequenced by methods that include Sanger sequencing, Next Generation (high throughput) sequencing, in particular sequencing by synthesis, targeted RNAseq such as the Precise targeted RNAseq assays, or a molecular sensing device such as the Oxford Nanopore MinION device. Combinations of the above techniques may be utilised such as Transcription Mediated Amplification (TMA) as used in the Gen-Probe PCA3 assay which uses molecule capture via magnetic beads, transcription amplification, and hybridisation with a secondary probe for detection by, for example chemiluminescence.

RNA may be converted into cDNA prior to detection. RNA or cDNA may be amplified prior or as part of the detection.

The test may also constitute a functional test whereby presence of RNA or protein or other macromolecule can be detected by phenotypic change or changes within test cells. The phenotypic change or changes may include alterations in motility or invasion.

Commonly, proteins subjected to electrophoresis are also further characterised by mass spectrometry methods. Such mass spectrometry methods can include matrix-assisted laser desorption/ionisation time-of-flight (MALDI-TOF).

MALDI-TOF is an ionisation technique that allows the analysis of biomolecules (such as proteins, peptides and sugars), which tend to be fragile and fragment when ionised by more conventional ionisation methods. Ionisation is triggered by a laser beam (for example, a nitrogen laser) and a matrix is used to protect the biomolecule from being destroyed by direct laser beam exposure and to facilitate vaporisation and ionisation. The sample is mixed with the matrix molecule in solution and small amounts of the mixture are deposited on a surface and allowed to dry. The sample and matrix co-crystallise as the solvent evaporates.

Additional methods of determining protein concentration include mass spectrometry and/or liquid chromatography, such as LC-MS, UPLC, a tandem UPLC-MS/MS system, and ELISA methods. Other methods that may be used in the invention include Agilent bait capture and PCR-based methods (for example PCR amplification may be used to increase the amount of analyte).

Methods of the invention can be carried out using binding molecules or reagents specific for the analytes (RNA molecules or proteins being quantified). Binding molecules and reagents are those molecules that have an affinity for the RNA molecules or proteins being detected such that they can form binding molecule/reagent-analyte complexes that can be detected using any method known in the art. The binding molecule of the invention can be an oligonucleotide, or oligoribonucleotide or locked nucleic acid or other similar molecule, an antibody, an antibody fragment, a protein, an aptamer or molecularly imprinted polymeric structure, or other molecule that can bind to DNA or RNA. Methods of the invention may comprise contacting the biological sample with an appropriate binding molecule or molecules. Said binding molecules may form part of a kit of the invention, in particular they may form part of the biosensors of in the present invention.

Aptamers are oligonucleotides or peptide molecules that bind a specific target molecule. Oligonucleotide aptamers include DNA aptamer and RNA aptamers. Aptamers can be created by an in vitro selection process from pools of random sequence oligonucleotides or peptides. Aptamers can be optionally combined with ribozymes to self-cleave in the presence of their target molecule. Other oligonucleotides may include RNA molecules that are complimentary to the RNA molecules being quantified. For example, polyT oligos can be used to target the polyA tail of RNA molecules.

Aptamers can be made by any process known in the art. For example, a process through which aptamers may be identified is systematic evolution of ligands by exponential enrichment (SELEX). This involves repetitively reducing the complexity of a library of molecules by partitioning on the basis of selective binding to the target molecule, followed by re-amplification. A library of potential aptamers is incubated with the target protein before the unbound members are partitioned from the bound members. The bound members are recovered and amplified (for example, by polymerase chain reaction) in order to produce a library of reduced complexity (an enriched pool). The enriched pool is used to initiate a second cycle of SELEX. The binding of subsequent enriched pools to the target protein is monitored cycle by cycle. An enriched pool is cloned once it is judged that the proportion of binding molecules has risen to an adequate level. The binding molecules are then analysed individually. SELEX is reviewed in [49].

### Statistical Analysis

### Decision Curve Analysis (DCA)

Decision curve analysis is a method of evaluating predictive models. It assumes that the threshold probability of a disease or event at which a patient would opt for treatment is informative of how the patient weighs the relative harms of a false-positive and a false-negative prediction. This theoretical relationship is then used to derive the net benefit of the model across different threshold probabilities. Plotting net benefit against threshold probability yields the “decision curve.” Decision curve analysis can be used to identify the range of threshold probabilities in which a model is of value, the magnitude of benefit, and which of several models is optimal [50].

### Boruta Algorithm

The Boruta algorithm is a wrapper built around the random forest classification algorithm. It duplicates a dataset, and randomly shuffles the values in each column. These values are called shadow features. It then trains a classifier, such as a Random Forest Classifier, on the dataset. By doing this, it can provide an idea of the importance—via the Mean Decrease Accuracy or Mean Decrease Impurity—for each of the features of the data set. The higher the score, the better or more important the feature is.

The algorithm checks whether each of the “real” features have higher importance than the “shadow” features. In other words, whether the feature has a higher Z-score (i.e. the number of standard deviations from the mean a data point is) than the maximum Z-score of the best of the shadow features. If the algorithm identifies a “real” feature with a better association than the “shadow” features then it will record this as a hit. After a predefined set of iterations, the algorithm provides a table of hits.

At every iteration (or resample), the algorithm compares the Z-scores of the shuffled copies of the features and the original features to see if the latter performed better than the former. If it does, the algorithm will mark the feature as important. In essence, the algorithm validates the importance of the feature by comparing with random shuffled copies, which increases the robustness. This is done by simply comparing the number of times a feature did better with the shadow features using a binomial distribution.

The number of iterations can be predefined. In some aspects of the invention the number of iterations (or resamples) is at least about 100, about 200, about 300, about 400, about 500, about 600, about 700, about 800, about 900, about 1000, about 1500, about 2000, about 3000, about 4000, about 5000. In a preferred embodiment of the invention the number of iterations (or resamples) is 1000.

The proportion of iterations (or resamples) in which a feature must be selected in order to be considered associated with a biopsy outcome group can be predefined. In some aspects of the invention the proportion of iterations (or resamples) in which a feature must be selected in order to be considered associated with a biopsy outcome group is at least about 70%, about 75%, about 80%, about 85%, about 90%, about 95%, about 98% or about 99%. In a preferred embodiment of the invention the proportion of iterations (or resamples) in which a feature must be selected in order to be considered associated with a biopsy outcome group is 90%.

### Gene Transcript Detection

The present invention provides probes suitable for use in cDNA or RNA sequence detection such as NanoString® or microarray techniques which can be used to determine the expression status of genes of interest. Methods of the invention can be operated using any suitable probe sequence to detect a gene transcript and methods of generating probe sequences are known to those skilled in the art.

In another embodiment the gene transcripts may be detected by sequencing, or qRT-PCR.

### Methylation Detection

The methylation status of genes can be determined by any suitable means. For example, methylation detection assays which rely on the digestion of genomic DNA with a methylation-sensitive restriction enzyme followed by either Southern blot analysis or PCR. Other suitable assays use treatment of genomic DNA with sodium bisulfite followed by alkaline treatment to convert unmethylated cytosines to uracil, while leaving methylated cytosine residues intact. Sequence variants at a particular locus can subsequently be analyzed by PCR amplification with primers designed to anneal with bisulfite-converted DNA. Preferably, methylation status of genes is established using high-throughput assays that utilize highly sensitive and accurate fluorescence-based real-time quantitative PCR (qPCR). Other suitable methods will be known in the art.

### Protein Quantification

The concentration of a urinary protein can be established by any suitable method. Individual protein quantitation methods include enzyme-linked immunosorbent assay (ELISA) assay, western blot analysis, and more recently, mass spectrometry, among others. ELISAs are used to qualitatively and quantitatively analyze the presence or concentration of a particular soluble antigen, peptide or protein in liquid samples, such as biological fluids. These assays make use of the ability of polystyrene plates to bind proteins, including antibodies, as well as the particular specificities of antibodies for target antigens. Generally, these assays incorporate a colorimetric endpoint that can be detected via absorbance wavelength and quantitated from a known standard curve of antigen or antibody dilutions. Western blotting is a method in which proteins that have been electrophoretically separated on a gel are transferred to an absorbent membrane via an electric charge. Once blotted, the proteins can be detected with labeled specific antibodies. Preferably the concentration of protein is detected by ELISA assay. Other suitable methods will be known in the art.

### Biopsies

A prostate biopsy involves taking a sample of the prostate tissue, for example by using thin needles to take small samples of tissue from the prostate. The tissue is then examined under a microscope to check for cancer.

There are two main types of prostate biopsy—a TRUS (trans-rectal ultrasound) guided or transrectal biopsy, and a template (transperineal) biopsy. TRUS biopsy involves insertion of an ultrasound probe into the rectum and scanning the prostate in order to guide where to extract the cells from. Normally 10 to 12 small pieces of tissue are taken from different areas of the prostate.

A template biopsy involves inserting the biopsy needle into the prostate through the skin between the testicles and the rectum (the perineum). The needle is inserted through a grid (template). A template biopsy takes more tissue samples from more areas of the prostate than a TRUS biopsy. The number of samples taken will vary but can be around 20 to 50 from different areas of the prostate.

### Prostate Cancer Treatment

Patients with metastatic disease are primarily treated with hormone deprivation therapy. However, the cancer invariably becomes resistant to treatment leading to disease progression and eventually death. Treatment of patients with metastatic prostate cancer is clinically very challenging for a number of reasons, which include: i) the variability in patient response to hormone treatment (i.e. time prior to relapse and becoming castrate resistant), ii) the detrimental effects of hormone manipulation therapy on patients and iii) the myriad new treatment options available for castrate resistant patients. In some cases, treatment of prostate cancer can be placing the patient under active surveillance.

The response to hormone manipulation/ablation therapy is highly variable. Some men fail to respond to treatment while others relapse early (i.e. within 6 months), the majority relapse within 18 months (late relapse) and the rest respond well to the treatment often taking several years before relapsing (delayed relapse). Early identification of patients who will have a poor response will provide a clinical opportunity to offer them a different treatment approach that may perhaps improve their prognosis. However, there is no means currently to identify such patients except for when they exhibit biochemical progression with rising PSA level (e.g. serum PSA level), or become clinically symptomatic, in which case they get offered a different treatment strategy. This regime however goes hand in hand with a number of detrimental effects such as bone loss, increased obesity, decreased insulin sensitivity increasing the incidence of diabetes, adversely altered lipid profiles leading to cardiovascular disease and an increased rate of heart attacks. For these reasons offering hormone manipulation requires a lot of clinical consideration particularly as most of the patients requiring such treatment are elderly patients and such treatment could overall be detrimental rather than beneficial.

Due to ever-emerging new treatments or second line therapies for patients with advanced metastatic cancer in the past decade, the treatment of men with castrate resistant prostate cancer is dramatically changing. Prior to 2004, the only treatment option for these patients was medical or surgical castration then palliation. Since then several chemotherapy treatments have emerged starting with docetaxel, which has shown to improve survival for some patients. This was followed by five additional agents (FDA-approved) including new hormonal agents targeting the androgen receptor (AR) such as the AR antagonist Enzalutamide, agents to inhibit androgen biosynthesis such as Abiraterone, two agents designed specifically to affect the androgen axis, sipuleucel-T, which stimulates the immune system, cabazitaxel chemotherapeutic agent and radium-223, a radionuclide therapy. Other treatments include targeted therapies such as the PI3K inhibitor BKM120 and an Akt inhibitor AZD5363. Therefore, it is crucially important to be able to identify patients that would benefit from these treatments and those that will not. Identification of prognostic indicators capable of predicting response to hormone manipulation and to the above list of alternative treatments is very important and would have great clinical impact in managing these patients. In addition, the only current clinically available means to diagnose metastasis is by imaging. Markers that are being put forward include circulating tumour cells and urine bone degradation markers. A test for metastasis per se could radically alter patient treatment. The data presented here in suggest that extracellular vesicle RNA may have the potential to overcome these issues, particularly as studies have shown a role for EVs such as exosomes in aiding metastasis. A test for metastasis per se could radically alter patient treatment.

Prostate cancers can be staged according to how advanced they are. This is based on the TMN scoring as well as any other factors, such as the Gleason score and/or the PSA test. The staging can be defined as follows:

### Stage I:

T1, N0, M0, Gleason score 6 or less, PSA less than 10

OR

T2a, N0, M0, Gleason score 6 or less, PSA less than 10

### Stage IIA:

T1, N0, M0, Gleason score of 7, PSA less than 20

OR

T1, N0, M0, Gleason score of 6 or less, PSA at least 10 but less than 20:

OR

T2a or T2b, N0, M0, Gleason score of 7 or less, PSA less than 20

### Stage IIB:

T2c, N0, M0, any Gleason score, any PSA

OR

T1 or T2, N0, M0, any Gleason score, PSA of 20 or more:

OR

T1 or T2, N0, M0, Gleason score of 8 or higher, any PSA

### Stage III:

T3, N0, M0, any Gleason score, any PSA

### Stage IV:

T4, N0, M0, any Gleason score, any PSA

OR

Any T, N1, M0, any Gleason score, any PSA:

OR

Any T, any N, M1, any Gleason score, any PSA

In the present invention, an aggressive cancer is defined functionally or clinically: namely a cancer that can progress. This can be measured by PSA failure. When a patient has surgery or radiation therapy, the prostate cells are killed or removed. Since PSA is only made by prostate cells the PSA level in the patient's blood reduces to a very low or undetectable amount. If the cancer starts to recur, the PSA level increases and becomes detectable again. This is referred to as “PSA failure”. An alternative measure is the presence of metastases or death as endpoints.

Prostate cancer can be scored using the Prostate Imaging Reporting and Data System (PI-RADS) grading system designed to standardise non-invasive MRI and related image acquisition and reporting, potentially useful in the initial assessment of the risk of clinically significant prostate cancer. A PI-RADS score is given according to each variable parameter. The scale is based on a score “Yes” or “No” for Dynamic Contrast-Enhanced (DCE) parameter, and from 1 to 5 for T2-weighted (T2W) and Diffusion-weighted imaging (DWI). The score is given for each lesion, with 1 being most probably benign and 5 being highly suspicious of malignancy:

PI-RADS 1: very low (clinically significant cancer is highly unlikely to be present)

PI-RADS 2: low (clinically significant cancer is unlikely to be present)

PI-RADS 3: intermediate (the presence of clinically significant cancer is equivocal)

PI-RADS 4: high (clinically significant cancer is likely to be present)

PI-RADS 5: very high (clinically significant cancer is highly likely to be present)

Increase in Gleason score, stage as defined above or PI-RADS grade can also be considered as progression. However, a ExoMeth and/or ExoGrail risk score is independent of Gleason, stage and PI-RADS. It provides additional information about the development of aggressive cancer in addition to Gleason, stage and PI-RADS. It is therefore a useful independent predictor of outcome. Nevertheless, ExoMeth and/or ExoGrail risk score can be combined with Gleason, tumour stage and/or PI-RADS score.

In some methods of the invention the ExoMeth and/or ExoGrail risk score can be used alongside MRI to aid decision making on whether to biopsy or not, particularly in men with PI-RADS 3 and 4. ExoMeth and/or ExoGrail risk scores could also be used to confirm the absence of clinically significant prostate cancer in men with PI-RADS 1 and 2.

Thus, the methods of the invention provide methods of providing a cancer diagnosis or prognosis based on one or more clinical variables and/or the expression status of a plurality of genes comprising determining the expression status of one or more members of a biomarker panel and/or one or more clinical variables. The expression of one or more members of the panel of markers may be determined using a method of the invention.

By “clinical outcome” it is meant that for each patient whether the cancer has progressed. For example, as part of an initial assessment, those patients may have prostate specific antigen (PSA) levels monitored. When it rises above a specific level, this is indicative of relapse and hence disease progression. Histopathological diagnosis may also be used. Spread to lymph nodes, and metastasis can also be used, as well as death of the patient from the cancer (or simply death of the patient in general) to define the clinical endpoint. Gleason scoring, cancer staging and multiple biopsies (such as those obtained using a coring method involving hollow needles to obtain samples) can be used. Clinical outcomes may also be assessed after treatment for prostate cancer. This is what happens to the patient in the long term. Usually the patient will be treated radically (prostatectomy, radiotherapy) to effectively remove or kill the prostate. The presence of a relapse or a subsequent rise in PSA level (e.g. serum PSA level) (known as PSA failure) is indicative of progressed cancer. The high ExoMeth and/or ExoGrail risk score cancer populations identified using methods of the invention comprise subpopulations of cancers that may progress more quickly.

Accordingly, any of the methods of the invention may be carried out in patients in whom prostate cancer is suspected. Importantly, the present invention allows a prediction of cancer progression before treatment of cancer is provided. This is particularly important for prostate cancer, since many patients will undergo unnecessary treatment for prostate cancer when the cancer would not have progressed even without treatment.

Proteins can also be used to determine expression status, and suitable methods to determine expressed protein levels are known to the skilled person.

The present invention shall now be further described with reference to the following examples, which are present for the purposes of illustration only and are not to be construed as being limiting on the invention.

TABLE 1
List of all features available for selection as input variables for each model used
in the ExoMeth model design prior to bootstrapped Boruta feature selection.

| SoC | Methylation | ExoRNA | ExoMeth | ExoGrail |
|---|---|---|---|---|
| PSA | GSTP1 | March5 | PSA | PSA |
| UrineVol | APC | AATF | UrineVol | UrineVol |
| DRESize | SFRP2 | ABCB9 | DRESize | DRESize |
| Age | IGFBP3 | ACTR5 | Age | Age |
| | IGFBP7 | AGR2 | GSTP1 | Urinary EN2 |
| | PTGS2 | ALAS1 | APC | March5 |
| | | AMACR | SFRP2 | AATF |
| | | AMH | IGFBP3 | ABCB9 |
| | | ANKRD34B | IGFBP7 | ACTR5 |
| | | ANPEP | PTGS2 | AGR2 |
| | | APOC1 | March5 | ALAS1 |
| | | ARexon9 | AATF | AMACR |
| | | ARexons4-8 | ABCB9 | AMH |
| | | ARHGEF25 | ACTR5 | ANKRD34B |
| | | AURKA | AGR2 | ANPEP |
| | | B2M | ALAS1 | APOC1 |
| | | B4GALNT4 | AMACR | ARexon9 |
| | | BRAF | AMH | ARexons4-8 |
| | | BTG2 | ANKRD34B | ARHGEF25 |
| | | CACNA1D | ANPEP | AURKA |
| | | CADPS | APOC1 | B2M |
| | | CAMK2N2 | ARexon9 | B4GALNT4 |
| | | CAMKK2 | ARexons4-8 | BRAF |
| | | CASKIN1 | ARHGEF25 | BTG2 |
| | | CCDC88B | AURKA | CACNA1D |
| | | CD10 | B2M | CADPS |
| | | CDC20 | B4GALNT4 | CAMK2N2 |
| | | CDC37L1 | BRAF | CAMKK2 |
| | | CDKN3 | BTG2 | CASKIN1 |
| | | CKAP2L | CACNA1D | CCDC88B |
| | | CLIC2 | CADPS | CD10 |
| | | CLU | CAMK2N2 | CDC20 |
| | | COL10A1 | CAMKK2 | CDC37L1 |
| | | COL9A2 | CASKIN1 | CDKN3 |
| | | CP | CCDC88B | CKAP2L |
| | | CTA-211A9.5/MIATNB | CD10 | CLIC2 |
| | | DLX1 | CDC20 | CLU |
| | | DNAH5 | CDC37L1 | COL10A1 |
| | | DPP4 | CDKN3 | COL9A2 |
| | | EIF2D | CKAP2L | CP |
| | | EN2 | CLIC2 | CTA-211A9.5/MIATNB |
| | | ERG exons 4-5 | CLU | DLX1 |
| | | ERG exons 6-7 | COL10A1 | DNAH5 |
| | | ERG5 | COL9A2 | DPP4 |
| | | FDPS | CP | EIF2D |
| | | FOLH1/PSMA/NAALAD1 | CTA-211A9.5/MIATNB | EN2 |
| | | GABARAPL2 | DLX1 | ERG exons 4-5 |
| | | GAPDH | DNAH5 | ERG exons 6-7 |
| | | GCNT1 | DPP4 | ERG5 |
| | | GJB1 | EIF2D | FDPS |
| | | GOLM1 | EN2 | FOLH1/PSMA/NAALAD1 |
| | | HIST1H1C | ERG exons 4-5 | GABARAPL2 |
| | | HIST1H1E | ERG exons 6-7 | GAPDH |
| | | HIST1H2BF | ERG5 | GCNT1 |
| | | HIST1H2BG | FDPS | GJB1 |
| | | HIST32HA | FOLH1/PSMA/NAALAD1 | GOLM1 |
| | | HMBS | GABARAPL2 | HIST1H1C |
| | | HOXC4 | GAPDH | HIST1H1E |
| | | HOXC6 | GCNT1 | HIST1H2BF |
| | | HPN | GJB1 | HIST1H2BG |
| | | HPRT | GOLM1 | HIST32HA |
| | | IFT57 | HIST1H1C | HMBS |
| | | IGFBP3 | HIST1H1E | HOXC4 |
| | | IMPDH2 | HIST1H2BF | HOXC6 |
| | | ISX | HIST1H2BG | HPN |
| | | ITGBL1 | HIST32HA | HPRT |
| | | ITPR1 | HMBS | IFT57 |
| | | KLK2 | HOXC4 | IGFBP3 |
| | | KLK3/PSA(exons1-2 | HOXC6 | IMPDH2 |
| | | KLK3/PSA(exons2-3 | HPN | ISX |
| | | KLK4 | HPRT | ITGBL1 |
| | | LASS1 | IFT57 | ITPR1 |
| | | LBH | IGFBP3 | KLK2 |
| | | MAK | IMPDH2 | KLK3/PSA(exons1-2 |
| | | MAPK8IP2 | ISX | KLK3/PSA(exons2-3 |
| | | MCM7 | ITGBL1 | KLK4 |
| | | MCTP1 | ITPR1 | LASS1 |
| | | MDK | KLK2 | LBH |
| | | MED4 | KLK3/PSA(exons1-2 | MAK |
| | | MEMO1 | KLK3/PSA(exons2-3 | MAPK8IP2 |
| | | Met | KLK4 | MCM7 |
| | | MEX3A | LASS1 | MCTP1 |
| | | MFSD2A | LBH | MDK |
| | | MGAT5B | MAK | MED4 |
| | | MIC1 | MAPK8IP2 | MEMO1 |
| | | MIR146A/DQ658414 | MCM7 | Met |
| | | MIR4435-1HG/IOC541471 | MCTP1 | MEX3A |
| | | MKi67 | MDK | MFSD2A |
| | | MMP11 | MED4 | MGAT5B |
| | | MMP25 | MEMO1 | MIC1 |
| | | MMP26 | Met | MIR146A/DQ658414 |
| | | MNX1 | MEX3A | MIR4435-1HG/IOC541471 |
| | | MSMB | MFSD2A | MKi67 |
| | | MX11 | MGAT5B | MMP11 |
| | | MYOF | MIC1 | MMP25 |
| | | NAALADL2 | MIR146A/DQ658414 | MMP26 |
| | | NEAT1 | MIR4435-1HG/IOC541471 | MNX1 |
| | | NKAIN1 | MKi67 | MSMB |
| | | NLRP3 | MMP11 | MX11 |
| | | OGT | MMP25 | MYOF |
| | | OR52A2/PSGR | MMP26 | NAALADL2 |
| | | PALM3 | MNX1 | NEAT1 |
| | | PCA3 | MSMB | NKAIN1 |
| | | PCSK6 | MX11 | NLRP3 |
| | | PDLIM5 | MYOF | OGT |
| | | PECI | NAALADL2 | OR52A2/PSGR |
| | | PPAP2A | NEAT1 | PALM3 |
| | | PPFIA2 | NKAIN1 | PCA3 |
| | | PPP1R12B | NLRP3 | PCSK6 |
| | | PSTPIP1 | OGT | PDLIM5 |
| | | PTN | OR52A2/PSGR | PECI |
| | | PTPRC | PALM3 | PPAP2A |
| | | PVT1 | PCA3 | PPFIA2 |
| | | RAB17 | PCSK6 | PPP1R12B |
| | | RIOK3 | PDLIM5 | PSTPIP1 |
| | | RNF157 | PECI | PTN |
| | | RP11-244H18.1/P712P | PPAP2A | PTPRC |
| | | RP11-97012.7 | PPFIA2 | PVT1 |
| | | RPL18A | PPP1R12B | RAB17 |
| | | RPL23AP53 | PSTPIP1 | RIOK3 |
| | | RPLP2 | PTN | RNF157 |
| | | RPS10 | PTPRC | RP11-244H18.1/P712P |
| | | RPS11 | PVT1 | RP11-97012.7 |
| | | SACM1L | RAB17 | RPL18A |
| | | SChLAP1 | RIOK3 | RPL23AP53 |
| | | SEC61A1 | RNF157 | RPLP2 |
| | | SERPINB5/Maspin | RP11-244H18.1/P712P | RPS10 |
| | | SFRP4 | RP11-97012.7 | RPS11 |
| | | SIM2.long | RPL18A | SACM1L |
| | | SIM2.short | RPL23AP53 | SChLAP1 |
| | | SIRT1 | RPLP2 | SEC61A1 |
| | | SLC12A1 | RPS10 | SERPINB5/Maspin |
| | | SLC43A1 | RPS11 | SFRP4 |
| | | SLC4A1 S | SACM1L | SIM2.long |
| | | SMAP1 ex 7-8 | SChLAP1 | SIM2.short |
| | | SMIM1 | SEC61A1 | SIRT1 |
| | | SNCA | SERPINB5/Maspin | SLC12A1 |
| | | SNORA20 | SFRP4 | SLC43A1 |
| | | SPINK1 | SIM2.long | SLC4A1 S |
| | | SPON2 | SIM2.short | SMAP1 ex 7-8 |
| | | SRSF3 | SIRT1 | SMIM1 |
| | | SSPO | SLC12A1 | SNCA |
| | | SSTR1 | SLC43A1 | SNORA20 |
| | | ST6GALNAC1 | SLC4A1 S | SPINK1 |
| | | STEAP2 | SMAP1 ex 7-8 | SPON2 |
| | | STEAP4 | SMIM1 | SRSF3 |
| | | STOM | SNCA | SSPO |
| | | SULF2 | SNORA20 | SSTR1 |
| | | SULT1A1 | SPINK1 | ST6GALNAC1 |
| | | SYNM | SPON2 | STEAP2 |
| | | TBP | SRSF3 | STEAP4 |
| | | TDRD | SSPO | STOM |
| | | TERF2IP | SSTR1 | SULF2 |
| | | TERT | ST6GALNAC1 | SULT1A1 |
| | | TFDP1 | STEAP2 | SYNM |
| | | TIMP4 | STEAP4 | TBP |
| | | TMCC2 | STOM | TDRD |
| | | TMEM45B | SULF2 | TERF2IP |
| | | TMEM47 | SULT1A1 | TERT |
| | | TMEM86A | SYNM | TFDP1 |
| | | TMPRSS2/ERG fusion | TBP | TIMP4 |
| | | TRPM4 | TDRD | TMCC2 |
| | | TWIST1 | TERF2IP | TMEM45B |
| | | UPK2 | TERT | TMEM47 |
| | | VAX2 | TFDP1 | TMEM86A |
| | | VPS13A | TIMP4 | TMPRSS2/ERG fusion |
| | | ZNF577 | TMCC2 | TRPM4 |
| | | | TMEM45B | TWIST1 |
| | | | TMEM47 | UPK2 |
| | | | TMEM86A | VAX2 |
| | | | TMPRSS2/ERG fusion | VPS13A |
| | | | TRPM4 | ZNF577 |
| | | | TWIST1 | |
| | | | UPK2 | |
| | | | VAX2 | |
| | | | VPS13A | |
| | | | ZNF577 | |

### EXAMPLES

### Example 1

### Patient Population and Characteristics

The full Movember GAP1 urine cohort comprises of 1,257 first-catch post-DRE, pre-TRUS biopsy urine samples collected between 2009 and 2015 from urology clinics at multiple sites. Samples within the Movember cohort that were analysed for both methylation and cf-RNA were eligible for selection for model development in the current study (n=207).

Exclusion criteria for model development included a recent prostate biopsy or trans-urethral resection of the prostate (<6 weeks) and metastatic disease (confirmed by a positive bone-scan or PSA>100 ng/mL), resulting in a cohort of 197 samples, deemed the ExoMeth cohort (Table 2). The samples analysed in the ExoMeth cohort were collected from the Norfolk and Norwich University Hospital (NNUH, Norwich, UK) and St. James's Hospital (SJH, Dublin, Republic of Ireland). Sample collections and processing were ethically approved in their country of origin: NNUH samples by the East of England REC (n=181), Dublin samples by St. James's Hospital (n=16).

### Sample Processing and Analysis

Urine samples were processed according to the Movember GAP1 standard operating procedure (Supplementary Methods). Hypermethylation at the 5′-regulatory regions of six genes (GSTP1, SFRP2, IGFBP3, IGFBP7, APC and PTSG2) in urinary cell-pellet DNA was assessed using quantitative methylation-specific PCR as described by O'Reilly et al (2019) [30]. Cell-free mRNA was isolated and quantified from urinary extracellular vesicles using NanoString technology, with 167 gene-probes (ExoRNA column of Table 1), as described in Connell et al (2019) [31], with the modification that NanoString data were normalised according to NanoString guidelines using NanoString internal positive controls, and log 2 transformed. Clinical variables that were considered are serum PSA, age at sample collection, DRE impression and urine volume collected.

### Statistical Analysis

All analyses, model construction and data preparation were undertaken in R version 3.5.3 [51], and unless otherwise stated, utilised base R and default parameters. All data and code required to reproduce these analyses can be found at https://github.com/UEA-Cancer-Genetics-Lab/ExoMeth.

### Example 2—ExoMeth Model Design

### Feature Selection

In total 177 variables available for prediction (cf-RNA (n=167), methylation (n=6) and clinical variables (n=4). For full list see Table 1), making feature selection a key task for minimising model overfitting and increasing the robustness of trained models. To avoid dataset-specific features being positively selected [61] we implemented a robust feature selection workflow utilising the Boruta algorithm [62] and bootstrap resampling. Boruta is a random forest-based algorithm that iteratively compares feature importance against random predictors, deemed “shadow features”. Features that perform significantly worse compared to the maximally performing shadow feature at each permutation, (p≤0.01, calculated by Z-score difference in mean accuracy decrease) are consecutively dropped until only confirmed, stable features remain.

Boruta was applied on 1,000 datasets generated by resampling with replacement. Features were only positively selected for model construction when confirmed as stable features in ≥90% of resampled Boruta runs.

### Comparator Models

To evaluate potential clinical utility, additional models were trained as comparators using subsets of the available variables across the patient population: a clinical standard of care (SoC) model was trained by incorporating age, PSA, T-staging and clinician DRE impression; a model using only the available DNA methylation probes (Methylation, n=6); and a model only using NanoString gene-probe information (NanoString, n=167). The fully integrated ExoMeth model was trained by incorporating information from all of the above variables (n=177). Each set of variables for comparator models were independently selected via the bootstrapped Boruta feature selection process described above to select the most optimal subset of variables possible for each predictive model.

### Model Construction

All models were trained via the random forest algorithm [63], using the randomForest package [64] with default parameters except for: resampling without replacement and 401 trees being grown per model. Risk scores from trained models are presented as the out-of-bag predictions; the aggregated outputs from decision trees within the forest where the sample in question has not been included within the resampled dataset [63]. Bootstrap resamples were identical for feature selection and model training for all models and used the same random seed.

Models were trained on a modified continuous label, based on biopsy outcome and constructed as follows: samples were scored on a continuous scale (range: 0-1) according to Gleason score: where 0 represents no evidence of cancer, Gleason scores 6 & 3+4 are equal to 0.5 and Gleason scores ≥4+3 are set to 1. This recognises that two patients with the same Gleason scored TRUS-biopsy detected cancer will not share the exact same proportions of tumour pattern, or overall disease burden. This scale is solely used for model training and is not represented in any endpoint measurements, or for determining predictive ability and clinical utility.

### Statistical Evaluation of Model Predictivity

Area Under the Receiver-Operator Characteristic curve (AUC) metrics were produced using the package [65], with confidence intervals calculated via 1,000 stratified bootstrap resamples. Density plots of model risk scores, and all other plots were created using the ggplot2 package [66]. Cumming estimation plots and calculations were produced using the dabestr package [67] and 1,000 bootstrap resamples were used to visualise robust effect size estimates of model predictions.

Decision curve analysis (DCA) (34) examined the potential net benefit of using PUR-signatures in the clinic. Standardised net benefit (sNB) was calculated with the rmda package [69] and presented throughout our decision curve analyses as it is a more directly interpretable metric compared to net benefit [70]. In order to ensure DCA was representative of a more general population, the prevalence of Gleason scores within the ExoMeth cohort were adjusted via bootstrap resampling to match those observed in a population of 219,439 men that were in the control arm of the Cluster Randomised Trial of PSA Testing for Prostate Cancer (CAP) Trial [13], as described in Connell et al (2019). Briefly, of the biopsied men within this CAP cohort, 23.6% were Gs 6, 8.7% Gs 7 and 7.1% Gs≥8, with 60.6% of biopsies showing no evidence of cancer. These ratios were used to perform stratified bootstrap sampling with replacement of the Movember cohort to produce a “new” dataset of 197 samples with risk scores from each comparator model. sNB was then calculated for this resampled dataset, and the process repeated for a total of 1,000 resamples with replacement. The mean sNB for each risk score and the “treat-all” options over all iterations were used to produce the presented figures to account for variance in resampling. Net reduction in biopsies, based on the adoption of models versus the default treatment option of undertaking biopsy in all men with PSA≥4 ng/mL was calculated as:

$${Biopsy}_{NetReduction} = ( N {B}_{Model} - N {B}_{All} ) \times \frac{1 - Threshold}{Threshold}$$

Where the decision threshold (Threshold) is determined by accepted patient/clinician risk [68]. For example, a clinician may accept up to a 25% perceived risk of cancer before recommending biopsy to a patient, equating to a decision threshold of 0.25.

### Example 3—ExoMeth Data

### The ExoMeth Development Cohort

Linked methylation and transcriptomic data were available for 197 patients within the Movember GAP1 cohort, with the majority originating from the NNUH and forming the ExoMeth development cohort (Table 2). The proportion of Gleason ≥7 disease in the ExoMeth cohort was 49%.

TABLE 2
Characteristics of the ExoMeth development cohort.

| | Biopsy Negative: | | Biopsy Positive | |
|---|---|---|---|---|
| Collection Centre: | | | | |
| NNUH, n (%) | 68 | (88) | 113 | (94) |
| SJH, n (%) | 9 | (12) | 7 | (6) |
| Age: | | | | |
| minimum | 42.00 | | 53.00 | |
| median (IQR) | 66.00 | (59.00, 71.00) | 69.50 | (65.00, 76.00) |
| mean (sd) | 65.70 ± 8.53 | | 69.97 ± 7.44 | |
| maximum | 82.00 | | 86.00 | |
| PSA: | | | | |
| minimum | 0.20 | | 3.60 | |
| median (IQR) | 6.70 | (4.20, 8.80) | 10.05 | (6.90, 18.20) |
| mean (sd) | 7.44 ± 5.59 | | 17.50 ± 18.82 | |
| maximum | 30.30 | | 95.90 | |
| Prostate Size | | | | |
| (DRE Estimate): | | | | |
| Small, n (%) | 14 | (18) | 12 | (10) |
| Medium, n (%) | 29 | (38) | 56 | (47) |
| Large, n (%) | 22 | (29) | 37 | (31) |
| Unknown, n (%) | 12 | (16) | 15 | (12) |
| Gleason Score: | | | | |
| ≥8, n (%) | 0 | (0) | 31 | (26) |
| 0, n (%) | 77 | (100) | 0 | (0) |
| 3 + 4, n (%) | 0 | (0) | 42 | (35) |
| 4 + 3, n (%) | 0 | (0) | 23 | (19) |
| 6, n (%) | 0 | (0) | 24 | (20) |
| Biopsy_Result | | | | |
| Biopsy Negative | 77 | (100) | 0 | (0) |
| Biopsy Positive | 0 | (0) | 120 | (100) |

### Feature Selection and Model Development

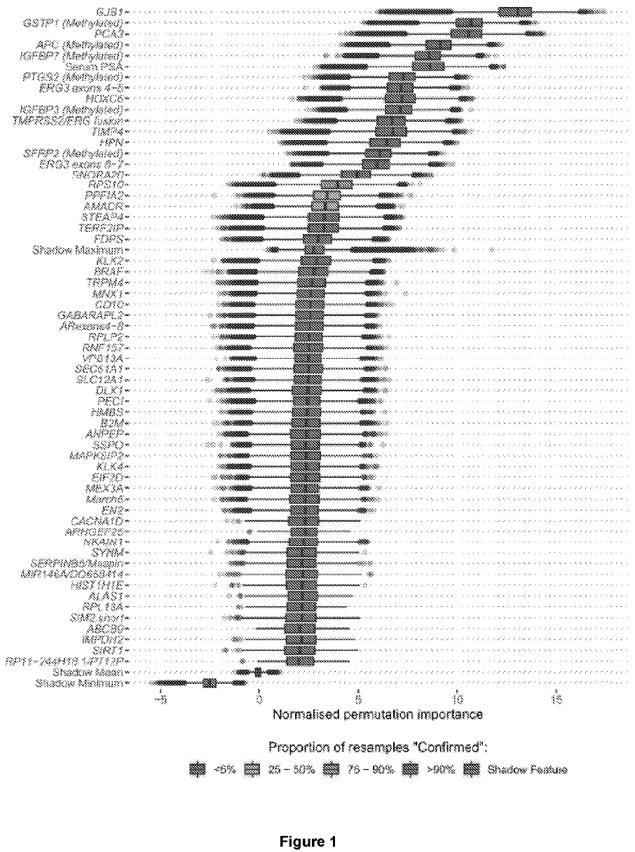

Using a robust feature selection framework four models were produced in total; a standard of care (SoC) model using only clinical information (age and PSA), a model using only methylation data (Methylation, 6 genes), a model using only cf-RNA information (ExoRNA, 12 gene-probes) and the integrated model, deemed ExoMeth (16 variables) (Table 3). The ExoMeth model is a multivariable risk prediction model incorporating clinical, methylation and cf-RNA variables. When the resampling strategy was applied for feature reduction using Boruta, 16 variables were selected for the ExoMeth model. Each of the retained variables were positively selected in every resample and notably included information from clinical, methylation and cf-RNA variables (FIG. 1). Full resample-derived Boruta variable importances for the SoC, Methylation and ExoRNA comparator models can be seen in Supplementary FIGS. 1-3, respectively.

In the SoC comparator model only PSA and age were selected as important predictors. All methylation probes were selected as important in both the independent Methylation model and the ExoMeth models (Table 3). 12 NanoString gene-probes were selected for the ExoRNA model, notably containing both variants of the ERG gene-probe and TMPRSS2/ERG fusion gene-probe, alongside PCA3. All features within the ExoMeth model were also selected in one of the comparator models.

TABLE 3
Boruta-derived features positively selected for each model. Features
are selected for each model by being confirmed as important for predicting
biopsy outcome, categorised as a modified ordinal variable by Boruta
in ≥90% of bootstrap resamples. Variables selected for the fully
integrated model (ExoMeth) are in the highlighted column; for example;
Age is selected within the SoC model, but not in ExoMeth.

| | Models: | | | |
|---|---|---|---|---|
| | SoC | Methylation | ExoRNA | ExoMeth |
| Clinical | Serum PSA | — | — | Serum PSA |
| Parameters: | Age | — | — | — |
| | — | GSTP1 | — | GSTP1 |
| | — | APC | — | APC |
| Methylation | — | SFRP2 | — | SFRP2 |
| Targets: | — | IGFBP3 | — | IGFBP3 |
| | — | IGFBP7 | — | IGFBP7 |
| | — | PTGS2 | — | PTGS2 |
| | — | — | AMACR | — |
| | — | — | ERG exons 4-5 | ERG exons 4-5 |
| | — | — | ERG exons 6-7 | ERG exons 6-7 |
| | — | — | GJB1 | GJB1 |
| | — | — | HOXC6 | HOXC6 |
| Transcript | — | — | HPN | HPN |
| Targets | — | — | PCA3 | PCA3 |
| | — | — | PPFIA2 | — |
| | — | — | RPS10 | — |
| | — | — | SNORA20 | SNORA20 |
| | — | — | TIMP4 | TIMP4 |
| | — | — | TMPRSS2/ERG fusion | TMPRSS2/ERG fusion |

### ExoMeth Predictive Ability

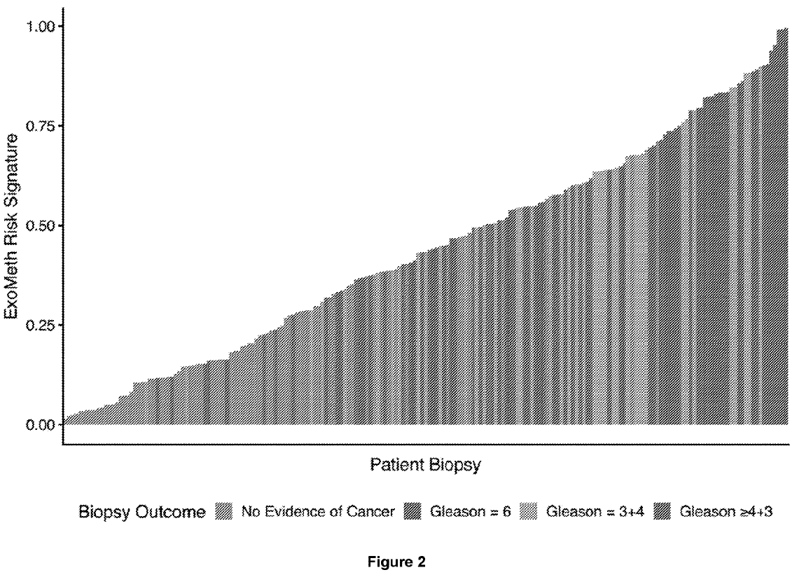

As ExoMeth Risk Score (range 0-1) increased, the likelihood of high-grade disease being detected on biopsy was significantly greater (Proportional odds ratio=2.04 per 0.1 ExoMeth increase, 95% CI: 1.78-2.35; ordinal logistic regression, FIG. 2). The median ExoMeth risk score was 0.83 for metastatic patients (n=10). These were excluded from model training and can be considered as a positive control. One metastatic sample had a lower than expected ExoMeth score of 0.55: where no methylation was quantified for this sample, which may reflect a technical failure of the sample.

TABLE 4
AUC of all trained models (ExoMeth) for detecting outcomes of an initial
biopsy for varying clinically significant thresholds. Brackets show 95%
confidence intervals of the AUC, calculated from 1,000 stratified bootstrap
resamples. Input variables for each model are detailed in Table 1.

| Initial biopsy outcome: | SoC | Methylation | ExoRNA | ExoMeth |
|---|---|---|---|---|
| Gleason ≥4 + 3: | 0.75 (0.67-0.82) | 0.77 (0.68-0.85) | 0.74 (0.66-0.81) | 0.81 (0.75-0.87) |
| Gleason ≥3 + 4: | 0.73 (0.65-0.79) | 0.78 (0.71-0.84) | 0.81 (0.75-0.87) | 0.89 (0.84-0.93) |
| Any Cancer | 0.70 (0.62-0.77) | 0.73 (0.66-0.79) | 0.86 (0.81-0.91) | 0.91 (0.87-0.95) |

ExoMeth was superior to all other models, returning an AUC of 0.89 (95% CI: 0.84-0.93) for Gleason ≥3+4 and 0.81 (95% CI: 0.75-0.87) for Gleason ≥4+3 (Table 4).

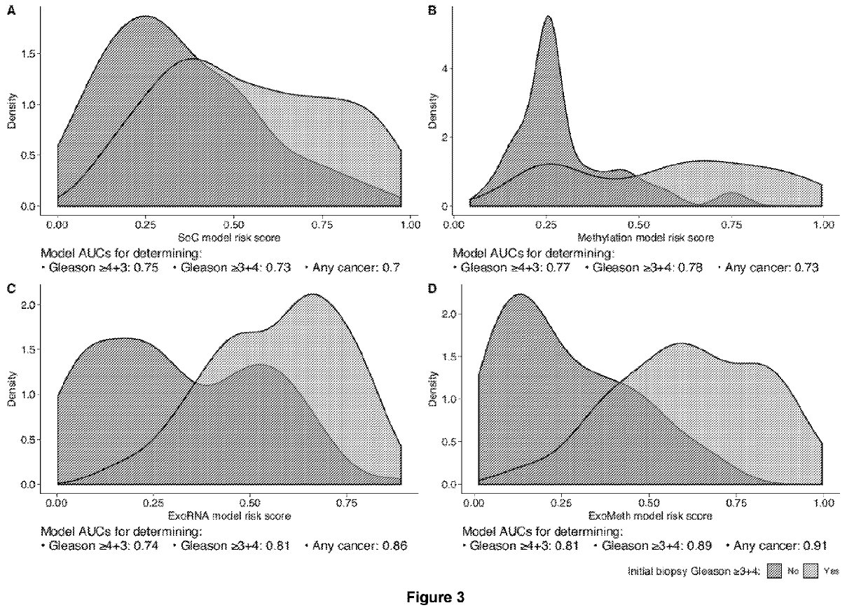

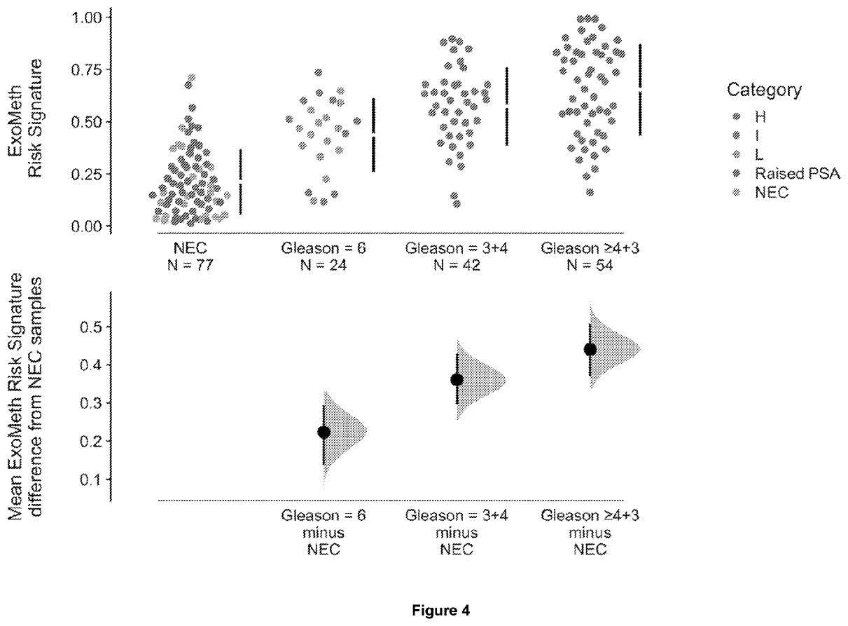

As revealed by the distributions of risk scores and AUC, ExoMeth achieved a better discrimination of Gleason ≥3+4 disease from other outcomes when compared to any of the other models (ExoMeth all p<0.01 bootstrap test, 1,000 resamples, FIG. 3). The SoC model, whilst returning respectable AUCs, would misclassify more men with indolent disease as warranting further investigation than all other models (FIG. 3A), for example, to classify 90% of Gleason 7 men correctly, an SoC risk score of 0.237 would misclassify 65% of men with less significant disease. The methylation comparator model improves upon SoC, by drawing the risk distribution of Gs<6 men into a more pronounced peak but featured a bimodal risk score distribution extending to higher-risk men; almost 50% of men with Gs≥ 3+4 have risk scores equal to benign patients (FIG. 3B). The opposite occurred in the ExoRNA comparator model exhibited a broad bimodal distribution for lower-risk men (FIG. 3C). This discriminatory ability of the ExoMeth model over all comparators was improved when biopsy outcomes are considered as biopsy negative, Gleason 6 or 3+4, or Gleason ≥4+3 (Supplementary FIG. 4).

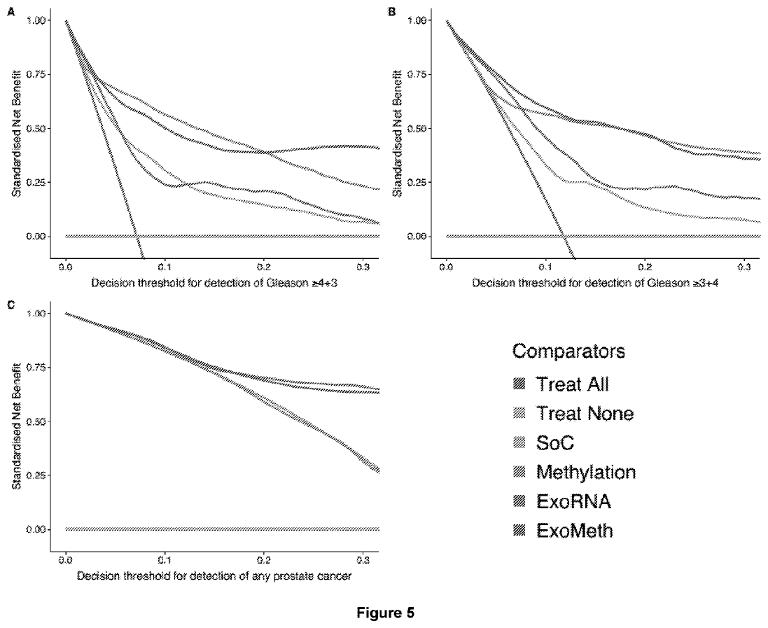

Resampling of ExoMeth predictions via estimation plots allowed for comparisons of mean ExoMeth signatures between groups (1,000 bias-corrected and accelerated bootstrap resamples, FIG. 4). The mean ExoMeth differences between patients with no evidence of cancer were: Gleason 6=0.22 (95% CI: 0.14-0.30), Gleason 3+4=0.36 (95% CI: 0.28-0.42) and Gleason ≥4+3=0.44 (95% CI: 0.37-0.51). Notably, there were no differences in ExoMeth risk signatures of patients with a raised PSA but negative for cancer on biopsy and men with no evidence of cancer (mean difference=0.03 (95% CI: 0.05-0.10), FIG. 4, Supplementary FIG. 5).

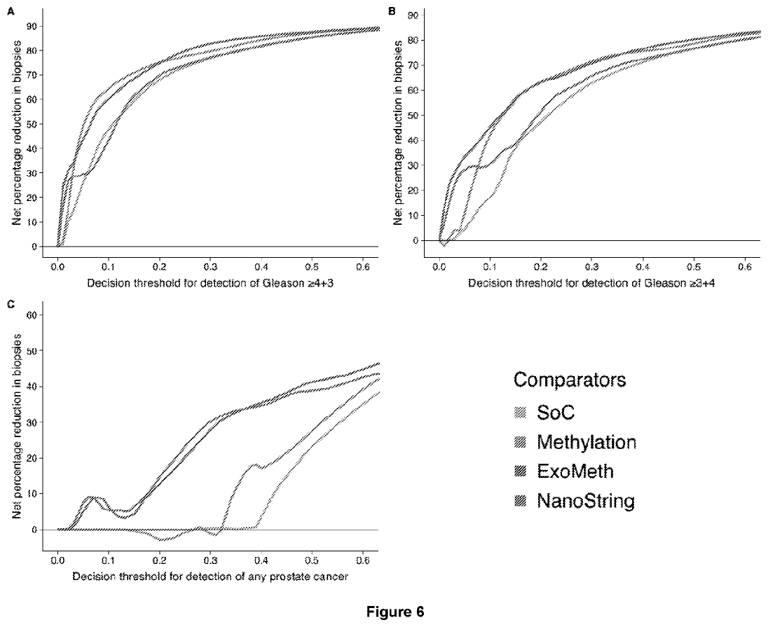

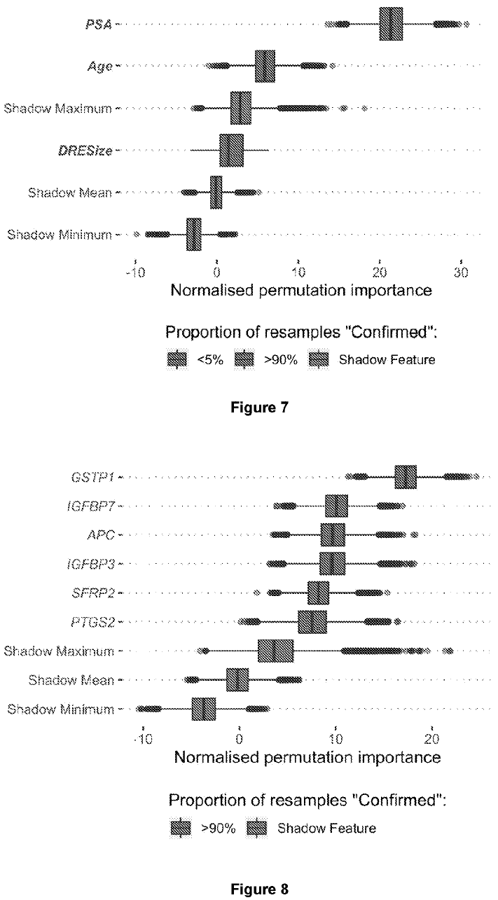

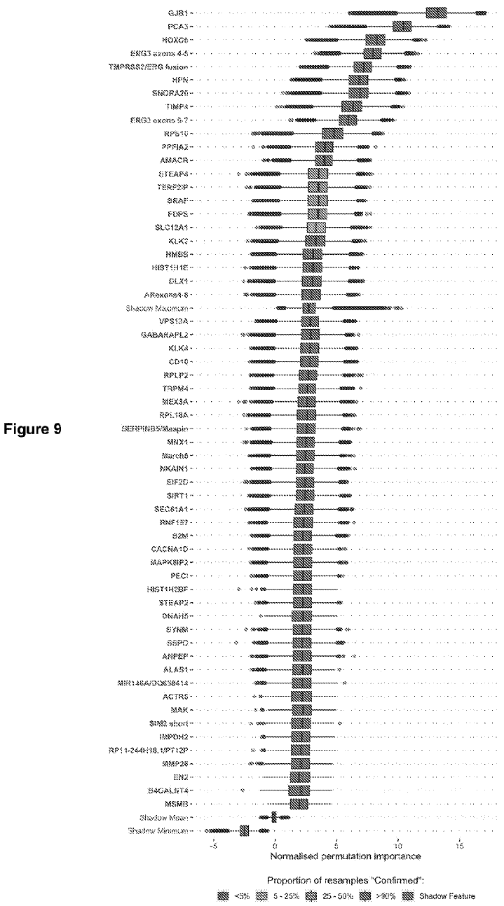

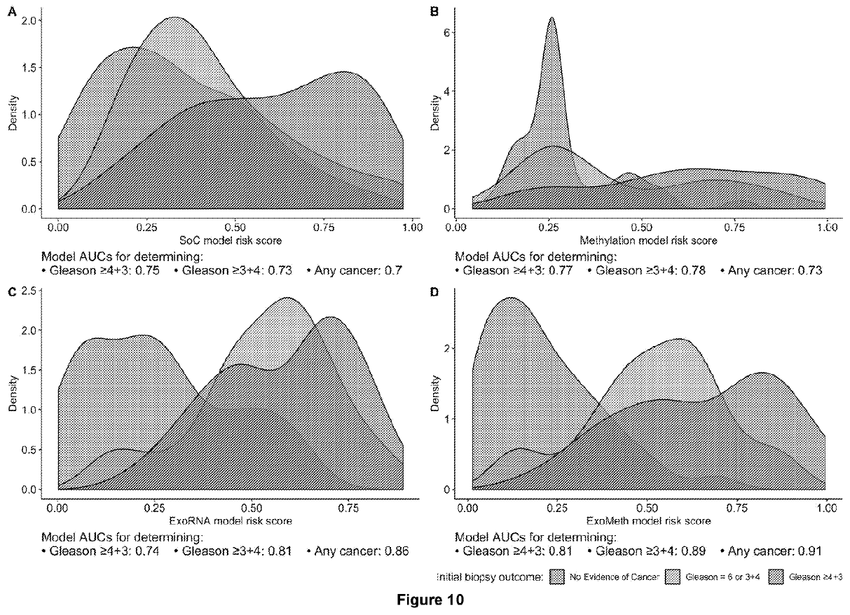

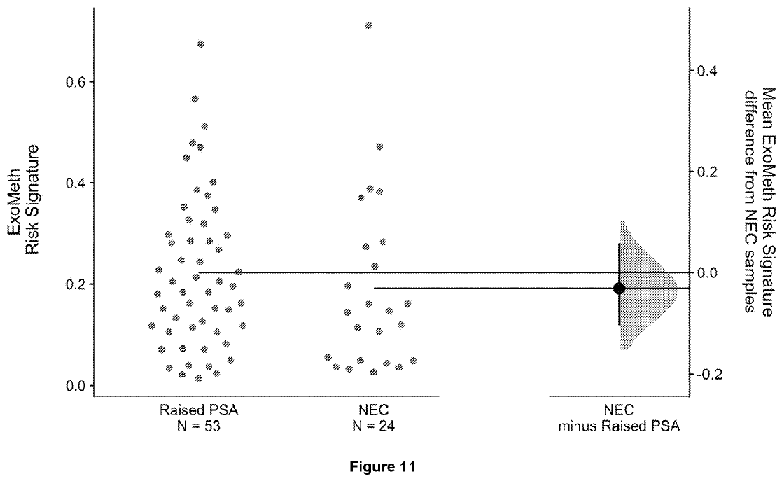

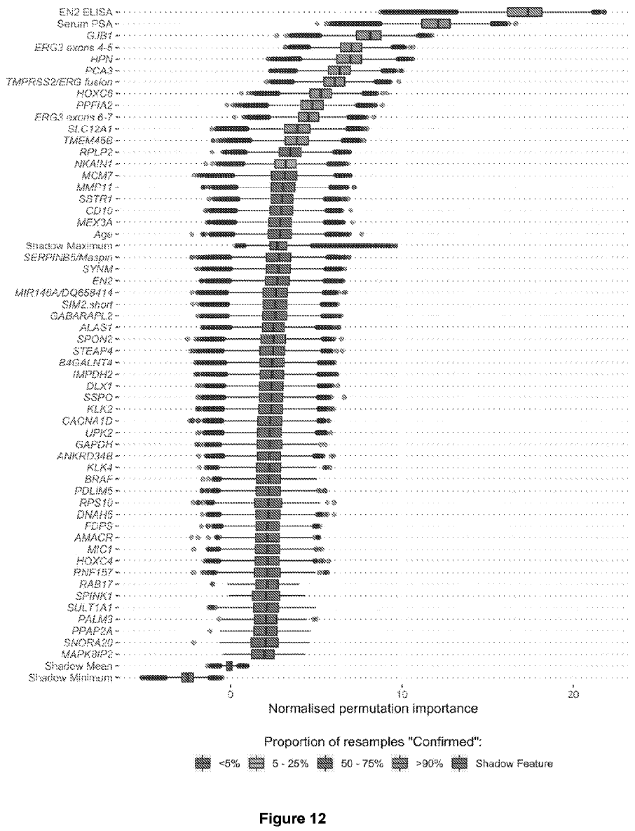

Decision curve analysis examined the net benefit of adopting ExoMeth in a population of patients suspected with prostate cancer and to have a PSA level suitable to trigger biopsy (≥4 ng/mL). The biopsy of men based upon their ExoMeth risk score consistently provided a net benefit over current standards of care across all decision thresholds examined and was the most consistent amongst all comparator models across a range of clinically relevant endpoints for biopsy (FIG. 5). Of the patients with Gs≥7 disease, 95% had an ExoMeth risk score ≥0.283. At a decision threshold of 0.25, ExoMeth could result in up to 66% fewer unnecessary biopsies of men presenting with a suspicion of prostate cancer, without missing substantial numbers of men with aggressive disease, whilst if Gleason ≥4+3 were considered the threshold of clinical significance, the same decision threshold of 0.25 could save 79% of men from receiving an unnecessary biopsy (FIG. 6).

### Example 4—ExoMeth Results

The accurate discrimination of disease state in men prior to a confirmatory initial biopsy would mark a significant development and impact large numbers of men suspected of harbouring prostate cancer. Up to 75% of men with a raised PSA (4 ng/mL) are negative for prostate cancer on biopsy [4,13,52]. This has resulted in concentrated research efforts to address this problem non-invasively, and resulting in the development of several biomarker panels capable of detecting Gleason ≥3+4 disease with superior accuracy to current clinically implemented methods [19, 20, 21, 31]. However, in each of these examples, only a single quantification method or biological process is assayed and with the molecular heterogeneity of prostate cancer considered [53], a more holistic approach is necessary.

It is becoming apparent from published data that urine can contain a wealth of useful cancer biomarkers within RNA, DNA, cell-free DNA, DNA methylation and proteins [22, 30, 31, 54, 55]. However, the analyses presented here are, to the author's knowledge, the first attempt to integrate such biomarker information within the same samples for the detection of prostate cancer prior to biopsy. There has recently been reported that a combination of miRNA and methylation markers can be used to predict outcome following radical prostatectomy [56]. Our results show an improved diagnostic marker can be produced from the synergistic relationship of information derived from different urine fractions in men suspected to have prostate cancer. The methylation of six previously identified genes [30] was quantified via methylation specific qPCR, whilst the transcript levels of 167 cell-free mRNAs were quantified using NanoString technology. The final model integrating this information with serum PSA levels was deemed ExoMeth. Markers selected for the model include well known genes associated with prostate cancer and proven in other diagnostic tests, such as HOXC6 [20], PCA3 [19] and the TMPRSS2/ERG gene fusion [57]. ExoMeth additionally incorporated GJB1 as the most important variable for predicting biopsy outcome. Whilst GJB1 is known to be a prognostic marker for favourable outcome in renal cancers, there is no evidence of its use as a diagnostic biomarker in prostate cancer [58, 59].

ExoMeth was able to correctly predict the presence of significant prostate cancer on biopsy with an AUC of 0.89, representing a significant uplift when compared to other published tests (AUCs for Gs≥7: PUR=0.77 [31], ProCUrE=0.73 [22], ExoDX Prostate IntelliScore=0.77 [21], SelectMDX=0.78 [20], epiCaPture Gs≥4+3 AUC=0.73 [30]). Furthermore, ExoMeth resulted in accurate predictions even when serum PSA levels alone were inaccurate; where patients with a raised PSA but negative biopsy result possessed similar ExoMeth scores as clinically benign men, whilst still able to discriminate between Gleason grades (FIG. 4). These are men that would be unnecessarily subjected to biopsy by current guidelines. Of the three patients with no evidence of cancer on biopsy with an ExoMeth risk score >0.55, two were positive for the TMPRSS2/ERG fusion transcript in NanoString analyses (data not shown), implying that PCa may have been missed and re-biopsy may be necessary [60].

Whilst every step has been taken to robustly develop ExoMeth to minimise potential overfitting and bias through extensive bootstrap resampling and the use of out-of-bag predictions, ExoMeth nonetheless was developed on a small dataset and requires validation in an independent cohort before its use a clinical marker can be considered. Additionally, as MP-MRI can misrepresent disease state in patients, even when rigorous protocols are implemented [15] the clinical utility of supplementing MP-MRI with ExoMeth needs to be assessed. For many men harbouring indolent prostate cancer, ExoMeth could greatly impact their experience of prostate cancer care when compared to current clinical pathways.

### Example 5—ExoGrail Model Design

### Feature Selection

With many variables available for prediction (n=172, NanoString, EN2 ELISA and clinical variables. For full list see Table 1), feature selection was a key task for minimising overfit and increasing the robustness of trained models. However, applying feature selection to a complete dataset can result in dataset-specific features being positively selected [61]. With this considered, we implemented a robust feature selection workflow utilising the Boruta algorithm [62] and bootstrap resampling. Boruta is a random forest-based algorithm that iteratively compares feature importance against random predictors, deemed “shadow features”. These shadow features are created by permutation of original features rather than arbitrary “randomness”. Features that perform significantly worse compared to the maximally performing shadow feature at each permutation, (p≤0.01, calculated by Z-score difference in mean accuracy decrease) are consecutively dropped until only confirmed, stable features remain.

Boruta is implemented within a bootstrap resampling loop here, with the normalised permutation featured importance aggregated over 1,000 resamples with replacement. Features were only positively selected for model construction when confirmed in ≥90% of resampled Boruta runs.

### Model Construction

To evaluate potential clinical utility, additional models were trained as comparators using subsets of the available variables across the patient population: a clinical standard of care (SOC) model was trained by incorporating age, PSA, T-staging and clinician DRE impression; a model using only the EN2 ELISA result (EN2); and a model only using NanoString gene-probe information (ExoRNA). The fully integrated ExoGrail model was trained by incorporating information from all of the above variables. Each set of variables for comparator models were independently selected via the bootstrapped Boruta feature selection process described above to select the most optimal subset of variables possible for each predictive model.

All models were trained via the random forest algorithm [62], using the randomForest package [63], with default parameters except for resampling without replacement and 401 trees grown per model. Risk scores from trained models are presented as the out-of-bag predictions; the aggregated outputs from decision trees within the forest where the sample in question has not been included within the resampled dataset [62]. Bootstrap resamples were identical between the comparators for feature selection and model training and used the same random seed.

Models were trained on a modified continuous label, based on biopsy outcome and constructed as follows: samples were first categorised as an ordinal variable according to the biopsy Gleason score as either; no evidence of cancer (NEC), lower-grade cancer—Gleason 6 & 3+4 (LC), and higher-grade cancer—Gleason ≥4+3 (HC). In order to recognise that no two patients with the same Gleason graded TRUS-biopsy detected cancer will share the exact same proportions of tumour pattern, or overall disease burden, this ordinal variable was further treated as a continuous predictor, where 0 represents NEC, 0.5 the LC label and 1 the HC label of aggressive disease Gleason ≥4+3.

### Statistical Evaluation of Model Predictivity

Area Under the Receiver-Operator Characteristic curve (AUROC) analyses were produced using the pROC package [64], with confidence intervals calculated via 2,000 stratified bootstrap resamples. Density plots of model risk scores, and all other plots used the ggplot2 package [65]. Cumming estimation plots and calculations were produced using the dabestr package [66] and 5,000 bootstrap resamples to visualise robust effect size estimates of model predictions.

Decision curve analysis (DCA) [67] examined the potential net benefit of using PUR-signatures in the clinic.

Standardised net benefit (sNB) was calculated with the rmda package [68] and presented throughout our decision curve analyses as it is a more directly interpretable metric compared to net benefit [69]. In order to ensure DCA was representative of a more general population, the prevalence of Gleason grades within the Movember cohort were adjusted via bootstrap resampling to match that observed in a population of 219,439 men that were in the control arm of the Cluster Randomised Trial of PSA Testing for Prostate Cancer (CAP) Trial [70], similarly to those methods previously reported in Connell et al (2019). Briefly, of the biopsied men within this CAP cohort, 23.6% were Gs 6, 8.7% Gs 7 and 7.1% Gs 8 or greater, with 60.6% of biopsies showing no evidence of cancer. These ratios were used to perform stratified bootstrap sampling with replacement of the Movember cohort to produce a “new” dataset of 150 samples with risk scores from each comparator model. sNB was then calculated for this resampled dataset, and the process repeated for a total of 500 resamples with replacement. The mean sNB for each risk score and the “treat-all” options over all iterations were used to produce the presented figures to account for variance in resampling. Net reduction in biopsies, based on the adoption of models versus the default treatment option of undertaking biopsy in all men with PSA≥3 ng/mL was calculated as:

$${Biopsy}_{NetReduction} = ( N {B}_{Model} - N {B}_{All} ) \times \frac{1 - Threshold}{Threshold}$$

### Example 6—ExoGrail Results

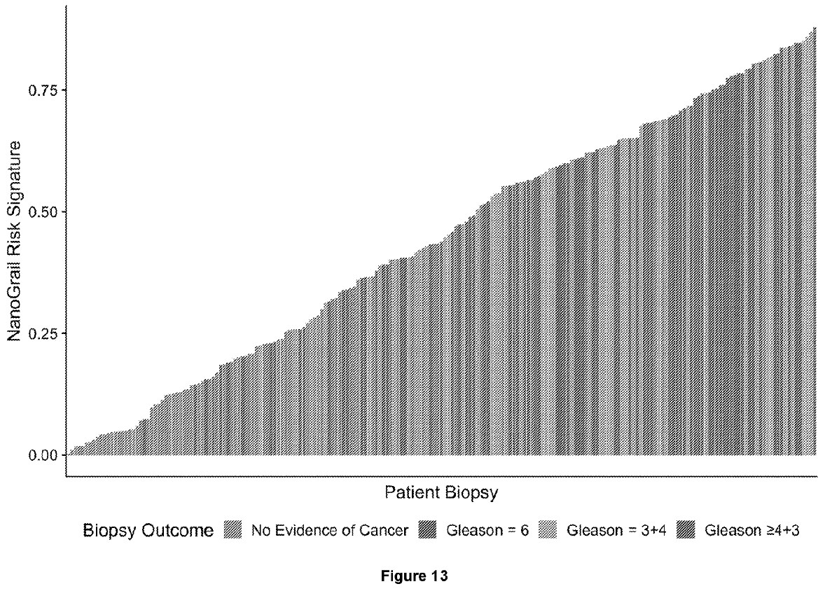

As ExoGrail Risk Score (range 0-1) increased, the likelihood of high-grade disease being detected on biopsy was significantly greater (Proportional odds ratio=2.21 per 0.1 ExoGrail increase, 95% CI: 1.91-2.59; ordinal logistic regression, FIG. 13). The median ExoGrail risk score was 0.7645677 for metastatic patients (n=11). These were excluded from model training and can be considered as a positive control.

TABLE 5
Boruta-derived features positively selected for each model. Features
are selected for each model by being confirmed as important for predicting
biopsy outcome, categorised as a modified ordinal variable by Boruta
in ≥90% of bootstrap resamples. Variables selected for the fully
integrated model (ExoGrail) are in the highlighted column; for example;
Age is selected within the SoC model, but not in ExoGrail.

| | Models: | | | |
|---|---|---|---|---|
| | SoC | EN2 | ExoRNA | ExoGrail |
| Clinical | Serum PSA | — | — | Serum PSA |
| Parameters: | Age | — | — | — |
| EN2 | — | EN2 Protein | — | EN2 Protein |
| | — | — | ERG exons 4-5 | ERG exons 4-5 |
| Transcript | — | — | ERG exons 6-7 | ERG exons 6-7 |
| Targets | — | — | GJB1 | GJB1 |
| | — | — | HOXC6 | HOXC6 |
| | — | — | HPN | HPN |
| | — | — | NKAIN1 | — |
| | — | — | PCA3 | PCA3 |
| | — | — | PPFIA2 | PPFIA2 |
| | — | — | RPLP2 | — |
| | — | — | TMEM45B | TMEM45B |
| | — | — | TMPRSS2/ERG fusion | TMPRSS2/ERG fusion |
| | — | — | | SLC12A1 |

TABLE 6
AUC of all trained models (ExoGrail) for detecting outcomes of an initial
biopsy for varying clinically significant thresholds. Brackets show 95%
confidence intervals of the AUC, calculated from 1,000 stratified bootstrap
resamples. Input variables for each model are detailed in Table 1.

| Initial biopsy outcome: | SoC | EN2 | ExoRNA | ExoGrail |
|---|---|---|---|---|
| Gleason =4 + 3: | 0.77 (0.69-0.85) | 0.81 (0.73-0.88) | 0.67 (0.60-0.75) | 0.84 (0.78-0.90) |
| Gleason =3 + 4: | 0.72 (0.65-0.79) | 0.83 (0.77-0.88) | 0.77 (0.70-0.82) | 0.90 (0.86-0.94) |
| Any Cancer | 0.75 (0.68-0.82) | 0.81 (0.74-0.87) | 0.81 (0.75-0.87) | 0.89 (0.85-0.94) |

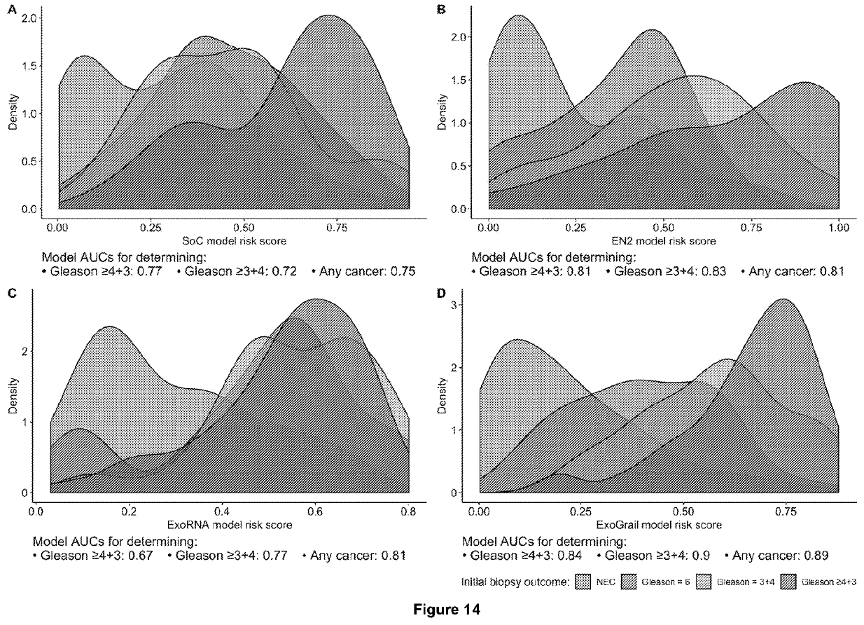

As revealed by the distributions of risk scores and AUC, ExoGrail achieved a better discrimination of Gleason ≥3+4 disease from other outcomes when compared to any of the other models (ExoGrail all p<0.01 bootstrap test, 1,000 resamples, FIG. 14).

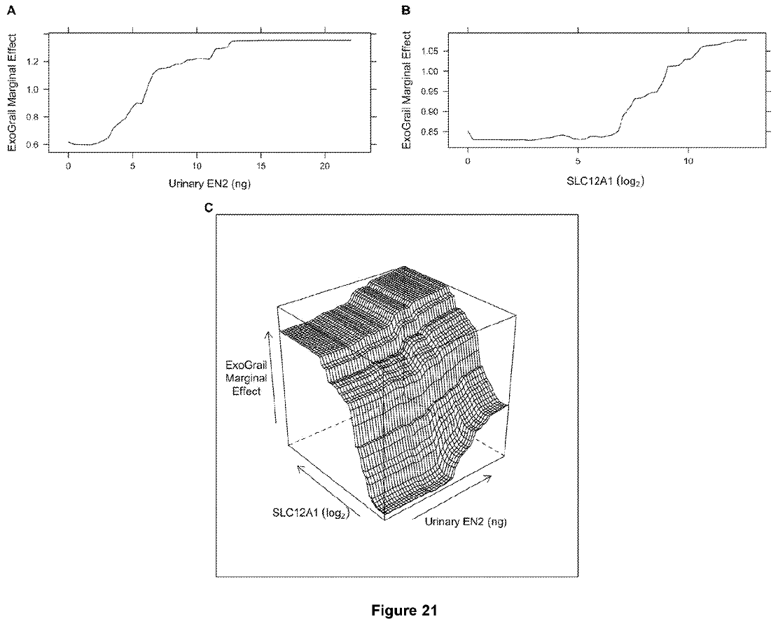

The SoC model, whilst returning respectable AUCs, displayed a realtive inability to clearly stratify disease status, and would cause large numbers of men to be inappropriately selected for further investigation (FIG. 14A). For example, to classify 90% of Gleason 7 men correctly, an SoC risk score of 0.251 would misclassify 64.5% of men with less significant, or no disease. The EN2 model detailed much clearer discrimination, though featured a biomodal distribution of patients without prostate cancer (FIG. 14B, green density plot), falsely identifying 51.4% of patients with low grade disease as warranting invasive followup (FIG. 14B). As more molecular markers were considered in the ExoRNA model, the bimodal distribution flattened and, despite attaining lower AUCs, ExoRNA could more accurately discriminate cancer from non-cancer than either the SoC or EN2 models (FIG. 14C). The greater discriminatory ability of the ExoGrail model when biopsy outcomes are considered as a binary Gleason ≥3+4 threshold can also be seen in FIG. 21.

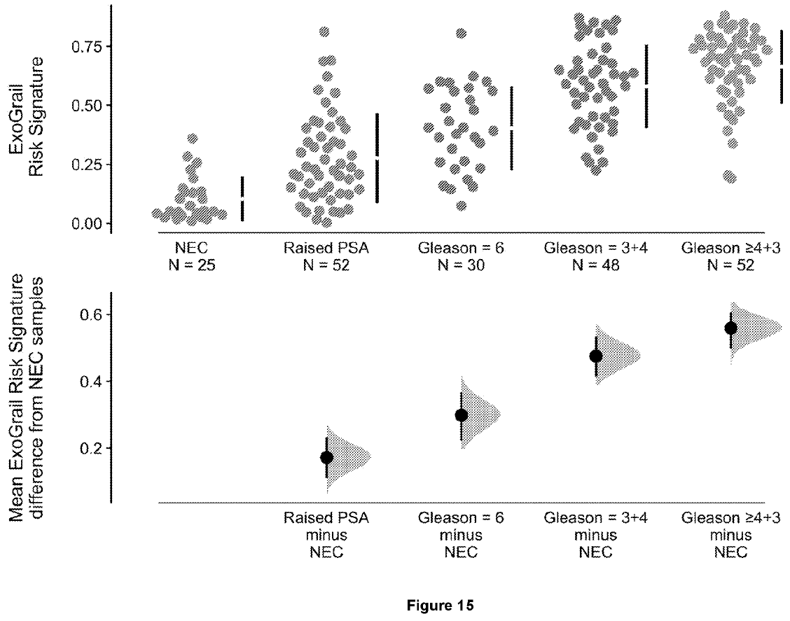

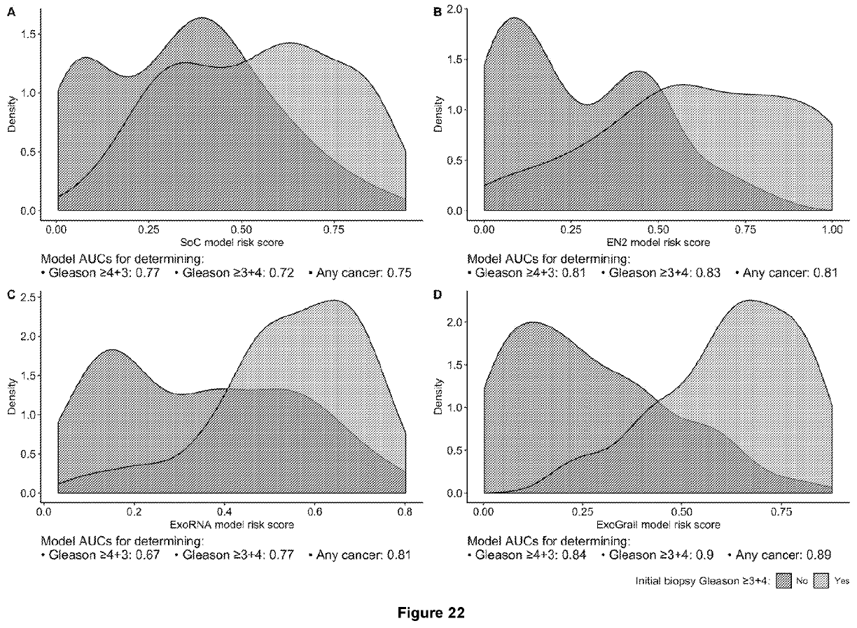

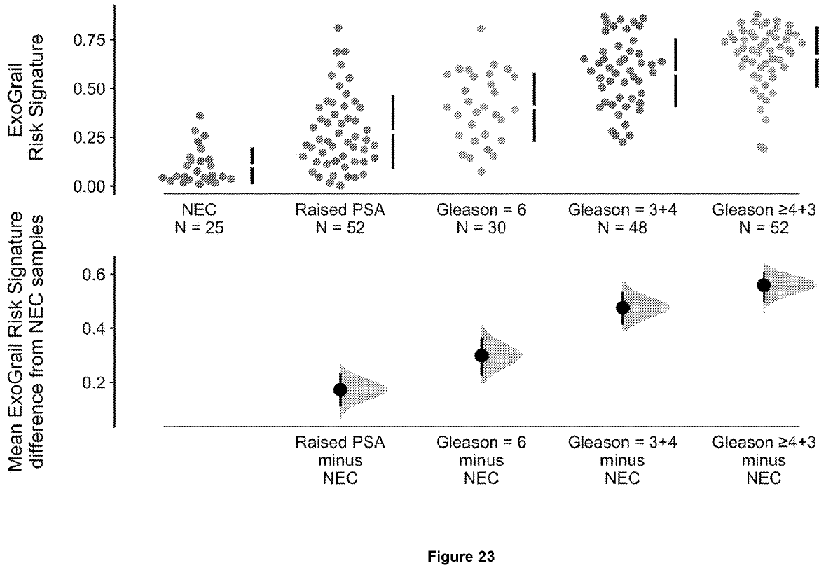

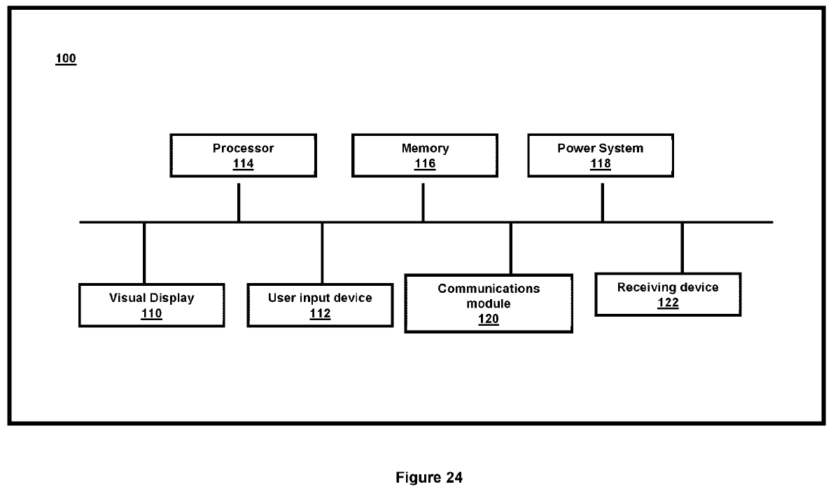

Resampling of ExoGrail predictions via estimation plots allowed for comparisons of mean ExoGrail signatures between groups (1,000 bias-corrected and accelerated bootstrap resamples, FIG. 15). The mean ExoGrail differences between patients with no evidence of cancer on biopsy were: Gleason 6=0.3 (95% CI: 0.22-0.37), Gleason 3+4=0.48 (95% CI: 0.41-0.53) and Gleason ≥4+3=0.56 (95% CI: 0.5-0.61). Interestingly, patients with no evidence of cancer had a lower ExoGrail risk score (mean difference=0.17 (95% CI: 0.11-0.23)) than those men with a raised PSA but negative for cancer on biopsy (FIG. 22).

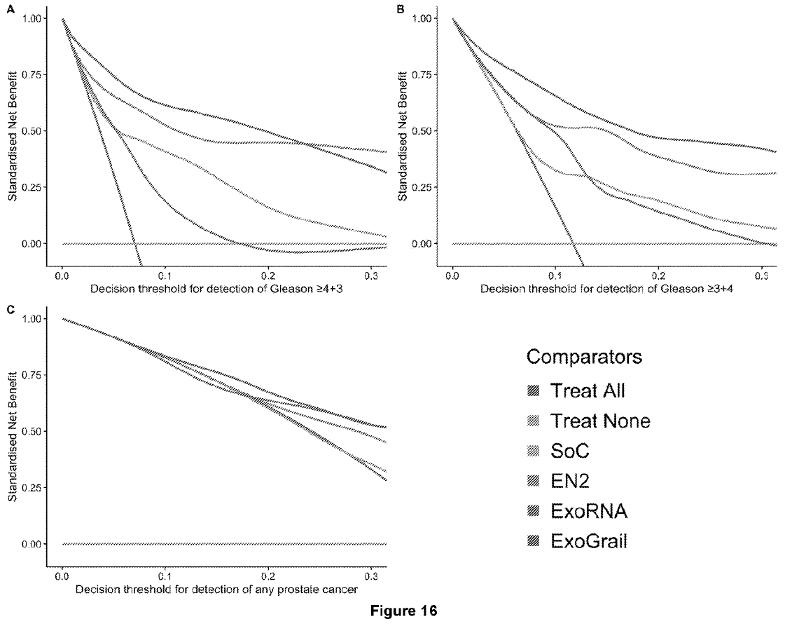

Decision curve analyses examined the net benefit of adopting ExoGrail in a population of patients suspected with prostate cancer and to have a PSA level suitable to trigger biopsy (≥4 ng/mL). The biopsy of men based upon their ExoGrail risk score consistently provided a net benefit over current standards of care across all decision thresholds examined and was the most consistent amongst all comparator models across a range of clinically relevant endpoints for biopsy (FIG. 16).

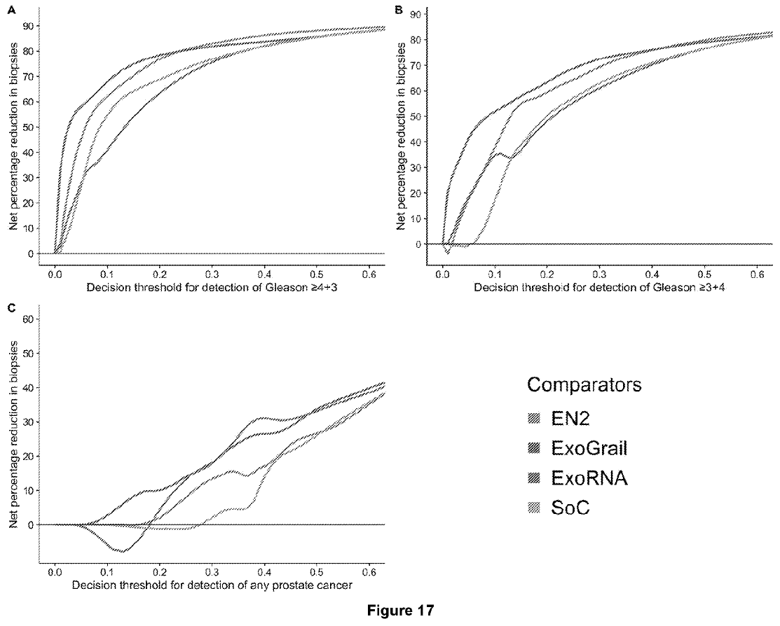

At a decision threshold of 0.25, ExoGrail could result in up to 69% fewer unnecessary biopsies of men presenting with a suspicion of prostate cancer, without missing substantial numbers of men with aggressive disease, whilst if Gleason ≥4+3 were considered the threshold of clinical significance, the same decision threshold of 0.25 could save 80% of men from receiving an unnecessary biopsy (FIG. 17).

### Example 7—Expression Analyses

### Gene Transcript Analysis

NanoString® expression analysis (167 probes, 164 genes, Table 7) was performed. 137 probes were selected based on previously proposed controls plus prostate cancer diagnostic and prognostic biomarkers within tissue and control probes. 30 additional probes were selected as overexpressed in prostate cancer samples when next generation sequence data generated from 20 urine EV RNA samples were analysed. Target gene sequences were provided to NanoString®, who designed the probes according to their protocols [71]. Data were adjusted relative to internal positive control probes as stated in NanoString®'s protocols.

TABLE 7
Genes initially identified for analysis with NanoString ®

| Gene | Full name | Accession number |
|---|---|---|
| AATF | apoptosis antagonizing transcription factor | NM_012138.3 |
| ABCB9 | ATP binding cassette subfamily B member 9 | NM_001243013.1 |
| ACTR5 | ARP5 actin-related protein 5 homolog | NM_024855.3 |
| AGR2 | anterior gradient 2, protein disulphide isomerase | NM_006408.2 |
| | family member | |
| ALAS1 | 5′-aminolevulinate synthase 1 | NM_000688.4 |
| AMACR | alpha-methylacyl-CoA racemase | NM_014324.4 |
| AMH | anti-Mullerian hormone | NM_000479.3 |
| ANKRD34B | ankyrin repeat domain 34B | NM_001004441.2 |
| ANPEP | alanyl aminopeptidase, membrane | NM_001150.1 |
| APOC1 | apolipoprotein C1 | NM_001645.3 |
| AR ex 9 | Androgen Receptor splice variant | ENST00000514029.1 |
| AR ex 4-8 | Androgen Receptor | NM_000044.2 |
| ARHGEF25 | Rho guanine nucleotide exchange factor 25 | NM_001111270.2 |
| AURKA | aurora kinase A | NM_003600.2 |
| Gene | Full name | Accession number |
| B2M | beta-2-microglobulin | NM_004048.2 |
| B4GALNT4 | beta-1,4-N-acetyl-galactosaminyltransferase 4 | NM_178537.4 |
| BRAF | B-Raf proto-oncogene, serine/threonine kinase | NM_004333.3 |
| BTG2 | BTG anti-proliferation factor 2 | NM_006763.2 |
| CACNA1D | calcium voltage-gated channel subunit alpha1 D | NM_000720.3 |
| CADPS | calcium dependent secretion activator | NM_183394.2 |
| CAMK2N2 | calcium/calmodulin dependent protein kinase II | NM_033259.2 |
| | inhibitor 2 | |
| CAMKK2 | calcium/calmodulin dependent protein kinase kinase 2 | NM_006549.3 |
| CASKIN1 | CASK interacting protein 1 | NM_020764.3 |
| CCDC88B | coiled-coil domain containing 88B | NM_032251.5 |
| CDC20 | cell division cycle 20 | NM_001255.2 |
| CDC37L1 | cell division cycle 37 like 1 | NM_017913.2 |
| CDKN3 | cyclin dependent kinase inhibitor 3 | NM_005192.3 |
| CERS1 | ceramide synthase 1 | NM_198207.2 |
| CKAP2L | cytoskeleton associated protein 2 like | NM_152515.3 |
| CLIC2 | chloride intracellular channel 2 | NM_001289.4 |
| CLU | clusterin | NM_203339.1 |
| COL10A1 | collagen type X alpha 1 chain | NM_000493.3 |
| COL9A2 | collagen type IX alpha 2 chain | NM_001852.3 |
| CP | ceruloplasmin | NM_000096.3 |
| MIATNB | MIAT neighbour | CTA_211A95.1 |
| DLX1 | distal-less homeobox 1 | NM_001038493.1 |
| DNAH5 | dynein axonemal heavy chain 5 | NM_001369.2 |
| DPP4 | dipeptidyl peptidase 4 | NM_001935.3 |
| ECI2 | enoyl-CoA delta isomerase 2 | NM_006117.2 |
| EIF2D | eukaryotic translation initiation factor 2D | NM_006893.2 |
| EN2 | engrailed homeobox 2 | NM_001427.3 |
| TMPRSS2/ERG | transmembrane protease, serine 2/ERG fusion | Fusion_0120.1 |
| | | EU432099.1 |
| ERG | ERG, ETS transcription factor | NM_001243428.1 |
| ERG ex 4-5 | ERG, ETS transcription factor | NM_004449.4 |
| ERG ex 6-7 | ERG, ETS transcription factor | NM_182918.3 |
| FDPS | farnesyl diphosphate synthase | NM_001135822.1 |
| FOLH1 | folate hydrolase 1 | NM_004476.1 |
| GABARAPL2 | GABA type A receptor associated protein like 2 | NM_007285.6 |
| GAPDH | glyceraldehyde-3-phosphate dehydrogenase | NM_002046.3 |
| GCNT1 | glucosaminyl (N-acetyl) transferase 1, core 2 | NM_001097633.1 |
| GDF15 | growth differentiation factor 15 | NM_004864.2 |
| GJB1 | gap junction protein beta 1 | NM_000166.5 |
| GOLM1 | golgi membrane protein 1 | NM_016548.3 |
| HIST1H1C | histone cluster 1 H1 family member c | NM_005319.3 |
| HIST1H1E | histone cluster 1 H1 family member e | NM_005321.2 |
| HIST1H2BF | histone cluster 1 H2B family member f | NM_003522.3 |
| HIST1H2BG | histone cluster 1 H2B family member g | NM_003518.3 |
| HIST3H2A | histone cluster 3 H2A | NM_033445.2 |
| HMBS | hydroxymethylbilane synthase | NM_000190.3 |
| HOXC4 | homeobox C4 | NM_014620.4 |
| HOXC6 | homeobox C6 | NM_153693.3 |
| HPN | hepsin | NM_182983.1 |
| HPRT1 | hypoxanthine phosphoribosyltransferase 1 | NM_000194.1 |
| IFT57 | intraflagellar transport 57 | NM_018010.2 |
| IGFBP3 | insulin like growth factor binding protein 3 | NM_000598.4 |
| IMPDH2 | inosine monophosphate dehydrogenase 2 | NM_000884.2 |
| ISX | intestine specific homeobox | NM_001008494.1 |
| ITGBL1 | integrin subunit beta like 1 | NM_004791.2 |
| ITPR1 | inositol 1,4,5-trisphosphate receptor type 1 | NM_001099952.1 |
| KLK2 | kallikrein related peptidase 2 | NM_005551.3 |
| KLK3 ex 1-2 | kallikrein related peptidase 3 | NM_001030048.1 |
| KLK3 ex 2-3 | kallikrein related peptidase 3 | NM_001648.2 |
| KLK4 | kallikrein related peptidase 4 | NM_004917.3 |
| LBH | limb bud and heart development | NM_030915.3 |
| POTEH-AS1 | POTEH antisense RNA 1 (POTEH-AS1), long | NR_110505.1 |
| | non-coding RNA. prostate-specific P712P mRNA | |
| MAK | male germ cell associated kinase | NM_005906.3 |
| MAPK8IP2 | mitogen-activated protein kinase 8 interacting protein | NM_012324.2 |
| | 2 | |
| MARCH5 | membrane associated ring-CH-type finger 5 | NM_017824.4 |
| MCM7 | minichromosome maintenance complex component 7 | NM_182776.1 |
| MCTP1 | multiple C2 and transmembrane domain containing 1 | NM_024717.4 |
| MDK | midkine (neurite growth-promoting factor 2) | NM_001012334.1 |
| MED4 | mediator complex subunit 4 | NM_001270629.1 |
| MEMO1 | mediator of cell motility 1 | NM_001137602.1 |
| MET | MET proto-oncogene, receptor tyrosine kinase | NM_001127500.1 |
| MEX3A | mex-3 RNA binding family member A | NM_001093725.1 |
| MFSD2A | major facilitator superfamily domain containing 2A | NM_032793.4 |
| MGAT5B | mannosyl (alpha-1,6-)-glycoprotein | NM_144677.2 |
| | beta-1,6-N-acetyl-glucosaminyltransferase, isozyme B | |
| MIR146A | microRNA 146a | ENST00000517927.1 |
| MIR4435-2HG | MIR4435-2 host gene | ENST00000409569b.1 |
| MKI67 | marker of proliferation Ki-67 | NM_002417.2 |
| MME | membrane metalloendopeptidase | NM_000902.2 |
| MMP11 | matrix metallopeptidase 11 | NM_005940.3 |
| MMP25 | matrix metallopeptidase 25 | NM_022468.4 |
| Gene | Full name | Accession number |
| MMP26 | matrix metallopeptidase 26 | NM_021801.3 |
| MNX1 | motor neuron and pancreas homeobox 1 | NM_005515.3 |
| MSMB | microseminoprotein beta | NM_002443.2 |
| MX11 | MAX interactor 1, dimerization protein | NM_001008541.1 |
| MYOF | myoferlin | NM_013451.3 |
| NAALADL2 | N-acetylated alpha-linked acidic dipeptidase like 2 | NM_207015.2 |
| NEAT1 | nuclear paraspeckle assembly transcript 1 | NR_028272.1 |
| | (non-protein coding) | |
| NKAIN1 | Na+/K+ transporting ATPase interacting 1 | NM_024522.2 |
| NLRP3 | NLR family pyrin domain containing 3 | NM_001079821.2 |
| OGT | O-linked N-acetylglucosamine (GIcNAc) transferase | NM_181672.1 |
| OR51E2 | olfactory receptor family 51 subfamily E member 2 | NM_030774.2 |
| PALM3 | paralemmin 3 | NM_001145028.1 |
| PCA3 | prostate cancer associated 3 (non-protein coding) | NR_015342.1 |
| PCSK6 | proprotein convertase subtilisin/kexin type 6 | NM_138320.1 |
| PDLIM5 | PDZ and LIM domain 5 | NR_046186.1 |
| PLPP1 | phospholipid phosphatase 1 | NM_176895.1 |
| PPFIA2 | PTPRF interacting protein alpha 2 | NM_003625.2 |
| PPP1R12B | protein phosphatase 1 regulatory subunit 12B | NM_001167857.1 |
| PSTPIP1 | proline-serine-threonine phosphatase interacting | XM_006720737.1 |
| | protein 1 | |
| PTN | pleiotrophin | NM_002825.5 |
| PTPRC | protein tyrosine phosphatase, receptor type C | NM_080923.2 |
| PVT1 | Pvt1 oncogene (non-protein coding) | NR_003367.2 |
| RAB17 | RAB17, member RAS oncogene family | NR_033308.1 |
| RIOK3 | RIO kinase 3 | NM_003831.3 |
| RNF157 | ring finger protein 157 | NM_052916.2 |
| MRPL46 | mitochondrial ribosomal protein L46 | ENST00000561140.1 |
| RPL18A | ribosomal protein L18a | NM_000980.3 |
| RPL23AP53 | ribosomal protein L23a pseudogene 53 | NR_003572.2 |
| RPLP2 | ribosomal protein lateral stalk subunit P2 | NM_001004.3 |
| RPS10 | ribosomal protein S10 | NM_001014.3 |
| RPS11 | ribosomal protein S11 | NM_001015.3 |
| SACM1L | SAC1 suppressor of actin mutations 1-like (yeast) | NM_014016.3 |
| SCHLAP1 | SWI/SNF complex antagonist associated with | NR_104320.1 |
| | prostate cancer 1 (non-protein coding) | |
| SEC61A1 | Sec61 translocon alpha 1 subunit | NM_013336.3 |
| SERPINB5 | serpin family B member 5 | NM_002639.4 |
| SFRP4 | secreted frizzled related protein 4 | NM_003014.2 |
| SIM2 | single-minded family bHLH transcription factor 2 | NM_005069.3 |
| SIM2 | single-minded family bHLH transcription factor 2 | NM_009586.3 |
| SIRT1 | sirtuin 1 | NM_012238.4 |
| SLC12A1 | solute carrier family 12 member 1 | NM_000338.2 |
| SLC43A1 | solute carrier family 43 member 1 | NM_003627.5 |
| SLC4A1 | solute carrier family 4 member 1 | NM_000342.3 |
| SMAP1 | small ArfGAP 1 | NM_021940.3 |
| SMIM1 | small integral membrane protein 1 (Vel blood group) | ENST00000444870.1 |
| SNCA | synuclein alpha | NM_007308.2 |
| SNORA20 | Small nucleolar RNA SNORA20 | NR_002960.1 |
| SPINK1 | serine peptidase inhibitor, Kazal type 1 | NM_003122.2 |
| SPON2 | spondin 2 | NM_012445.1 |
| SRSF3 | serine and arginine rich splicing factor 3 | NM_003017.4 |
| SSPO | SCO-spondin | NM_198455.2 |
| SSTR1 | somatostatin receptor 1 | NM_001049.2 |
| ST6GALNAC1 | ST6 N-acetylgalactosaminide | ENST00000592042.1 |
| | alpha-2,6-sialyltransferase 1 | |
| STEAP2 | STEAP2 metalloreductase | NM_152999.2 |
| STEAP4 | STEAP4 metalloreductase | NM_024636.2 |
| STOM | stomatin | NM_004099.5 |
| SULF2 | sulfatase 2 | NM_001161841.1 |
| SULT1A1 | sulfotransferase family 1A member 1 | NM_177534.2 |
| SYNM | synemin | NM_015286.4 |
| TBP | TATA-box binding protein | NM_001172085.1 |
| TDRD1 | Tudor domain containing 1 | NM_198795.1 |
| TERF2IP | TERF2 interacting protein | NM_018975.3 |
| TERT | telomerase reverse transcriptase | NM_198253.1 |
| TFDP1 | transcription factor Dp-1 | NM_007111.4 |
| TIMP4 | TIMP metallopeptidase inhibitor 4 | NM_003256.2 |
| TMCC2 | transmembrane and coiled-coil domain family 2 | NM_014858.3 |
| TMEM45B | transmembrane protein 45B | NM_138788.3 |
| TMEM47 | transmembrane protein 47 | NM_031442.3 |
| TMEM86A | transmembrane protein 86A | NM_153347.1 |
| TRPM4 | transient receptor potential cation channel subfamily | NM_001195227.1 |
| | M member 4 | |
| TWIST1 | twist family bHLH transcription factor 1 | NM_000474.3 |
| UPK2 | uroplakin 2 | NM_006760.3 |
| VAX2 | ventral anterior homeobox 2 | NM_012476.2 |
| VPS13A | vacuolar protein sorting 13 homolog A | NM_033305.2 |
| ZNF577 | zinc finger protein 577 | NM_032679.2 |

TABLE 8
Genes of interest and associated capture probes

| | Gene | | | |
|---|---|---|---|---|
| Official | name | Accession | Capture probe | Reporter probe |
| symbol | Long | number | sequence | sequence |
| AATF | apoptosis | NM_012138.3 | TCATCATCTTCACTAGAA | CTCTTTGCAGGGACCCTTC |
| | antagonizing | (Accessed 5th | ATCTCCTCACTTCCCGCA | TTCGTTGCTGCTTCTTCTC |
| | transcription | Sep. 2019) | TTGGGCTTTGTCCC | TTCTACCAGC (SEQ ID |
| | factor | | (SEQ ID NO: 1) | NO: 2) |
| ABCB9 | ATP binding | NM_001243013.1 | GGGCCCCAGCGCACTGTT | ACGAAGAGGCACACGAGGG |
| | cassette | (Accessed 5th | CTTGGCCACACCAATGGT | TGATGACCAGCCACGAGGC |
| | subfamily B | Sep. 2019) | GG (SEQ ID NO: 3) | CCGCAGCCGCCG (SEQ |
| | member 9 | | | ID NO: 4) |
| ACTR5 | ARP5 | NM_024855.3 | CAAGGCATGGCGTGCAGG | GGCAGGTACATCTAGCACA |
| | actin-related | (Accessed 5th | GCAGTCTCTCTGGAGGG | ATCACAGTCCTGTCACACT |
| | protein 5 | Sep. 2019) | (SEQ ID NO: 5) | GCCAACGTGGCC (SEQ |
| | homolog | | | ID NO: 6) |
| AGR2 | anterior | NM_006408.2 | TGCCTCATCAACACGTCA | TGCCACAGCCTTTCACGTT |
| | gradient 2, | (Accessed 5th | CCACCCTTTGCTCTTCTT | TCCTAAACCCTAGTAACCT |
| | protein | Sep. 2019) | CCAATTAGTCACAT | CTGATCTCCATC (SEQ |
| | disulphide | | (SEQ ID NO: 7) | ID NO: 8) |
| | isomerase | | | |
| | family | | | |
| | member | | | |
| ALAS1 | 5′-aminolevulinate | NM_000688.4 | AGTGTTCCAGAAATGATG | GAGAACTCGTGCTGGCGAT |
| | synthase 1 | (Accessed 5th | TCCATTTTTGGCATGACT | GTACCCTCCAACACAACCA |
| | | Sep. 2019) | CCATCCCGATCCCC | AAGGCTTTGCCA (SEQ |
| | | | (SEQ ID NO: 9) | ID NO: 10) |
| AMACR | alpha-methylacyl- | NM_014324.4 | TGGAATCTACCCCTTCCT | CAACATCCATTCTCTACTC |
| | CoA | (Accessed 5th | CACATGCCTTTAGGAAGT | CCTCTACTCTGATGGCACC |
| | racemase | Sep. 2019) | TGAGTCCAGGGAAG | CGGATTAGATTG (SEQ |
| | | | (SEQ ID NO: 11) | ID NO: 12) |
| AMH | anti-Mullerian | NM_000479.3 | TTGGCCTGGTAGGTCTCG | CGGACTGAGGCCAGCCGCA |
| | hormone | (Accessed 5th | GGGATGAGTACGGAGCG | CACGCCCTGGCAATTG |
| | | Sep. 2019) | (SEQ ID NO: 13) | (SEQ ID NO: 14) |
| ANKRD34B | ankyrin | NM_001004441.2 | TTTATAGGATAGTTCTTC | ATGCTTTGGTGCCTAGTGA |
| | repeat | (Accessed 5th | CTCTGGTGTAATATCCTG | TGAACCGCTTGGAAAGTGC |
| | domain 34B | Sep. 2019) | GAGCTCCTCTTGCA | CAGCCCATTGGT (SEQ |
| | | | (SEQ ID NO: 15) | ID NO: 16) |
| ANPEP | alanyl | NM_001150.1 | GTAATGCTGATGATGGTA | AGTTGCTCTGGACAAAGTC |
| | aminopeptidase, | (Accessed 5th | GAGGTGGCGTCCTGCTTC | CCAGACCAGACCTTGCCCA |
| | membrane | Sep. 2019) | CGGATTAAGTC (SEQ | ATGACGTTGTTG (SEQ |
| | | | ID NO: 17) | ID NO: 18) |
| APOC1 | apolipoprotein | NM_001645.3 | CGGAGGGGCACTCTGAAT | CAGAACCACCACCAGGACC |
| | C1 | (Accessed 5th | CCTTGCTGGAGGGCTTGG | GGGAGCGACAGGAAGAGCC |
| | | Sep. 2019) | TTGGGAGGTC (SEQ ID | TCATGGCGAGGC (SEQ |
| | | | NO: 19) | ID NO: 20) |
| AR ex 9 | Androgen | ENST0000051402 | TTTGAAGAGAGGGGTTGG | CAGTAAGGCTAGATGTAAG |
| | Receptor | 9.1 (Accessed 5th | CTGGCTTCTTCTCCTGGA | AGGGAAAGTCGGACTGTAG |
| | splice | Sep. 2019) | GAAGCAGAAATCTG | TCTCTCAGTGTG (SEQ |
| | variant | | (SEQ ID NO: 21) | ID NO: 22) |
| AR ex 4-8 | Androgen | NM_000044.2 | GACTTGTGCATGCGGTAC | CAAACTCTTGAGAGAGGTG |
| | Receptor | (Accessed 5th | TCATTGAAAACCAGATCA | CCTCATTCGGACACACTGG |
| | | Sep. 2019) | GGGGCGAAGTAGAG | CTGTACATCCGG (SEQ |
| | | | (SEQ ID NO: 23) | ID NO: 24) |
| ARHGEF25 | Rho guanine | NM_001111270.2 | CAGCGCTTGGGCACAAAG | CTCAAATCCCCGCAATCTC |
| | nucleotide | (Accessed 5th | CACATGACCTCCACAGCT | CCCAGCGTCATCATATCGT |
| | exchange | Sep. 2019) | TG (SEQ ID NO: 25) | TG (SEQ ID NO: 26) |
| | factor 25 | | | |
| AURKA | aurora | NM_003600.2 | AAGGAAATTGCTGAGTCA | ACACAAGACCCGCTGAGCC |
| | kinase A | (Accessed 5th | CGAGAACACGTTTTGGAC | TGGCCACTATTTACAGGTA |
| | | Sep. 2019) | CTCCAACTGGAGCT | ATGGATTCTGAC (SEQ |
| | | | (SEQ ID NO: 27) | ID NO: 28) |
| B2M | beta-2-micro | NM_004048.2 | CACGGAGCGAGACATCTC | CAGGCCAGAAAGAGAGAGT |
| | globulin | (Accessed 5th | GGCCCGAATGCTGTCAGC | AGCGCGAGCACAGCTAAGG |
| | | Sep. 2019) | TT (SEQ ID NO: 29) | C (SEQ ID NO: 30) |
| B4GALNT4 | beta-1,4-N-acetyl- | NM_178537.4 | TCCCTCGCCGGGTGGATG | CAGAACTCCGAGTTGTCGT |
| | galactosaminyl- | (Accessed 5th | AAACCAAAAATACGGAGT | CTGAGGCCACAGAAAACTG |
| | transferase 4 | Sep. 2019) | CCATAGTTCTTCCA | GACGTCTCCG (SEQ ID |
| | | | (SEQ ID NO: 31) | NO: 32) |
| BRAF | B-Raf | NM_004333.3 | AGTGCTTTCTTTAGACTG | CCTGAATTCTGTAAACAGC |
| | proto-oncogene, | (Accessed 5th | TCTCGGACTGTAACTCCA | ACAGCACTCTGGGATTAGA |
| | serine/ | Sep. 2019) | CACCTTGCAGGTAC | CCTCTCATCATC (SEQ |
| | threonine | | (SEQ ID NO: 33) | ID NO: 34) |
| | kinase | | | |
| BTG2 | BTG | NM_006763.2 | CAAGGAATACATGCAAGG | ACAAGAATACCAAGTAGTC |
| | anti-proliferation | (Accessed 5th | CTGACTAGCCAGCCATCA | TTGCAGAACATGGGGCACT |
| | factor | Sep. 2019) | TCCCAAGGAGAG (SEQ | CTCCCATTCAGC (SEQ |
| | 2 | | ID NO: 35) | ID NO: 36) |
| CACNA1D | calcium | NM_000720.3 | GTACTTCTGGGCTTTACT | GTTGCTGGAGGGGTGGCCC |
| | voltage-gated | (Accessed 5th | TGAATCTAGGCCGGCAAC | ACGACCGGGTCGAGTGACT |
| | channel | Sep. 2019) | TGCCATGATCTGTT | CGGTGA (SEQ ID NO: |
| | subunit | | (SEQ ID NO: 37) | 38) |
| | alpha1 D | | | |
| CADPS | calcium | NM_183394.2 | TTGAGGCTTATCCATTCG | TTCCAGACATTCTTACCGA |
| | dependent | (Accessed 5th | GACAGCAAGTTTGATTTT | TGGCCCATAAATACCCAGA |
| | secretion | Sep. 2019) | GAGATCTTGGTCGG | ATGCTTCATGTT (SEQ |
| | activator | | (SEQ ID NO: 39) | ID NO: 40) |
| CAMK2N2 | calcium/ | NM_033259.2 | AAATACAAATGTGCTGAG | GGGAGGGCAGGAACCATGA |
| | calmodulin | (Accessed 5th | GAAGTCCCTTAGAAAGAG | GCAGAGCCAGTAAACAAAG |
| | dependent | Sep. 2019) | GCTGAGGCTGGGGT | AGTCGGATATAA (SEQ |
| | protein | | (SEQ ID NO: 41) | ID NO: 42) |
| | kinase Il | | | |
| | inhibitor 2 | | | |
| CAMKK2 | calcium/ | NM_006549.3 | GGTGGATGATCTTCTGGT | CTTGATGTGCCCATCTTCT |
| | calmodulin | (Accessed 5th | AGTGTAAGTACTCGATGC | CCGACCAGGAGGTTGGAAG |
| | dependent | Sep. 2019) | CTTTGATCAGATCC | GTTTGATGTCAC (SEQ |
| | protein | | (SEQ ID NO: 43) | ID NO: 44) |
| | kinase | | | |
| | kinase 2 | | | |
| CASKIN1 | CASK | NM_020764.3 | ACCTTGTAGTACTGGGCC | AGGTGATGTCGGTGATGAA |
| | interacting | (Accessed 5th | AGGCCGATCATGGACAG | ATCAATGTTCTCGTAGCCA |
| | protein 1 | Sep. 2019) | (SEQ ID NO: 45) | TTGTCCACCAAC (SEQ |
| | | | | ID NO: 46) |
| CCDC88B | coiled-coil | NM_032251.5 | TCCACCGCTTCTTCTGAG | TGACGCTCCCAACAGTAGC |
| | domain | (Accessed 5th | AGAGGGTCAAATCCCAAT | CGAAGAACGCCTTCCAGCT |
| | containing | Sep. 2019) | GTCTG (SEQ ID NO: | GC (SEQ ID NO: 48) |
| | 88B | | 47) | |
| CDC20 | cell division | NM_001255.2 | CCTCTACATCAAAACCGT | ACCCTCTGGCGCATTTTGT |
| | cycle 20 | (Accessed 5th | TCAGGTTCAAAGCCCAGG | GGTTTTCCACTGAGCCGAA |
| | | Sep. 2019) | CTTTCTGATGTTCC | GGATCTTGGCTT (SEQ |
| | | | (SEQ ID NO: 49) | ID NO: 50) |
| CDC37L1 | cell division | NM_017913.2 | TCATCTTCTTTATGTACC | GGCCTCAGCAGTCTTAACC |
| | cycle 37 like | (Accessed 5th | ACCGAGTTTAAGCTGCAG | AAATTATACAGTGTCCATC |
| | 1 | Sep. 2019) | AGAGCTGTACTGAT | ATTTTGGGTTCA (SEQ |
| | | | (SEQ ID NO: 51) | ID NO: 52) |
| CDKN3 | cyclin | NM_005192.3 | AGACAAGATCTCCCAAGT | CTCTGGTGATATTGTGTCA |
| | dependent | (Accessed 5th | CCTCCATAGCAGTGTATT | GACAGGTATAGTAGGAGAC |
| | kinase | Sep. 2019) | AAGGTTTTTCGGTA | AAGCAGCTACA (SEQ ID |
| | inhibitor 3 | | (SEQ ID NO: 53) | NO: 54) |
| CERS1 | ceramide | NM_198207.2 | GCATCTCGCACCTCCCGT | CTGCCTGGCTACAGCCCCG |
| | synthase 1 | (Accessed 5th | TCCAAAAAACGTCACGGA | GATGTGTTAAATGTCT |
| | | Sep. 2019) | GCTCTGAG (SEQ ID | (SEQ ID NO: 56) |
| | | | NO: 55) | |
| CKAP2L | cytoskeleton | NM_152515.3 | TGAGGTATACAAACTTGG | AATTAGGCCTCTGGCTTAT |
| | associated | (Accessed 5th | CTGGACTTCTGATCTTGC | GGCTTTTGACTTTTGCAGT |
| | protein 2 | Sep. 2019) | TTGATGTTTGGATG | ACACATGATGTC (SEQ |
| | like | | (SEQ ID NO: 57) | ID NO: 58) |
| CLIC2 | chloride | NM_001289.4 | CCAGTCTCTTCTCTCAAG | TGCTTTAAGAAGACCGTCT |
| | intracellular | (Accessed 5th | AGGTGTGACGCAGAAAAT | AGCTTGTAGTGGACTGAGT |
| | channel 2 | Sep. 2019) | TCTAGATGCTTAAG | CAGACCTGGAG (SEQ ID |
| | | | (SEQ ID NO: 59) | NO: 60) |
| CLU | clusterin | NM_203339.1 | GCCTGTGGTCCAGGGAAA | AGCGTAGGGTACTGCAGCC |
| | | (Accessed 5th | GGTATGAAGATCATATAA | CAGCTATGGTTCAGACTAA |
| | | Sep. 2019) | ACCGGCGGTGGACA | AAGCCGAGAAAC (SEQ |
| | | | (SEQ ID NO: 61) | ID NO: 62) |
| COL10A1 | collagen | NM_000493.3 | CCTGTGGGCATTTGGTAT | TGTAGGGAATGAAGAACTG |
| | type X alpha | (Accessed 5th | CGTTCAGCGTAAAACACT | TGTCTTGGTGTTGGGTAGT |
| | 1 chain | Sep. 2019) | CCATGAACCAAGTT | GGGCCTTTTATG (SEQ |
| | | | (SEQ ID NO: 63) | ID NO: 64) |
| COL9A2 | collagen | NM_001852.3 | CGATAGCGCCCACCATGC | CCTAGGACCTTCCTCACCC |
| | type IX alpha | (Accessed 5th | CTTTATATCCATGAGGGC | GGTGGCCCAGTGGCAC |
| | 2 chain | Sep. 2019) | CCGTCTCTCCCTTG | (SEQ ID NO: 66) |
| | | | (SEQ ID NO: 65) | |
| CP | ceruloplasmin | NM_000096.3 | CTTGCCCGTGAAAGAAAG | AGCAGGAAAGAGGTTGATT |
| | | (Accessed 5th | CTGCGTGCACATCAACTT | GTGTCAATACGGTAGTTCT |
| | | Sep. 2019) | CATTACCCATACCA | TGTTAGTCAGTG (SEQ |
| | | | (SEQ ID NO: 67) | ID NO: 68) |
| MIATNB | MIAT | CTA_211A95.1 | CTGGAGGTATCCAAGAGT | GAAGAGCCCAAACCTGCCT |
| | neighbour | (Accessed 5th | CTGCCGAGGGACTTCAAG | GGCTTCAAAACAGGTGGTG |
| | | Sep. 2019) | TATTCAGGAAGGGG | AGCTCCCCATTG (SEQ |
| | | | (SEQ ID NO: 69) | ID NO: 70) |
| DLX1 | distal-less | NM_001038493.1 | CAGCCTCAGGCGAAGTCC | CGTTTGAACAGTGCGTTCC |
| | homeobox 1 | (Accessed 5th | ATTTCTCAATAAATAAAA | TTGCGCCCAGCAGAACCCT |
| | | Sep. 2019) | CCCCCTCCCTCCAA | GAATTGGCAAA (SEQ ID |
| | | | (SEQ ID NO: 71) | NO: 72) |
| DNAH5 | dynein | NM_001369.2 | GGCGGAACGCATCATGTA | CTGAAGGAGTGTAATGGGA |
| | axonemal | (Accessed 5th | CAAGCTCAGTTTCTATGA | AACTGCTTATGAGCCTCGG |
| | heavy chain | Sep. 2019) | TTATGTCCATCAGC | TGGTCATCCAGA (SEQ |
| | 5 | | (SEQ ID NO: 73) | ID NO: 74) |
| DPP4 | dipeptidyl | NM_001935.3 | AAATCCACTCCAACATCG | CTGCTAGCTATTCCATGGT |
| | peptidase 4 | (Accessed 5th | ACCAGGGCTTTGGAGATC | CTTCATCAGTATACCACAT |
| | | Sep. 2019) | TGAGCTGACTGCTG | TGCCTGG (SEQ ID NO: |
| | | | (SEQ ID NO: 75) | 76) |
| ECI2 | enoyl-CoA | NM_006117.2 | GAAAACTTCAGTAACAAG | CAAATGCCTTCAGCCTGGT |
| | delta | (Accessed 5th | TCCTTGAGCACATGCCTC | CCAGACTTCTTTCTGAAAA |
| | isomerase 2 | Sep. 2019) | TCCCGCTGTTAACT | GTGCTATCAGG (SEQ ID |
| | | | (SEQ ID NO: 77) | NO: 78) |
| EIF2D | eukaryotic | NM_006893.2 | GCTCTTGTCCGGGAAGGG | TTGTGCTAGGGTGATGTCA |
| | translation | (Accessed 5th | TCACTTGATAGGCAGGCT | ATTGGACAGATTCTCCCTT |
| | initiation | Sep. 2019) | GTAATTTTTCCAAA | TCTTCACAATGG (SEQ |
| | factor 2D | | (SEQ ID NO: 79) | ID NO: 80) |
| EN2 | engrailed | NM_001427.3 | AAGGTAGCCACATGTTTC | CTTTCTTCCTTCTTCTAGA |
| | homeobox 2 | (Accessed 5th | AGAACTGTGGACTCAAAC | TCCTGGAGGATTCTGAGTT |
| | | Sep. 2019) | ACGCCTGGTGTGTG | CTTTTGAAAGAC (SEQ |
| | | | (SEQ ID NO: 81) | ID NO: 82) |
| TMPRSS2/ERG | transmembrane | Fusion_0120.1 | CTGCCGCGCTCCAGGCGG | TAGGCACACTCAAACAACG |
| | protease, | (Accessed 5th | CGCTCCCCGCCCCTCGC | ACTGGTCCTCACTCACAAC |
| | serine 2/ERG | Sep. 2019) | (SEQ ID NO: 83) | TGATAAGGCTTC (SEQ |
| | fusion | | | ID NO: 84) |
| ERG | ERG, ETS | NM_001243428.1 | CCATCTTTTTTCTCTGTG | CCATCTACCAGCTGTTCAG |
| | transcription | (Accessed 5th | AGTCATTTGTCTTGCTTT | AACCTGACGGCTTTAGTTG |
| | factor | Sep. 2019) | TGGTCAACACGGCT | CCCTTGGTTCTG (SEQ |
| | | | (SEQ ID NO: 85) | ID NO: 86) |
| ERG ex 4-5 | ERG, ETS | NM_004449.4 | TGAGCCATTCACCTGGCT | CCACCATCTTCCCGCCTTT |
| | transcription | (Accessed 5th | AGGGTTACATTCCATTTT | GGCCACACTGCATTCATCA |
| | factor | Sep. 2019) | GATGGTGACCCTGG | GGAGAGTTCCT (SEQ ID |
| | | | (SEQ ID NO: 87) | NO: 88) |
| ERG ex 6-7 | ERG, ETS | NM_182918.3 | ACATCATCTGAAGTCAAA | CTGTGTTTCTAGCATGCAT |
| | transcription | (Accessed 5th | TGTGGAAGAGGAGTCTCT | TAACCGTGGAGAGTTTTGT |
| | factor | Sep. 2019) | CTGAGGTAGTGGAG | AAGGCTTTATCA (SEQ |
| | | | (SEQ ID NO: 89) | ID NO: 90) |
| FDPS | farnesyl | NM_001135822.1 | CATCCTGTTTCCTTGGCT | CCAGCCCACAGTCCAGGCC |
| | diphosphate | (Accessed 5th | CCACCAGCTCCCGGAATG | CGCTGGAGACTATCAG |
| | synthase | Sep. 2019) | CTACTAC (SEQ ID | (SEQ ID NO: 92) |
| | | | NO: 91) | |
| FOLH1 | folate | NM_004476.1 | TGAAAGGTGGTACAATAT | GTTAACATACACTAGATCG |
| | hydrolase 1 | (Accessed 5th | CCGAAACATTTTCATATC | CCCTCTGGCATTCCTTGAG |
| | | Sep. 2019) | CTGGAGGAGGTGGT | GAGAGAAAGCAC (SEQ |
| | | | (SEQ ID NO: 93) | ID NO: 94) |
| GABARAPL2 | GABA type A | NM_007285.6 | GGGACTGTCTTATCCACA | CTTCATCTTTTTCCTTCTC |
| | receptor | (Accessed 5th | AACAGGAAGATCGCCTTT | GTAAAGCTGTCCCATAGTT |
| | associated | Sep. 2019) | TCAGAAGGAAGCTG | AGGCTGGACTGT (SEQ |
| | protein like | | (SEQ ID NO: 95) | ID NO: 96) |
| | 2 | | | |
| GAPDH | glyceraldehyde- | NM_002046.3 | AAGTGGTCGTTGAGGGCA | CCCTGTTGCTGTAGCCAAA |
| | 3-phosphate | (Accessed 5th | ATGCCAGCCCCAGCGTCA | TTCGTTGTCATACCAGGAA |
| | dehydrogenase | Sep. 2019) | AAG (SEQ ID NO: | ATGAGCTTGACA (SEQ |
| | | | 97) | ID NO: 98) |
| GCNT1 | glucosaminyl | NM_001097633.1 | TTTCAAACAATAATCAGG | GTATTTGGTGGGATAAGAA |
| | (N-acetyl) | (Accessed 5th | GATTTCCTTTGTGAAGGG | AAAAGTCTCCTTCGCAGCA |
| | transferase | Sep. 2019) | CAGTCTTCTATGCT | ACGTCCTCAGCA (SEQ |
| | 1, core 2 | | (SEQ ID NO: 99) | ID NO: 100) |
| GDF15 | growth | NM_004864.2 | CCTGGTTAGCAGGTCCTC | GTGTTCGAATCTTCCCAGC |
| | differentiation | (Accessed 5th | GTAGCGTTTCCGCAACTC | TCTGGTTGGCCCGCAG |
| | factor 15 | Sep. 2019) | (SEQ ID NO: 101) | (SEQ ID NO: 102) |
| GJB1 | gap junction | NM_000166.5 | TGAAGATGAAGATGACCG | TTTCTCATCACCCCACACA |
| | protein beta | (Accessed 5th | AGAGCCATACTCGGCCAA | CTCTCTGCAGCCACCACCA |
| | 1 | Sep. 2019) | TGGCAGTAGAATGC | GCACCATGATTC (SEQ |
| | | | (SEQ ID NO: 103) | ID NO: 104) |
| GOLM1 | golgi | NM_016548.3 | GGATGAGCCTCTCACCTG | TAATTCCTCTGCAGGGTCT |
| | membrane | (Accessed 5th | TGGTGATGTTATTCACCA | TTAACTGGTCTTGCAGCAC |
| | protein 1 | Sep. 2019) | AAACCGC (SEQ ID | TC (SEQ ID NO: 106) |
| | | | NO: 105) | |
| HIST1H1C | histone | NM_005319.3 | CTTGGCTGCCCCAACTGG | TTCGGAGTTGCGCCGCCAG |
| | cluster 1 H1 | (Accessed 5th | CTTCTTAGGTTTGGTTCC | CCGCCTTCTTGGGCTT |
| | family | Sep. 2019) | GCCCGCCTTTTTAA | (SEQ ID NO: 108) |
| | member c | | (SEQ ID NO: 107) | |
| HIST1H1E | histone | NM_005321.2 | GCGCTCCTTGGAGGCGGC | CTGCCAGCGCTTTCTTGAG |
| | cluster 1 H1 | (Accessed 5th | AACAGCTTTAGTAATGAG | AGCGGCCAAAGATACGCCG |
| | family | Sep. 2019) | CTCGG (SEQ ID NO: | CT (SEQ ID NO: 110) |
| | member e | | 109) | |
| HIST1H2BF | histone | NM_003522.3 | CTTGGTGACGGCCTTGGT | AGCCTTTGGGATTGGGTAT |
| | cluster 1 | (Accessed 5th | GCCCTCTGACACGGCGTG | GAAGACGTTAGAATTACTT |
| | H2B family | Sep. 2019) | (SEQ ID NO: 111) | AGAGCTGGTGTA (SEQ |
| | member f | | | ID NO: 112) |
| HIST1H2BG | histone | NM_003518.3 | TATACTTGGTGACAGCCT | AAGAGCCTTTGAGTTTTAA |
| | cluster 1 | (Accessed 5th | TGGTACCTTCGGACACTG | AGCACCTAAGCACACATTT |
| | H2B family | Sep. 2019) | CGTGCTTGG (SEQ ID | ACTTGGAGCTTG (SEQ |
| | member g | | NO: 113) | ID NO: 114) |
| HIST3H2A | histone | NM_033445.2 | CGGAGCAACCGGTGCACG | CGCCGGCGCCCACGCGCTC |
| | cluster 3 | (Accessed 5th | CGGCCCACGGGGAACTG | CGAATAGTTGCCCTTG |
| | H2A | Sep. 2019) | (SEQ ID NO: 115) | (SEQ ID NO: 116) |
| HMBS | hydroxymet | NM_000190.3 | GCTGGGCAGGGACATGGA | AGTGATGCCTACCAACTGT |
| | hylbilane | (Accessed 5th | TGGTAGCCTGCATGGTC | GGGTCATCCTCAGGGCCAT |
| | synthase | Sep. 2019) | (SEQ ID NO: 117) | CTTCAT (SEQ ID NO: |
| | | | | 118) |
| HOXC4 | homeobox | NM_014620.4 | TGAATTTTTTTCATCCAT | CGCTTGGGTTCCCCTCCGT |
| | C4 | (Accessed 5th | GGGTAGACTATGGGTTGC | TATAATTGGGGTTCACCGT |
| | | Sep. 2019) | TTGCTGGCGGCG (SEQ | GCTAACG (SEQ ID NO: |
| | | | ID NO: 119) | 120) |
| HOXC6 | homeobox | NM_153693.3 | GGTCGAGAAATGCCTCAC | GAATAAAAGGGAGTCGAGT |
| | C6 | (Accessed 5th | TGGATCATAGGCGGTGGA | AGATCCGGTTCTGGGCAAC |
| | | Sep. 2019) | ATTGAGGGCGACGT | GGCCGCTCCATA (SEQ |
| | | | (SEQ ID NO: 121) | ID NO: 122) |
| HPN | hepsin | NM_182983.1 | CCGAGAGATGCTGTCCTC | CCAACTCACAATGCCACAC |
| | | (Accessed 5th | ACACACAAAGGGACCACC | AGCCGCCAACGTGGCGT |
| | | Sep. 2019) | GCTG (SEQ ID NO: | (SEQ ID NO: 124) |
| | | | 123) | |
| HPRT1 | hypoxanthine | NM_000194.1 | TGAGCACACAGAGGGCTA | CAGTGCTTTGATGTAATCC |
| | phosphoribosyl- | (Accessed 5th | CAATGTGATGGCCTCCCA | AGCAGGTCAGCAAAGAATT |
| | transferase | Sep. 2019) | TCTCCTTCATCACA | TATAGCCCCCCT (SEQ |
| | 1 | | (SEQ ID NO: 125) | ID NO: 126) |
| IFT57 | intraflagellar | NM_018010.2 | AATCGTGACTTTCAGTTG | TGCTGGTGCATTTGGTCAA |
| | transport 57 | (Accessed 5th | CGGTAGTACACGTTCCAC | CATGGATTCTCCAATCCTT |
| | | Sep. 2019) | TTCTAGGCTCCATT | ATTGTCAGTCCT (SEQ |
| | | | (SEQ ID NO: 127) | ID NO: 128) |
| IGFBP3 | insulin like | NM_000598.4 | CGGGCGCATGAAGTCTGG | TGGTCGGCCGCTTCGACCA |
| | growth | (Accessed 5th | GTGCTGTGCTCGAGTCTC | ACATGTGGTGAGCATTCCA |
| | factor | Sep. 2019) | TGAATATTTTGATA | (SEQ ID NO: 130) |
| | binding | | (SEQ ID NO: 129) | |
| | protein 3 | | | |
| IMPDH2 | inosine | NM_000884.2 | TCTTTGAGAAAATCAATG | TCCCTCTTTGTCATTATCT |
| | monophosphate | (Accessed 5th | TCCCTGGAGGAGATGATG | CTTCCAAGAAACAGTCATG |
| | dehydrogenase | Sep. 2019) | CCCACCAAGCGGCT | TTCCTCC (SEQ ID NO: |
| | 2 | | (SEQ ID NO: 131) | 132) |
| ISX | intestine | NM_001008494.1 | ATCTGGCATTTTTAAGAT | TGCTAGAGACCTGGTGTTG |
| | specific | (Accessed 5th | GGCAAAGCACTTTTGCAT | ATATCCACATTCATAGGCT |
| | homeobox | Sep. 2019) | CCTGTGGGCTGTTG | CTGAGTG (SEQ ID NO: |
| | | | (SEQ ID NO: 133) | 134) |
| ITGBL1 | integrin | NM_004791.2 | AGACCACACCATCGAGGT | TCCTCTCTCACAAACACAG |
| | subunit beta | (Accessed 5th | CTTCACAGCGGCGATCAT | CGACCACAGGAACATGTGC |
| | like 1 | Sep. 2019) | CACACTCACAAGTC | CGTGGCCTCCAC (SEQ |
| | | | (SEQ ID NO: 135) | ID NO: 136) |
| ITPR1 | inositol | NM_001099952.1 | GACAATCTCTATCTGCGC | CATATGCTGGGCACGGGAA |
| | 1,4,5- | (Accessed 5th | CGTGTGCTTGGCATAAAA | AGACTATCTGTTCCATTGT |
| | trisphosphate | Sep. 2019) | CTCCAGGGC (SEQ ID | TCGGTCTAATCT (SEQ |
| | receptor | | NO: 137) | ID NO: 138) |
| | type 1 | | | |
| KLK2 | kallikrein | NM_005551.3 | CTTGGACACTAAGGATCA | GTCAATTATTCAAGTACTC |
| | related | (Accessed 5th | GGTGAGCTTCCTCAGTTG | CATACTCGTCCTACAGACC |
| | peptidase 2 | Sep. 2019) | GAATTACTTTGTAC | CCCAGTAAAAAC (SEQ |
| | | | (SEQ ID NO: 139) | ID NO: 140) |
| KLK3 ex 1-2 | kallikrein | NM_001030048.1 | TGAGGAAGACAACCGGGA | AATCCGAGACAGGATGAGG |
| | related | (Accessed 5th | CCCACATGGTGACACAGC | GGTGCAGCACCAATCCACG |
| | peptidase 3 | Sep. 2019) | TCTCCGGGTG (SEQ ID | TCACGGACAGGG (SEQ |
| | | | NO: 141) | ID NO: 142) |
| KLK3 ex 2-3 | kallikrein | NM_001648.2 | ATCACGCTTTTGTTCCTG | CCTGTGTCTTCAGGATGAA |
| | related | (Accessed 5th | ATGCAGTGGGCAGCTGTG | ACAGGCTGTGCCGACCCAG |
| | peptidase 3 | Sep. 2019) | (SEQ ID NO: 143) | CAAG (SEQ ID NO: |
| | | | | 144) |
| KLK4 | kallikrein | NM_004917.3 | CCCAGCCAGAAACGAGGC | CAGCACGGTAGGCATTCTG |
| | related | (Accessed 5th | AAGAGTTCCCCGCGGTAG | CCGTTCGCCAGCAGAC |
| | peptidase 4 | Sep. 2019) | (SEQ ID NO: 145) | (SEQ ID NO: 146) |
| LBH | limb bud | NM_030915.3 | GAGAGTATGGATGAACCA | ACAGGAATTGAAAAGGCAA |
| | and heart | (Accessed 5th | CTCTCTGCAGCCAAAACA | GACCCCCGTCCACAAGGGG |
| | development | Sep. 2019) | GAACGAAGCGGGGA | AGGCGAGGGAAT (SEQ |
| | | | (SEQ ID NO: 147) | ID NO: 148) |
| POTEH-AS1 | POTEH | NR_110505.1 | ATTTATTTTACCCCCTAG | TCTTACCATTATTATTAAT |
| | antisense | (Accessed 5th | CTGATTTTCTATTACAGC | CTTACTTGCTTTCAGCATG |
| | RNA 1 | Sep. 2019) | ATATCAGTCTAGGG | CAGAGAGCTCTT (SEQ |
| | (POTEH-AS1), | | (SEQ ID NO: 149) | ID NO: 150) |
| | long | | | |
| | non-coding | | | |
| | RNA. | | | |
| | prostate- | | | |
| | specific P712P | | | |
| | mRNA | | | |
| MAK | male germ | NM_005906.3 | TATCTCCAGACTTGAAGA | CTTCTTGGAATGGGAGGCT |
| | cell | (Accessed 5th | TAGTCTGACCCCAACGCC | CCGAAATCATAGTCCTCCA |
| | associated | Sep. 2019) | TCCTACCACTTTTA | ACTCTTCCCAGC (SEQ |
| | kinase | | (SEQ ID NO: 151) | ID NO: 152) |
| MAPK8IP2 | mitogen- | NM_012324.2 | CTCTCGCTCCTCGCCGTT | CCGCGGGATGAACCTGAAC |
| | activated | (Accessed 5th | GACCAGACAGGAGAAAAG | ACAGCCCGGTGAGTCTG |
| | protein | Sep. 2019) | GCCAAAGGACTCG (SEQ | (SEQ ID NO: 154) |
| | kinase 8 | | ID NO: 153) | |
| | interacting | | | |
| | protein 2 | | | |
| MARCH5 | membrane | NM_017824.4 | TGTGCTGAAACTAGACTG | AAACAAAGAGCTCAAGGCC |
| | associated | (Accessed 5th | TCAACTCTGTAAGAGCTT | TCACCTTGGTTTATTCACT |
| | ring-CH-type | Sep. 2019) | GGACCAAGTCTGTC | GCTGGTTTTCTA (SEQ |
| | finger 5 | | (SEQ ID NO: 155) | ID NO: 156) |
| MCM7 | minichromosome | NM_182776.1 | TGTGTTCTCTCCTTCTAC | CAAGAAAATACCAGTGACG |
| | maintenance | (Accessed 5th | CAGCACCGTGATACTACG | CTGACGTGGTCTCCAGGCT |
| | complex | Sep. 2019) | AGGGATATT (SEQ ID | GGGCAATCCT (SEQ ID |
| | component | | NO: 157) | NO: 158) |
| | 7 | | | |
| MCTP1 | multiple C2 | NM_024717.4 | AACTCCAATTGTGTCAGA | GATAATGAGGATCTTTCAG |
| | and | (Accessed 5th | TCCAGAAAGGCTGAGCCC | AGTAAGGGTCACATCTGTG |
| | transmembrane | Sep. 2019) | ATAAAGTCATCCTG | GGCCTGTTT (SEQ ID |
| | domain | | (SEQ ID NO: 159) | NO: 160) |
| | containing 1 | | | |
| MDK | midkine | NM_001012334.1 | CGAGCAGACAGAAGGCAC | GGGGCTGGGGAGTGAGAGG |
| | (neurite | (Accessed 5th | TGGTGGGTCACATCTCGG | GACAAGGCAGGGCATGATT |
| | growth-pro | Sep. 2019) | GC (SEQ ID NO: | GATTAAAGCTAA (SEQ |
| | moting | | 161) | ID NO: 162) |
| | factor 2) | | | |
| MED4 | mediator | NM_001270629.1 | TCTTGCTTTTTCTATTGA | CTGATCCTATGTGCATACT |
| | complex | (Accessed 5th | CTTGAGTTTCTCCTTCGC | TAATTATTTCTTCAGAGGA |
| | subunit 4 | Sep. 2019) | TTGGTAAACAGCTG | GATAGCACCTTT (SEQ |
| | | | (SEQ ID NO: 163) | ID NO: 164) |
| MEMO1 | mediator of | NM_001137602.1 | GAATGTGCAGGTGGCATC | TATCGTGGTAAAGGCTAGG |
| | cell motility | (Accessed 5th | CCTGAGGATTCAGAGCT | CTGGGACCCCGGACAGAGT |
| | 1 | Sep. 2019) | (SEQ ID NO: 165) | ATGA (SEQ ID NO: |
| | | | | 166) |
| MET | MET | NM_001127500.1 | AAATTTATTATTCCTCCG | GTCAAGGTGCAGCTCTCAT |
| | proto- | (Accessed 5th | AAATCCAAAGTCCCAGCC | TTCCAAGGAGAACTCTAGT |
| | oncogene, | Sep. 2019) | ACATATGGTCAGCC | TTTCTTTAAATC (SEQ |
| | receptor | | (SEQ ID NO: 167) | ID NO: 168) |
| | tyrosine | | | |
| | kinase | | | |
| MEX3A | mex-3 RNA | NM_001093725.1 | GATCTATGCAACTTCTGA | CCTTTCAGCCACAGAAACG |
| | binding | (Accessed 5th | TAGGACTCCAACTCCCTT | ATTGACATGCTTCTCTCCC |
| | family | Sep. 2019) | ACACTGCTGGAAAC | CAACCCCTAGAA (SEQ |
| | member A | | (SEQ ID NO: 169) | ID NO: 170) |
| MFSD2A | major | NM_032793.4 | AAGAGGCAATAGAAAAGC | ACATGGTGAGAGCCGAGTA |
| | facilitator | (Accessed 5th | AGGTACCAATAGGTCTGG | GGGAACATGGAAACACGTG |
| | superfamily | Sep. 2019) | CCGTGTGGGAAGTC | ACCATTGTTTCA (SEQ |
| | domain | | (SEQ ID NO: 171) | ID NO: 172) |
| | containing | | | |
| | 2A | | | |
| MGAT5B | mannosyl | NM_144677.2 | GGTTGGAACAAGCAGGAG | CAGGTCATGCCAGGATGGG |
| | (alpha-1,6-)- | (Accessed 5th | AGAGAAACAATTCAACCA | TTTTGGGAGAAGCCCAGAG |
| | glycoprotein | Sep. 2019) | GGGTCTGGGTGGTC | TGAAAAG (SEQ ID NO: |
| | beta-1,6-N- | | (SEQ ID NO: 173) | 174) |
| | acetyl-glucosa- | | | |
| | minyltransferase, | | | |
| | isozyme B | | | |
| MIR146A | microRNA | ENST00000517927.1 | CGGTTGAGATTTCACCAA | TTCTGGATTTTCTCCATCA |
| | 146a | (Accessed 5th | GGTTCTGGTTCTGGAATG | GTCTAGGACTGAAGACACC |
| | | Sep. 2019) | AGTCACTGGCTAAG | GATCTCTGGTGT (SEQ |
| | | | (SEQ ID NO: 175) | ID NO: 176) |
| MIR4435-2HG | MIR4435-2 | ENST00000409569b.1 | AAAGCAGCGACCATCCAG | CAGGCACGGGCTCAGGCAC |
| | host gene | (Accessed | TCATTTATTTCCCTCCAT | CGCTTGTCTGGAATGTCAA |
| | | 5th Sep. | TCCCAATGATGTAC | TTTGAAACTTAA (SEQ |
| | | 2019) | (SEQ ID NO: 177) | ID NO: 178) |
| MKI67 | marker of | NM_002417.2 | CTGATGGCATTAGATTCC | GTCTTTCTCTTCACCTACT |
| | proliferation | (Accessed 5th | TGCACGCTAAGAGTTCTC | GATGGTTTAGGCGTGTGCA |
| | Ki-67 | Sep. 2019) | CCTCTACATCTG (SEQ | TGGCTTTGCCTG (SEQ |
| | | | ID NO: 179) | ID NO: 180) |
| MME | membrane | NM_000902.2 | TAGGGCTGGAACAAGGAC | CCAAAGGAATATTGCAAAT |
| | metalloendo | (Accessed 5th | TCTTTTCTCTGGACAGCT | ACCCAAGGTCACCCTGTCA |
| | peptidase | Sep. 2019) | TGCACCTACAATCC | GGAGTGGCAGAA (SEQ |
| | | | (SEQ ID NO: 181) | ID NO: 182) |
| MMP11 | matrix | NM_005940.3 | TCAGTGGGTAGCGAAAGG | ATATAGGTGTTGAACGCCC |
| | metallopeptidase | (Accessed 5th | TGTAGAAGGCGGACATCA | CTGCAGTCATCTGGGCTGA |
| | 11 | Sep. 2019) | GGGCCTTGG (SEQ ID | GAC (SEQ ID NO: |
| | | | NO: 183) | 184) |
| MMP25 | matrix | NM_022468.4 | CATTTAGATCCTAAAACT | CCCAGTGATTCTGATGTGG |
| | metallopeptidase | (Accessed 5th | GTGGGGAGTGGGGACAGG | GATAGTCTAGAAGAATAGT |
| | 25 | Sep. 2019) | GTGAACGAGGTGCC | TCCAGAGGCAAT (SEQ |
| | | | (SEQ ID NO: 185) | ID NO: 186) |
| MMP26 | matrix | NM_021801.3 | CAGGATTTCCAGAATTTG | TCCAGTGTCTGAAGCTGAC |
| | metallopeptidase | (Accessed 5th | GTAAAAAGGCATGGCCTA | CAGTGTTCATTCTTGTCAA |
| | 26 | Sep. 2019) | AGATACCACCTGGC | AATGGACAACTC (SEQ |
| | | | (SEQ ID NO: 187) | ID NO: 188) |
| MNX1 | motor | NM_005515.3 | TTTCTTGAAGAGCAGGTG | TTAAAAGAACCAGAGTTCA |
| | neuron and | (Accessed 5th | AGGCGCCCTTGCTTAAAA | AGTTTCAGCCCCCTGGGTC |
| | pancreas | Sep. 2019) | GGGAAGCGCCCAGG | TCCCTCTCGCTG (SEQ |
| | homeobox 1 | | (SEQ ID NO: 189) | ID NO: 190) |
| MSMB | microsemino | NM_002443.2 | TTTTTGGGTCCTTCTTCT | GTGCCTACTAGAAGCACAT |
| | protein beta | (Accessed 5th | CCACCACGATATACTTGC | TAGATTATCCATTCACTGA |
| | | Sep. 2019) | AGTCCTCCTTCTTG | CAGAACAGGTCT (SEQ |
| | | | (SEQ ID NO: 191) | ID NO: 192) |
| MXI1 | MAX | NM_001008541.1 | GAAGTGAATGAAAGTTTG | TGGCCCAGTGAATATTTTG |
| | interactor 1, | (Accessed 5th | ACACTGGCACTGGAGTAA | CCCTGCACTGTTATGTCAT |
| | dimerization | Sep. 2019) | CCCTCGTCACTCCC | GCTGGGTTCTAT (SEQ |
| | protein | | (SEQ ID NO: 193) | ID NO: 194) |
| MYOF | myoferlin | NM_013451.3 | ATGATCGTGTGACGCAAG | TGAGGTCCGGAATCATGTC |
| | | (Accessed 5th | TCAAGTTCTAGGAAACCC | CAATCTGCATTTCTCTGGT |
| | | Sep. 2019) | AAGTAGTCATCCAG | GATTTTGCAGGA (SEQ |
| | | | (SEQ ID NO: 195) | ID NO: 196) |
| NAALADL2 | N-acetylated | NM_207015.2 | ATTCTCAGCACCGTCTAG | TGAATGGAATCAAGATTGA |
| | alpha-linked | (Accessed 5th | CTGGAATTGGTCAAAACC | GGTCTATAGTCTCTGAATG |
| | acidic | Sep. 2019) | AGACTCCTCTAGTT | CCCTAGGTTCTG (SEQ |
| | dipeptidase | | (SEQ ID NO: 197) | ID NO: 198) |
| | like 2 | | | |
| NEAT1 | nuclear | NR_028272.1 | TTTCTCACACACAGATTT | TTCTCCTAGTAATCTGCAA |
| | paraspeckle | (Accessed 5th | AGGAATGACCAACTTGTA | TGCAATCACAATGCCCAAA |
| | assembly | Sep. 2019) | CCCTCCCAGCGTTT | CTAGACCTGCCA (SEQ |
| | transcript 1 | | (SEQ ID NO: 199) | ID NO: 200) |
| | (non-protein | | | |
| | coding) | | | |
| NKAIN1 | Na+/K+ | NM_024522.2 | CACTGTGTTCAAGGCCCA | GAACTCAGAGAGCAGACAC |
| | transporting | (Accessed 5th | CTTCCACCAAAAATCTAG | TGGGTTTTACAGTCAGAAA |
| | ATPase | Sep. 2019) | CTGTGTGGCCTCAA | CTGCAGAAAGTA (SEQ |
| | interacting 1 | | (SEQ ID NO: 201) | ID NO: 202) |
| NLRP3 | NLR family | NM_001079821.2 | CTGGCATATCACAGTGGG | CTCGAAAGGTACTCCAGTA |
| | pyrin | (Accessed 5th | ATTCGAAACACGTGCATT | AACCCATCCACTCCTCTTC |
| | domain | Sep. 2019) | ATCTGAACCCCACT | AATGCTGTCTTC (SEQ |
| | containing 3 | | (SEQ ID NO: 203) | ID NO: 204) |
| OGT | O-linked | NM_181672.1 | CTTTGAGAGCATTGGCTA | ACGGAGAGCTGTATTATAA |
| | N-acetylgluc | (Accessed 5th | GGTTGCAGTAAGCATCAG | CAATCTTCTGCTTCAGCAA |
| | osamine | Sep. 2019) | GGAAATGTGGTTGT | CACTGCCCTTCT (SEQ |
| | (GlcNAc) | | (SEQ ID NO: 205) | ID NO: 206) |
| | transferase | | | |
| OR51E2 | olfactory | NM_030774.2 | GAGCGTGCAGGCTGCGTT | GGATAAGGCCAGGTCAATG |
| | receptor | (Accessed 5th | CCGTCCTTACGATGAAGA | GCTGCAAGCATGCAGAGAA |
| | family 51 | Sep. 2019) | CCACGATGCAGTTT | AGAGGTACATCG (SEQ |
| | subfamily E | | (SEQ ID NO: 207) | ID NO: 208) |
| | member 2 | | | |
| PALM3 | paralemmin | NM_001145028.1 | AGCTGGGACTGGAGTGTG | GCTGGGCACCTGTGGAAGC |
| | 3 | (Accessed 5th | AACAAACTGTCTTCCAGG | ACTTTGCAACAGTTGC |
| | | Sep. 2019) | TTCCG (SEQ ID NO: | (SEQ ID NO: 210) |
| | | | 209) | |
| PCA3 | prostate | NR_015342.1 | TAAGGAACACATCAATTC | TCCCGTTCAAATAAATATC |
| | cancer | (Accessed 5th | ATTTTCTAATGTCCTTCC | CACAACAGGATCTGTTTTC |
| | associated 3 | Sep. 2019) | CTCACAAGCGGGAC | CTGCCCATCCTT (SEQ |
| | (non-protein | | (SEQ ID NO: 211) | ID NO: 212) |
| | coding) | | | |
| PCSK6 | proprotein | NM_138320.1 | ACATCGCCGTCCAGCATG | CGATGTAGTTGGGTCTGAT |
| | convertase | (Accessed 5th | CGGATGCCTCCTATTTTG | GCCCAGCGACTTTGCCTCG |
| | subtilisin/ | Sep. 2019) | GCATTGTACGCTAT | ACCACATCTGTG (SEQ |
| | kexin type 6 | | (SEQ ID NO: 213) | ID NO: 214) |
| PDLIM5 | PDZ and LIM | NR_046186.1 | CTCAAAGTCCAATGACAG | GGCCAACCAGTGACACACT |
| | domain 5 | (Accessed 5th | AAAATGAAATATGCTCGG | GTAGTTGCTCATGGTTCTA |
| | | Sep. 2019) | GTCCGGCGCGGCGC | ATGG (SEQ ID NO: |
| | | | (SEQ ID NO: 215) | 216) |
| PLPP1 | phospholipid | NM_176895.1 | GTGATTGCTCGGATAGTG | TTAGAAAACAGGCCAGCTT |
| | phosphatase | (Accessed 5th | ATTCCCAGTTGTTGGTGT | CACCTGGGCACCCTGCTGC |
| | 1 | Sep. 2019) | TTCATGCAGAGTTG | CTTTCAAGGCTG (SEQ |
| | | | (SEQ ID NO: 217) | ID NO: 218) |
| PPFIA2 | PTPRF | NM_003625.2 | CACTTTCATCCAGTCGCC | AGGAGGAAACTGCCTTCTC |
| | interacting | (Accessed 5th | TTTCAGTTCCCAGGGCCA | CAGGTTGATCCACGTCTGA |
| | protein | Sep. 2019) | AGAGGTTATTGTAT | AGTTCTTGTCAT (SEQ |
| | alpha 2 | | (SEQ ID NO: 219) | ID NO: 220) |
| PPP1R12B | protein | NM_001167857.1 | TGCTCTGTGATACTACTC | CTAGCAGAAGAGGCAGAGA |
| | phosphatase | (Accessed 5th | TTGCTTTCAGAGTTGGAA | AGGTATTTTGAGCTGGTGC |
| | 1 regulatory | Sep. 2019) | TGATTGACAAAGGC | TGGTATC (SEQ ID NO: |
| | subunit 12B | | (SEQ ID NO: 221) | 222) |
| PSTPIP1 | proline-serin | XM_006720737.1 | TCAAAGGAGGCCCTCAGG | AGCTGCCCACATTCTCCAT |
| | e-threonine | (Accessed 5th | GAGTTGATCTCCGTCTG | TTGCTGCTTCAAGGAG |
| | phosphatase | Sep. 2019) | (SEQ ID NO: 223) | (SEQ ID NO: 224) |
| | interacting | | | |
| | protein 1 | | | |
| PTN | pleiotrophin | NM_002825.5 | TTTCTTCCCTGCTTCAGC | CCATTCTCCACAGTCAGAC |
| | | (Accessed 5th | AGTATCCACAGCTGCCAG | TTCTTCACTTTTTTTTCTG |
| | | Sep. 2019) | TATGAAAATGAATG | GTTTCTC (SEQ ID NO: |
| | | | (SEQ ID NO: 225) | 226) |
| PTPRC | protein | NM_080923.2 | CAAGAGTTTAAGCCACAA | CTTTGCCCTGTCACAAATA |
| | tyrosine | (Accessed 5th | ATACATGGTCATATCTGG | CTTCTGTGTCCAGAAAGGC |
| | phosphatase, | Sep. 2019) | AAGTCAGCCGTGTC | AAAGCCAAATGC (SEQ |
| | receptor | | (SEQ ID NO: 227) | ID NO: 228) |
| | type C | | | |
| PVT1 | Pvt1 | NR_003367.2 | AAAATACTTGAACGAAGC | AGCGTTATTCCCCAGACCA |
| | oncogene | (Accessed 5th | TCCATGCAGCTGACAGGC | CTGAAGATCACTGTAAATC |
| | (non-protein | Sep. 2019) | ACAGCCATCTTGAG | CATCAGGCTCAG (SEQ |
| | coding) | | (SEQ ID NO: 229) | ID NO: 230) |
| RAB17 | RAB17, | NR_033308.1 | ACAGCACTTTCCTGGGAG | GGAACAGGCACAGGCATCG |
| | member RAS | (Accessed 5th | CCATGTGACGCCAGATCT | GGGAATCAGATGGTATCAG |
| | oncogene | Sep. 2019) | TCCTCTGGCAGTTC | TGGGGATAGGGC (SEQ |
| | family | | (SEQ ID NO: 231) | ID NO: 232) |
| RIOK3 | RIO kinase 3 | NM_003831.3 | CTGGAAAAACTGCGAGAC | ACAGCATTGAAGAGTTCTC |
| | | (Accessed 5th | ATTCCTGCAGTCCCGGAA | GTTCACTAAGGGCTTCCTT |
| | | Sep. 2019) | CAAGAACTCCAGGC | GACTCCTCCTTT (SEQ |
| | | | (SEQ ID NO: 233) | ID NO: 234) |
| RNF157 | ring finger | NM_052916.2 | ACTAGAGGGTAAACTTCT | CATGGCAATGGCCAAAATA |
| | protein 157 | (Accessed 5th | CGGTCTAAATCAAAGCCA | CTCGTCTCCTTCATCCACC |
| | | Sep. 2019) | AGCTCCTCTTCGGC | ACGGCATGTACC (SEQ |
| | | | (SEQ ID NO: 235) | ID NO: 236) |
| MRPL46 | mitochondrial | ENST00000561140.1 | TTGCCAGTCGCTGGTTTT | CAGCAATATATCCTGTTCA |
| | ribosomal | (Accessed 5th | CATCCAGAGCACGAAGCT | TCTTCTTCATCATGAAGGT |
| | protein L46 | Sep. 2019) | CGTGGTCTGAATAC | CAGCTTTCTTCT (SEQ |
| | | | (SEQ ID NO: 237) | ID NO: 238) |
| RPL18A | ribosomal | NM_000980.3 | GAGATACAAAGTACCAGA | CTGCCCACAGTAGACAATC |
| | protein L18a | (Accessed 5th | AGCGGGACTTGGCGACGA | TCCCCTGAAGACTTCTTCA |
| | | Sep. 2019) | CATGATTAGGCGCA | TCTTCTTTAACT (SEQ |
| | | | (SEQ ID NO: 239) | ID NO: 240) |
| RPL23AP53 | ribosomal | NR_003572.2 | AAATCCGAAAGGATCTCA | CATTTATGGCTGTCAACCC |
| | protein L23a | (Accessed 5th | TCCCATTAGGACCCTTGT | GCCAGTTCTCAGGAGTTTG |
| | pseudogene | Sep. 2019) | CTCCTTTTCTGTTG | TATAAAAGCCT (SEQ ID |
| | 53 | | (SEQ ID NO: 241) | NO: 242) |
| RPLP2 | ribosomal | NM_001004.3 | CTGATAACCTTGTTGAGC | TGCCAATACCCTGGGCAAT |
| | protein | (Accessed 5th | CGGTCGTCGTCCGCCTCG | GACGTCTTCAATGTTTTTT |
| | lateral stalk | Sep. 2019) | ATAC (SEQ ID NO: | CCATTCAGCTCA (SEQ |
| | subunit P2 | | 243) | ID NO: 244) |
| RPS10 | ribosomal | NM_001014.3 | GAAATGTCTCCAGGCAAA | TGAAGGTAATCACGGAGAT |
| | protein S10 | (Accessed 5th | CTGTTCCTTCACGTAGCC | ACTGGATACCCTCATTGGT |
| | | Sep. 2019) | TCGGGACTTGAGAG | AAGGTACCAGTA (SEQ |
| | | | (SEQ ID NO: 245) | ID NO: 246) |
| RPS11 | ribosomal | NM_001015.3 | CAGCAGGACCCTCTTCTT | AGACCGATGTTCTTGTAGT |
| | protein S11 | (Accessed 5th | GTTTTGAAAGATGGTCGG | ACCGCGGGAGCTTCTCCTT |
| | | Sep. 2019) | CTGCTTTTGGTAGG | GCCAGTTTCTCC (SEQ |
| | | | (SEQ ID NO: 247) | ID NO: 248) |
| SACM1L | SAC1 | NM_014016.3 | AGAAAGTTCTCTTAGAAG | ATAAAGCCATGTAACACTG |
| | suppressor | (Accessed 5th | ATGACCATTCCATACAAA | GAAGGGCAAACCGATGAAC |
| | of actin | Sep. 2019) | CCGCTGATCTGCCC | CTCTGGCTGTGC (SEQ |
| | mutations | | (SEQ ID NO: 249) | ID NO: 250) |
| | 1-like (yeast) | | | |
| SCHLAP1 | SWI/SNF | NR_104320.1 | CCAGGTACATGGTGAAAG | ACCTTGTGTCCCCAGCATC |
| | complex | (Accessed 5th | TGCCTTATACAGGTTGAA | TAGATTGCTGAAAAAGATG |
| | antagonist | Sep. 2019) | TAAAAATCACTGCC | TAGATGTTGCTT (SEQ |
| | associated | | (SEQ ID NO: 251) | ID NO: 252) |
| | with | | | |
| | prostate | | | |
| | cancer 1 | | | |
| | (non-protein | | | |
| | coding) | | | |
| SEC61A1 | Sec61 | NM_013336.3 | CTCTAAGCCCAACCAGAA | GAGCTGATGACCCAAGTGG |
| | translocon | (Accessed 5th | GAGTCAGCTAGAAGAGCC | ACTAAACACGGAGCTAGCA |
| | alpha 1 | Sep. 2019) | AATAGGTGCACAGA | GAAACAGGCAGA (SEQ |
| | subunit | | (SEQ ID NO: 253) | ID NO: 254) |
| SERPINB5 | serpin family | NM_002639.4 | CGGGCCTGGAGTCACAGT | GAACAGATCAACGGCAAAA |
| | B member 5 | (Accessed 5th | TATCCTGGAAAATGCGTG | GCCGAATTTGCTAGTTGCA |
| | | Sep. 2019) | GAAAAGGAACAGGC | GGGCATCCATTG (SEQ |
| | | | (SEQ ID NO: 255) | ID NO: 256) |
| SFRP4 | secreted | NM_003014.2 | CAGCCTCTCTTCCCACTG | CCCGGCTGTTTTCTTCTTG |
| | frizzled | (Accessed 5th | TATGGATCTTTTACTAAG | TCCTGAACTGTTCTCCGCT |
| | related | Sep. 2019) | CTGATCTCTCCATT | GTTCCTG (SEQ ID NO: |
| | protein 4 | | (SEQ ID NO: 257) | 258) |
| SIM2 | single-minded | NM_005069.3 | TTAATGTAGGTCGTGCGC | ATCCGCAAGTCGGCGGCGG |
| | family | (Accessed 5th | ATTTGCCGGGCTCGGTGG | GGTCCAATTCAAACAGCTG |
| | bHLH | Sep. 2019) | CGCCGCAGCC (SEQ ID | TCTCTGCATAAA (SEQ |
| | transcription | | NO: 259) | ID NO: 260) |
| | factor 2 | | | |
| SIM2 | single-minded | NM_009586.3 | CTGCCACCCACCGCCATG | GAAGCAGAAAGAGGGCAAG |
| | family | (Accessed 5th | GCTGCTTCGGCTCCCGG | TTTGCCCAAAGCGTGAGGG |
| | bHLH | Sep. 2019) | (SEQ ID NO: 261) | TTCTGTCTCCAT (SEQ |
| | transcription | | | ID NO: 262) |
| | factor 2 | | | |
| SIRT1 | sirtuin 1 | NM_012238.4 | GGTGTGGGTGGCAACTCT | CTGGTGGTGAAGTTCTTTC |
| | | (Accessed 5th | GACAAATAAGCCAATTCT | TGGTGAACTTGAGTCTTCT |
| | | Sep. 2019) | TTTTGTGTTCGTGG | GAAACATGAAGA (SEQ |
| | | | (SEQ ID NO: 263) | ID NO: 264) |
| SLC12A1 | solute | NM_000338.2 | CCATATACAACAAATCCG | TCTAACTAGTAAGACAGGT |
| | carrier | (Accessed 5th | ATATGGATCCCTTTCTTG | GGGAGGTTCTTTGTGAGGA |
| | family 12 | Sep. 2019) | CCACGGGAAGGCTC | TTTCCAACCAAG (SEQ |
| | member 1 | | (SEQ ID NO: 265) | ID NO: 266) |
| SLC43A1 | solute | NM_003627.5 | TTGACTTCCTCAGGGGCA | CTTGTGGTCCAGGGCCAGC |
| | carrier | (Accessed 5th | GGAAAGGCTTCGATGGGC | CCACTCAGCTTGATCTTCT |
| | family 43 | Sep. 2019) | CAGTTGAGGGTGCA | TCGTGTAA (SEQ ID |
| | member 1 | | (SEQ ID NO: 267) | NO: 268) |
| SLC4A1 | solute | NM_000342.3 | CATCATCAGCATCCAGAC | CACTTCGTCGTATTCATCC |
| | carrier | (Accessed 5th | ACTGAAGCTCCACGTTCC | CGACCTTCCTCCTCATCAA |
| | family 4 | Sep. 2019) | TGAAGATGAGCGG (SEQ | AGGTTGCCTTGG (SEQ |
| | member 1 | | ID NO: 269) | ID NO: 270) |
| SMAP1 | small | NM_021940.3 | GAGTACTTTGCTGTTGAA | TGGTGCTTGTGAGGTAAAT |
| | ArfGAP 1 | (Accessed 5th | TGGTTCCTGTGCCATACA | GGTATATTTGTGGGTCCCA |
| | | Sep. 2019) | GAGATAAGATGGAG | TAAATACACCAG (SEQ |
| | | | (SEQ ID NO: 271) | ID NO: 272) |
| SMIM1 | small | ENST00000444870.1 | TTCATGGCGATGCCCAGC | GGTAGCCCAGGATGAAGAT |
| | integral | (Accessed 5th | TTGCCCGTGCACAGCCTC | GATCCAGAAGAGGGCCACG |
| | membrane | Sep. 2019) | TGGGAGAT (SEQ ID | CCGCCCAGCACC (SEQ |
| | protein 1 | | NO: 273) | ID NO: 274) |
| | (Vel blood | | | |
| | group) | | | |
| SNCA | synuclein | NM_007308.2 | ACTGGGAGCAAAGATATT | GGAACTGAGCACTTGTACA |
| | alpha | (Accessed 5th | TCTTAGGCTTCAGGTTCG | GGATGGAACATCTGTCAGC |
| | | Sep. 2019) | TAGTCTTGATACCC | AGATCTCAAGAA (SEQ |
| | | | (SEQ ID NO: 275) | ID NO: 276) |
| SNORA20 | Small | NR_002960.1 | CGTATAACTGCTCGTATC | ATGGTTACTTCATCTCAAT |
| | nucleolar | (Accessed 5th | ACTGTGAGACTACAAGCA | TTACAGTGGCCCAATGTTA |
| | RNA | Sep. 2019) | GCAAATAAATGGGA | TTTTATCCCATG (SEQ |
| | SNORA20 | | (SEQ ID NO: 277) | ID NO: 278) |
| SPINK1 | serine | NM_003122.2 | AAGTTCTGCGTCCAGAGG | CAACAGGGCCAAGGCACTG |
| | peptidase | (Accessed 5th | TCAGTTGAAAACTGCACC | AGAAGAAAGATGCCTGTTA |
| | inhibitor, | Sep. 2019) | GCACTTACCACGTC | CCTTCATGGCTG (SEQ |
| | Kazal type 1 | | (SEQ ID NO: 279) | ID NO: 280) |
| SPON2 | spondin 2 | NM_012445.1 | CATTTATTCACTTCTCAA | AACGCAGAGAGATCCATAA |
| | | (Accessed 5th | GTGGCCCCCGCTTGGATG | CATGGAAACACTGACGCTT |
| | | Sep. 2019) | CGCCCTCG (SEQ ID | CCGAAACCGCCC (SEQ |
| | | | NO: 281) | ID NO: 282) |
| SRSF3 | serine and | NM_003017.4 | TAAAGTAACTGCCAACTG | CCATGTTCTAAAGTTTCTA |
| | arginine rich | (Accessed 5th | GGACTGTATGTCACCTAA | AGAGTCTTGAGGTTATGCT |
| | splicing | Sep. 2019) | GTCAGGATAACTCC | AGGGCTCCTGGT (SEQ |
| | factor 3 | | (SEQ ID NO: 283) | ID NO : 284 |
| SSPO | SCO-spondin | NM_198455.2 | CCACAAGGCAGGGAGAGA | ATGGTAGGCATCATGAAGG |
| | | (Accessed 5th | AGGGAGCCACATAAGTAG | GCACAGTGCTCGCTGC |
| | | Sep. 2019) | ATTCCTGGCG (SEQ ID | (SEQ ID NO: 286) |
| | | | NO: 285) | |
| SSTR1 | somatostatin | NM_001049.2 | TCCGACCCCGCAATCTTA | GGTCTTTGAAAACGCGCAG |
| | receptor 1 | (Accessed 5th | TAAAAACTCCTCATTCGG | TAGGAGGGTGATTCCTATT |
| | | Sep. 2019) | CTTGTTCTCAGCTC | ACGCGCCCACAC (SEQ |
| | | | (SEQ ID NO: 287) | ID NO: 288) |
| ST6GALNAC1 | ST6 | ENST0000059204 | TTTTTCCTCAAAATCCCA | TTCACAGAGTCAGGGCAAG |
| | N-acetylgalacto- | 2.1 (Accessed 5th | CCGAGGCTCAGATTTGAA | TCGTCTGAAGGCCTCCTAT |
| | saminide | Sep. 2019) | GTTGGCGGCCTTCA | TTCGAAGCTGTA (SEQ |
| | alpha-2,6- | | (SEQ ID NO: 289) | ID NO: 290) |
| | sialyltransferase | | | |
| | 1 | | | |
| STEAP2 | STEAP2 | NM_152999.2 | ATATATAAACCTGCCGGC | CTGGCGGGCAAGTTCAATA |
| | metalloreductase | (Accessed 5th | TGGCATCCTTAGGTCCTA | ACCTGTTGTCGCGCTTGAA |
| | | Sep. 2019) | ACTGAAGTGCCCAA | TATTGTTGCTGC (SEQ |
| | | | (SEQ ID NO: 291) | ID NO: 292) |
| STEAP4 | STEAP4 | NM_024636.2 | ATCAAAGATAAGTTGAAG | CCATGACTCTACTCAATGT |
| | metalloreductase | (Accessed 5th | GAGCGTGTGTTCTGTGTA | CGTCCAACTTTTTGTATCC |
| | | Sep. 2019) | CCTTTGCAACCAGT | TTGCTTGGGTTT (SEQ |
| | | | (SEQ ID NO: 293) | ID NO: 294) |
| STOM | stomatin | NM_004099.5 | GAGTCGGGGAGCCGCTGG | CCAAAATCCATCCGCAAGG |
| | | (Accessed 5th | GCTTCGGAGTCCCGTGT | TCCAAGGCCCTTACTGGGG |
| | | Sep. 2019) | (SEQ ID NO: 295) | CTGTCCTTGAAG (SEQ |
| | | | | ID NO: 296) |
| SULF2 | sulfatase 2 | NM_001161841.1 | ATGAGGTCTGTGAGGTAA | GTACATCTTCTTGGACGTG |
| | | (Accessed 5th | TCCTTGGAGTAGTCGGAG | CGGAAGAAGCTCACGCTGT |
| | | Sep. 2019) | C (SEQ ID NO: 297) | CATTGGTG (SEQ ID |
| | | | | NO: 298) |
| SULT1A1 | sulfotransferase | NM_177534.2 | CCCTCAATTCATATTTTA | TCAGCCTCCAAATTGCTGG |
| | family | (Accessed 5th | TTCTTGAGCCGCTTGGTC | GATTACAGACATGACCTAC |
| | 1A member | Sep. 2019) | AGGTTTGATTCGCA | CGTCCCGGG (SEQ ID |
| | 1 | | (SEQ ID NO: 299) | NO: 300) |
| SYNM | synemin | NM_015286.4 | AATGTGACATCGCTTTCT | TCGTGTTCTCCTGAGGCTG |
| | | (Accessed 5th | CCATAACCTTCCTCCTCC | CTTGGTCCTTCGATGCTGA |
| | | Sep. 2019) | TTAACCAACCCCCA | TTAACTGAG (SEQ ID |
| | | | (SEQ ID NO: 301) | NO: 302) |
| TBP | TATA-box | NM_001172085.1 | GCACGAAGTGCAATGGTC | TCCTCATGATTACCGCAGC |
| | binding | (Accessed 5th | TTTAGGTCAAGTTTACAA | AAACCGCTTGGGATTATAT |
| | protein | Sep. 2019) | CCAAGATTCACTGT | TCGGCGTTTCGG (SEQ |
| | | | (SEQ ID NO: 303) | ID NO: 304) |
| TDRD1 | Tudor | NM_198795.1 | TGTTTCTAGACTGTATAT | CCCAGCAACACACATCTGG |
| | domain | (Accessed 5th | CTGCTAACTGGCACCGTA | AATCTTGTTATGGCTTCTT |
| | containing 1 | Sep. 2019) | TTCCCTGAAAGGGA | CAGACCAATGTT (SEQ |
| | | | (SEQ ID NO: 305) | ID NO: 306) |
| TERF2IP | TERF2 | NM_018975.3 | GCCTGTGTAACTGTTGAT | ACGCTAAGAAGGCGGAAGT |
| | interacting | (Accessed 5th | AGATCCAAGTTAAACTTC | AGCCTCCAGCTCACCACTA |
| | protein | Sep. 2019) | TCCATTAACTGCCG | TTTTTTAGGAAG (SEQ |
| | | | (SEQ ID NO: 307) | ID NO: 308) |
| TERT | telomerase | NM_198253.1 | CGCAAGACCCCAAAGAGT | TCTGGAGGCTGTTCACCTG |
| | reverse | (Accessed 5th | TTGCGACGCATGTTCCTC | CAAATCCAGAAACAGGCTG |
| | transcriptase | Sep. 2019) | CCAGCCTTGAAGCC | TGACACTTCAGC (SEQ |
| | | | (SEQ ID NO: 309) | ID NO: 310) |
| TFDP1 | transcription | NM_007111.4 | TTCCTCTGCACCTTCTCG | TGAACTCCGCAACCAGCTC |
| | factor Dp-1 | (Accessed 5th | CAGACCTTCATGGAGAAA | GTCTGCCACTTCGTTGTAG |
| | | Sep. 2019) | TGCCGTAGGCCCTT | GAAGTGGTCCCT (SEQ |
| | | | (SEQ ID NO: 311) | ID NO: 312) |
| TIMP4 | TIMP | NM_003256.2 | TCTGCAGGGAAGGAGAAC | GGCACTTCTTATTAGCTGG |
| | metallopeptidase | (Accessed 5th | TGGCTTGATCTTCAGGAC | CAGCAAGAGGTCAGGTGGT |
| | inhibitor 4 | Sep. 2019) | TCTTGAAGGGATGT | AATGGCCAAAGC (SEQ |
| | | | (SEQ ID NO: 313) | ID NO: 314) |
| TMCC2 | transmembrane | NM_014858.3 | ACGTTGCTGCCGTCGGCC | CCCCGATGCCTTCGGCCTC |
| | and | (Accessed 5th | AGCAGCAGAGCAGTGTCG | CTCAGCCAGGAGGTAC |
| | coiled-coil | Sep. 2019) | GTG (SEQ ID NO: | (SEQ ID NO: 316) |
| | domain | | 315) | |
| | family 2 | | | |
| TMEM45B | transmembrane | NM_138788.3 | GCATACAGCAGGAGTGAG | GGTCCCGGAAGATCACCTC |
| | protein | (Accessed 5th | TGGATGTGCTGGTCCAGC | TAGGGAGATACTAACACAC |
| | 45B | Sep. 2019) | GGAGGCCGG (SEQ ID | CCTCCGAACAGA (SEQ |
| | | | NO: 317) | ID NO: 318) |
| TMEM47 | transmembrane | NM_031442.3 | AGCAAATAACCAACAGCC | CCCATTAGATGCTGAAGGG |
| | protein | (Accessed 5th | AATGTAGTCATTGGGTAG | CAGTTCATTTTTCAAGGGC |
| | 47 | Sep. 2019) | GATAAGCAGGCGGT | TCACTCA (SEQ ID NO: |
| | | | (SEQ ID NO: 319) | 320) |
| TMEM86A | transmembrane | NM_153347.1 | AATGAATCAGCCAATCTA | GCTCCTGGAGCAGAGTGAT |
| | protein | (Accessed 5th | ATCCCATTGCTCCCAGCT | GTATTATTCTGCCAGGGCT |
| | 86A | Sep. 2019) | GTTCAACTAAGCCC | TTACAACTAATG (SEQ |
| | | | (SEQ ID NO: 321) | ID NO: 322) |
| TRPM4 | transient | NM_001195227.1 | CTTCCAGTAGAGATCGCT | GCCAGCGCGGGCCGAGAGT |
| | receptor | (Accessed 5th | GTTGCCCTGTACTTTGCC | GGAATTCCCGGATGAGGCG |
| | potential | Sep. 2019) | GAATGTGTAACTGA | GTAACGCTGCGC (SEQ |
| | cation | | (SEQ ID NO: 323) | ID NO: 324) |
| | channel | | | |
| | subfamily M | | | |
| | member 4 | | | |
| TWIST1 | twist family | NM_000474.3 | CTCGGCGGCTGCTGCCGG | TGCTGCTGCGCCGCTTGCG |
| | bHLH | (Accessed 5th | TCTGGCTCTTCCTCGCTG | TCCCCCGCGCTTGCCG |
| | transcription | Sep. 2019) | (SEQ ID NO: 325) | (SEQ ID NO: 326) |
| | factor 1 | | | |
| UPK2 | uroplakin 2 | NM_006760.3 | ACGAGGTTTGTCACCTGG | TCCCCTTCTTCACTAGGTA |
| | | (Accessed 5th | TATGCACTGAGCCGAGTG | GGAAATGTAGAATTTGGTT |
| | | Sep. 2019) | ACTG (SEQ ID NO: | CCTGGC (SEQ ID NO: |
| | | | 327) | 328) |
| VAX2 | ventral | NM_012476.2 | TCACAGGGTGGGAGTCTT | ACAGGAGACTGGGAAGGTG |
| | anterior | (Accessed 5th | AAGTGTTAGCTTTCTTGC | CTGTGCTCGGGACTCAGTG |
| | homeobox 2 | Sep. 2019) | AG (SEQ ID NO: | (SEQ ID NO: 330) |
| | | | 329) | |
| VPS13A | vacuolar | NM_033305.2 | TAAAGGGCTTTGGTGCTG | ACGTGATATCTGGGAATGT |
| | protein | (Accessed 5th | AATCCATGGTGACCGACT | CCTGCAGATCTCATGACAA |
| | sorting 13 | Sep. 2019) | TTGGAGGTTTAACA | TACTGACATCTG (SEQ |
| | homolog A | | (SEQ ID NO: 331) | ID NO: 332) |
| ZNF577 | zinc finger | NM_032679.2 | TCTCTCTTCTGTCTATTC | GCCTTGCCCATTTCGTTCA |
| | protein 577 | (Accessed 5th | TGGGCCTTCCCAGAAGTG | ACTCTTAGGGGCTAGCAAC |
| | | Sep. 2019) | GTGGTCAG (SEQ ID | TCTAGTATGTTC (SEQ |
| | | | NO: 333) | ID NO: 334) |

### Methylation Analysis

Hypermethylation at the 5-regulatory regions of six genes (GSTP1, SFRP2, IGFBP3, IGFBP7, APC and PTSG2) in urinary cell-pellet DNA was assessed using quantitative methylation-specific PCR as described by O'Reilly et al (2019) [30].

DNA methylation of each gene is indicated by the NIM (normalised index of methylation), and for the collective panel, by the epiCaPture score (NIM sum G1-G6). For ease of interpretation, DNA methylation is plotted on a logarithmic axis, and samples with no methylation are not shown. DNA methylation was measured using the Infinium HumanMethylation450 BeadChip (HM450k). Genomic DNA is used in bisulfite conversion to convert the unmethylated cytosine into uracil. The product contains unconverted cytosine where they were previously methylated, but cytosine converted to uracil if they were previously unmethylated.

The bisulfite treated DNA is subjected to whole-genome amplification (WGA) via random hexamer priming and Phi29 DNA polymerase. The products are then enzymatically fragmented, purified from dNTPs, primers and enzymes, and applied to the chip.

On the chip, there are two bead types for each CpG (or “CG”, as per FIG. 1) site per locus. Each locus tested is differentiated by different bead types. Both bead types are attached to single-stranded 50-mer DNA oligonucleotides that differ in sequence only at the free end; this type of probe is known as an allele-specific oligonucleotide. One of the bead types will correspond to the methylated cytosine locus and the other will correspond to the unmethylated cytosine locus, which has been converted into uracil during bisulfite treatment and later amplified as thymine during whole-genome amplification. The bisulfite-converted amplified DNA products are denatured into single strands and hybridized to the chip via allele-specific annealing to either the methylation-specific probe or the non-methylation probe. Hybridization is followed by single-base extension with hapten-labeled dideoxynucleotides. The ddCTP and ddGTP are labeled with biotin while ddATP and ddUTP are labeled with 2,4-dinitrophenol (DNP).

After incorporation of these hapten-labeled ddNTPs, multi-layered immunohistochemical assays are performed by repeated rounds of staining with a combination of antibodies to differentiate the two types. After staining, the chip is scanned to show the intensities of the unmethylated and methylated bead types. The raw data are analyzed by the software, and the fluorescence intensity ratios between the two bead types are calculated. For a given individual at a given locus, a ratio value of 0 equals to non-methylation of the locus (i.e., homozygous unmethylated); a ratio of 1 equals to total methylation (i.e., homozygous methylated); and a value of 0.5 means that one copy is methylated and the other is not (i.e., heterozygosity), in the diploid human genome.

The scanned microarray images of methylation data are further analyzed by the system, which normalizes the raw data to reduce the effects of experimental variation, background and average normalization, and performs standard statistical tests on the results. The data can then be compiled into several types of figures for visualization and analysis. Scatter plots are used to correlate the methylation data; bar plots to visualize relative levels of methylation at each site tested; heat maps to cluster the data to compare the methylation profile at the sites tested.

TABLE 9
epiCapture qMSP primers and probes

| | Oligonucleotide Sequence (5′-3′) |
|---|---|
| Gene | Forward |
| GSTP1 (G1) | gtt gcg tgg cga ttt cg |
| SFRP2 (G2) | agt ttt tcg gag ttg cgc g |
| IGFBP3 (G3) | ttt ttt cga tat cgg ttc gtc g |
| IGFBP7 (G4) | aag cgg gcg tga gat cg |
| APC (G5) | tta tat gtc cgt tac gtg cgt tta tat |
| PTGS2 (G6) | cgg aag cgt tcg ggt aaa g |
| | Oligonucleotide Sequence (5′-3′) |
| Gene | Reverse |
| GSTP1 (G1) | cga act ccc gcc gac c |
| SFRP2 (G2) | gct ctc ttc gct aaa tac gac tcg |
| IGFBP3 (G3) | gat ctc ctt aac ccc gcc g |
| IGFBP7 (G4) | cgc gct cct act aac gtc g |
| APC (G5) | gaa cca aaa cgc tcc cca t |
| PTGS2 (G6) | gaa ttc cac cgc ccc aaa cg |
| | Oligonucleotide Sequence (5′-3′) |
| Gene | Probe |
| GSTPI(G1) | cga cga ccg cta cac |
| SFRP2 (G2) | tgt agc gtt tcg ttc gc |
| IGFBP3 (G3) | aga ttt tat ttc gag agc gga |
| IGFBP7 (G4) | tta tgg gtc ggt tac gtc g |
| APC (GS) | ccc gtc gaa aac ccg ccg att a |
| PTGS2 (G6) | ttt ccg cca aat atc ttt tct tct tcg ca |

### Protein Quantification

Urinary EN2 protein concentration was quantified by ELISA using a monoclonal anti-mouse EN2 antibody, as described by Morgan et al (2011) [72].

### EN2 Protein Detection—ELISA

Two monoclonal mouse anti-EN2 antibodies were raised using the synthetically produced C-terminal 100 amino acids (Biosynthesis Inc.) of EN2 as an antigen (Antibody Production Services Ltd.). One of these, APS1, was conjugated to alkaline phosphatase using the Lightning Link alkaline phosphatase conjugation kit (Innova Biosciences), whilst the other, APS2, was conjugated to biotin using the Lightning Link Biotin Conjugation kit (Innova Biosciences). APS2-biotin was captured onto a 96-well streptavidin-coated plate (Nunc 436014) at a concentration of 4 mg/mL. After washing, 100 mL of urine or a dilution of the EN2 fragment in buffer was incubated in each well for 1 hour at room temperature. The plate was then washed 8 times in buffer (PBS with 0.1% Tween-20) and the secondary detection antibody-APS1-alkaline phosphatase was added to each well at a concentration of 4 mg/mL (1 hour at room temperature). After a final wash step a colormetric agent—pNPP (Sigma) was added and the absorption of light at 405 nm was measured after 1 hour. The dilution series was used to generate a standard curve by which the concentration of EN2 in each sample was measured.

### Alternatively EN2 Protein can be Detected by Western Blotting

An amount of 1.5 mL urine was centrifuged at 10,000 g for 5 minutes to remove cells and cellular debris.

Twenty microliters of the supernatant were then mixed directly with gel running buffer (Invitrogen). Proteins were resolved by 10% SDS-polyacrylamide gel electrophoresis and transferred to a polyvinylidene fluoride membrane (Invitrogen). Anti-EN2 antibody (ab45867; Abcam) was used at concentration of 0.5 mg/mL, and a goat-anti human IgG peroxidize-labeled antibody was used together with the ECL chemiluminescent system for detection.

A sequence listing is provided with the present application for search purposes. In the event that there is any variation in the sequences in the description and the sequences in the sequence listing, the sequence in the description is to be used as the definitive version of the sequence.

All publications, patents and patent applications discussed and cited herein are incorporated herein by reference in their entireties. It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

While the invention has been described in terms of preferred embodiments, those skilled in the art will recognize that the invention can be practiced with modification within the scope of the appended claims.

### EMBODIMENTS

Further embodiments of the present invention are described below:
1. A method of providing a cancer diagnosis or prognosis based on one or more clinical variables and/or the expression status of a plurality of genes, comprising:
   (a) providing a plurality of patient profiles each comprising the one or more clinical variables and/or the expression status of the plurality of genes in at least one sample obtained from each patient, wherein each of the patient profiles is associated with one of (n) biopsy outcome groups, wherein each biopsy outcome group is assigned a risk score and is associated with a different cancer prognosis or cancer diagnosis;
   (b) applying a first supervised machine learning algorithm (for example random forest analysis) to the patient profiles to select a subset of one or more clinical variables and/or a subset of expression statuses of one or more genes from the plurality of genes in the patient profile that are associated with each biopsy outcome group;
   (c) inputting the values of the subset of one or more clinical variables and/or subset of expression statuses of one or more genes into a second supervised machine learning algorithm (for example random forest analysis) comprising one or more decision trees; (d) calculating a cut point for each of the one or more clinical variables and/or expression statuses of the one or more genes within the one or more decision trees to optimise the discrimination of each biopsy outcome group within the patient profiles, wherein the cut point can be used to generate a risk score for each decision tree;
   (e) calculating an average risk score for each patient using the risk scores from each decision tree in (d); and
   (f) providing a cancer diagnosis or prognosis for each patient or determining whether each patient has a poor prognosis based on whether the risk score for each patient is associated with a poor prognosis biopsy outcome group.
2. A method of providing a cancer diagnosis or prognosis based on one or more clinical variables and/or the expression status of a plurality of genes, comprising:
   (a) providing a reference dataset comprising a plurality of patient profiles each comprising the one or more clinical variables and expression status values of one or more genes in at least one sample obtained from each patient wherein the biopsy outcome group of each patient sample in the dataset is known and wherein each biopsy outcome group is assigned a risk score and is associated with a different cancer prognosis or cancer diagnosis;
   (b) using the one or more clinical variables and/or expression status values for one or more genes to apply a supervised machine learning algorithm (for example random forest analysis) to the reference dataset to obtain a predictor for biopsy outcome group;
   (c) determining the same one or more clinical variables and/or expression status values for the same one or more genes in a sample obtained from a test subject to provide a test subject profile;
   (d) applying the predictor to the test subject profile to generate a risk score for the test subject profile; and
   (e) providing a cancer diagnosis or prognosis for the test subject or determining whether the test subject has a poor prognosis based on whether the risk score for the test subject profile is associated with a poor prognosis biopsy outcome group.
3. A method of providing a cancer diagnosis or prognosis based on one or more clinical variables and/or the expression status of a plurality of genes, comprising:
   (a) providing a reference dataset comprising a plurality of patient profiles each comprising the one or more clinical variables and expression status values of one or more genes in at least one sample obtained from each patient wherein the biopsy outcome group of each patient sample in the dataset is known and wherein each biopsy outcome group is assigned a risk score and is associated with a different cancer prognosis or cancer diagnosis;
   (b) inputting the values of the one or more clinical variables and expression status values of one or more genes into a supervised machine learning algorithm (for example random forest analysis) comprising one or more decision trees;
   (c) calculating a cut point for each of the one or more clinical variables and/or expression status of the one or more genes within the one or more decision trees to optimise the discrimination of each biopsy outcome group within the patient profiles, wherein the cut point can be used to generate a risk score for each decision tree;
   (d) providing a test subject profile comprising values for the same one or more clinical variables and/or expression status of the same one or more genes in at least one sample obtained from the test subject;
   (e) inputting the test subject profile into the supervised machine learning algorithm comprising the calculated cut points to generate a test subject risk score for each decision tree;
   (f) calculating an average risk score for the test subject profile based on the risk scores for each decision tree calculated in step (e); and
   (g) providing a cancer diagnosis or prognosis for the test subject or determining whether the test subject has a poor prognosis based on whether the average risk score for the test subject profile is associated with a poor prognosis biopsy outcome group.
4. The method of any one of embodiments 2 or 3 wherein the one or more clinical variables and expression status values of one or more genes comprises the expression status of one or more of GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion and optionally PSA level (e.g. serum PSA level).
5. The method of any one of embodiments 2-4 wherein the expression status of one or more of GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion is determined by methylation status.
6. The method of any one of embodiments 2-5 wherein the expression status of one or more of GSTP1, APC, SFRP2, IGFBP3, IGFBP7, PTGS2 is determined by methylation status.
7. The method of any one of embodiments 2-6 wherein the expression status of all of GSTP1, APC, SFRP2, IGFBP3, IGFBP7, PTGS2 are determined by methylation status.
8. The method of any one of embodiments 2-7 wherein the expression status of all of GSTP1, APC, SFRP2, IGFBP3, IGFBP7, PTGS2 are determined by methylation status and the expression status of ERG exons 4-5, ERG exons 6-7, GJB1, HOXC6, HPN, PCA3, SNORA20, TIMP4 and TMPRSS2/ERG fusion are determined by RNA microarray.
9. The method of any one of embodiments 2 or 3 wherein the one or more clinical variables and expression status values of one or more genes comprises the expression status of one or more of EN2, ERG exons 4-5, ERG exons 6-7, GJB1, HOXC6, HPN, PCA3, PPFIA2, TMPRSS2/ERG fusion, SLC12A1 and TMEM45B fusion and optionally PSA level (e.g. serum PSA level).
10. The method of any one of embodiments 2, 3 or 9 wherein the expression status of one or more of EN2, ERG exons 4-5, ERG exons 6-7, GJB1, HOXC6, HPN, PCA3, PPFIA2, TMPRSS2/ERG fusion, SLC12A1 and TMEM45B fusion is determined by protein concentration.
11. The method of any one of embodiments 2, 3 or 9-10 wherein the expression status of EN2 is determined by protein concentration in the sample.
12. The method of any one of embodiments 2, 3 or 9-11 wherein the expression status of EN2 is determined by protein concentration in the sample and the expression status of ERG exons 4-5, ERG exons 6-7, GJB1, HOXC6, HPN, PCA3, PPFIA2, TMPRSS2/ERG fusion, SLC12A1 and TMEM45B fusion are determined by RNA microarray.
13. The method according to any preceding embodiment, wherein the biopsy outcome group is classified by Gleason score (Gs).
14. The method according to any preceding embodiment, wherein the number of possible biopsy outcome groups (n) is 1, 2, 3, 4, 5, 6, 7, 8, 9 or 10.
15. The method according to any preceding embodiment, wherein the n biopsy outcome groups comprise a group associated with no cancer diagnosis and one or more groups (e.g. 1, 2, 3 groups) associated with increasing risk of cancer diagnosis, severity of cancer or chance of cancer progression.
16. The method according to any preceding embodiment, wherein the higher a risk score is the higher the probability a given patient or test subject exhibits or will exhibit the clinical features or outcome of the corresponding biopsy outcome group.
17. The method according to any preceding embodiment, wherein at least one of the biopsy outcome groups is associated with a poor prognosis of cancer.
18. The method according to any preceding embodiment, wherein the number of biopsy outcome groups (n) is 4.
19. The method according to embodiment 18, wherein the 4 biopsy outcome groups are (i) no evidence of cancer, (ii) Gleason score (Gs)=6, (iii) Gleason score (Gs)=3+4 and (iv) Gleason score (Gs)≥4+3.
20. The method according to embodiment 1 or 13-19, wherein the step of selecting a subset of variables further comprises discarding any variables that are not associated with any of the n biopsy outcome groups.
21. The method according to any preceding embodiment, wherein the one or more clinical variables and/or expression status of the plurality of genes is selected from one of the lists in Table 1 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176 or 177 of the items in Table 1).
22. The method according to any preceding embodiment, wherein the one or more clinical variables and/or expression status of the plurality of genes is selected from the list in the ExoRNA column of Table 1 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166 or 167 of the items in the ExoRNA column of Table 1).
23. The method according to any preceding embodiment, wherein the one or more clinical variables and/or expression status of the plurality of genes is selected from the list in the ExoMeth column of Table 1 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176 or 177 of the items in the ExoMeth column of Table 1).
24. The method according to any preceding embodiment, wherein the one or more clinical variables and/or expression status of the plurality of genes is selected from the list in the ExoGrail column of Table 1 (i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171 or 172 of the items in the ExoGrail column of Table 1).
25. The method according to any one of embodiments 1-21 and 23, wherein the subset of one or more clinical variables and/or expression status of the plurality of genes is selected from the list of items in the ExoMeth column of Table 3 (i.e. 1, 2, 3, 4, 5, 6, 7, 8, 9, 10, 11, 12, 13, 14, 15 or 16 of the items in Table 3).
26. The method according to any one of embodiments 1-21 and 24, wherein the subset of one or more clinical variables and/or expression status of the plurality of genes is selected from the list of items in the ExoGrail column of Table 5 (i.e. 1, 2, 3, 4, 5, 6, 7, 8, 9, 10, 11 or 12 of the items in Table 5).
27. A method of diagnosing or testing for prostate cancer in a subject comprising determining the expression status of one or more genes selected from the group consisting of GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion in a biological sample from the subject, optionally wherein the serum PSA level of the subject is also used in the method of diagnosing or testing for prostate cancer.
28. A method of diagnosing or testing for prostate cancer in a subject comprising determining the expression status of one or more genes selected from the group consisting of EN2, ERG exons 4-5, ERG exons 6-7, GJB1, HOXC6, HPN, PCA3, PPFIA2, TMPRSS2/ERG fusion, SLC12A1 and TMEM45B fusion in a biological sample from the subject, optionally wherein the serum PSA level of the subject is also used in the method of diagnosing or testing for prostate cancer.
29. The method of any preceding embodiment wherein the expression status of one or more genes is determined by methylation status, optionally wherein the expression status of one or more of GSTP1, APC, SFRP2, IGFBP3, IGFBP7 and PTGS2 is determined by methylation status.
30. The method of embodiment 29 wherein the methylation status of the one or more genes is determined by methylation microarray.
31. The method of any preceding embodiment wherein the expression status of one or more genes is determined by protein quantification, optionally wherein the expression status of EN2 is determined by protein quantification.
32. The method of embodiment 31 wherein the expression status of one or more genes is determined by protein ELISA.
33. A method of diagnosing or testing for prostate cancer in a subject comprising determining the methylation status of one or more genes selected from the group consisting of GSTP1, APC, SFRP2, IGFBP3, IGFBP7, PTGS2, and the expression status of one or more genes selected from the group consisting of ERG exons 4-5, ERG exons 6-7, GJB1, HOXC6, HPN, PCA3, SNORA20, TIMP4 and TMPRSS2/ERG fusion in a biological sample from the subject, optionally wherein the serum PSA level of the subject is also used in the method of diagnosing or testing for prostate cancer.
34. A method of diagnosing or testing for prostate cancer in a subject comprising determining the expression status of EN2 by protein quantification and the expression of one or more genes selected from the group consisting of ERG exons 4-5, ERG exons 6-7, GJB1, HOXC6, HPN, PCA3, PPFIA2, TMPRSS2/ERG fusion, SLC12A1 and TMEM45B fusion in a biological sample from the subject, optionally wherein the serum PSA level of the subject is also used in the method of diagnosing or testing for prostate cancer.
35. The method according to any preceding embodiment wherein the expression status of one or more genes is determined by one or more methods including, protein quantification, methylation status, RNA extraction, RNA hybridisation or sequencing optionally wherein the expression status of EN2 is determined by protein quantification.
36. The method according to any preceding embodiment, wherein the method can be used to determine whether a patient should be biopsied.
37. The method according to embodiment 36, wherein the method is used in combination with MRI imaging data to determine whether a patient should be biopsied.
38. The method according to embodiment 37, wherein the MRI imaging data is generated using multiparametric-MRI (MP-MRI).
39. The method according to any one of embodiments 37-38, wherein the MRI imaging data is used to generate a Prostate Imaging Reporting and Data System (PI-RADS) grade.
40. The method according to any preceding embodiment, wherein the method can be used to predict disease progression in a patient.
41. The method according to any preceding embodiment, wherein the patient is currently undergoing or has been recommended for active surveillance.
42. The method according to embodiment 41, wherein the patient is currently undergoing active surveillance by PSA monitoring, biopsy and repeat biopsy and/or MRI, at least every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks or 24 weeks.
43. The method according to any preceding embodiment, wherein the method can be used to predict disease progression in patients with a Gleason score of ≤10, ≤9, ≤8, ≤7 or ≤6.
44. The method according to any preceding embodiment, wherein the method can be used to predict:
   (i) the volume of Gleason 4 or Gleason ≥4 prostate cancer; and/or
   (ii) low risk disease that will not require treatment for 1, 2, 3, 4, 5 or more years.
45. The method according to any preceding embodiment, wherein the biological sample is processed prior to determining the expression status of the one or more genes in the biological sample.
46. The method according to any preceding embodiment, wherein determining the expression status of the one or more genes comprises extracting RNA from the biological sample.
47. The method according to embodiment 46, wherein the RNA is extracted from extracellular vesicles.
48. The method according to any preceding embodiment wherein determining the expression status of the one or more genes comprises the step of quantifying the expression status of the RNA transcript or cDNA molecule and wherein the expression status of the RNA or cDNA is quantified using any one or more of the following techniques: microarray analysis, real-time quantitative PCR, DNA sequencing, RNA sequencing, Northern blot analysis, in situ hybridisation and/or detection and quantification of a binding molecule.
49. The method according to embodiment 48, wherein the determining the expression status of the RNA or cDNA comprises RNA or DNA sequencing.
50. The method according to embodiment 48, wherein the determining the expression status of the RNA or cDNA comprises using a microarray.
51. The method according to embodiment 50, further comprising the step of capturing the one or more RNAs or cDNAs on a solid support and detecting hybridisation.
52. The method according to embodiment 51, further comprising sequencing the one or more RNA or cDNA molecules.
53. The method according to any one of embodiments 50-52, wherein the microarray comprises a probe having a nucleotide sequence with at least 80%, 85%, 90%, 95%, 96%, 97%, 98% or 99% identity to a nucleotide sequence selected from any one of SEQ ID NOs 1 to 334.
54. The method according to any one of embodiments 50-52, wherein the microarray comprises a probe having a nucleotide sequence selected from any one of SEQ ID NOs 1 to 334.
55. The method according to any one of embodiments 50-52, wherein the microarray comprises 334 probes each having a nucleotide sequence with at least 80%, 85%, 90%, 95%, 96%, 97%, 98% or 99% identity to a unique nucleotide sequence selected from any one of SEQ ID NOs 1 to 334.
56. The method according to any one of embodiments 50-52, wherein the microarray comprises 334 probes, each having a unique nucleotide sequence selected from SEQ ID NOs 1 to 334.
57. The method according to any one of embodiments 50-52, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 277 and SEQ ID NO: 278, and SEQ ID NO: 313 and SEQ ID NO: 314.
58. The method according to embodiment 57, wherein the microarray comprises a pair of probes for every gene of interest having nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 277 and SEQ ID NO: 278, and SEQ ID NO: 313 and SEQ ID NO: 314.
59. The method according to any one of embodiments 50-52, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 219 and SEQ ID NO: 220, SEQ ID NO: 265 and SEQ ID NO: 266, and SEQ ID NO: 317 and SEQ ID NO: 318.
60. The method according to embodiment 59, wherein the microarray comprises a pair of probes for every gene of interest having nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 219 and SEQ ID NO: 220, SEQ ID NO: 265 and SEQ ID NO: 266, and SEQ ID NO: 317 and SEQ ID NO: 318.
61. The method according to any preceding embodiment, wherein determining the expression status of the one or more genes comprises extracting protein from the biological sample.
62. The method according to embodiment 61, wherein the protein is extracted directly from the biological sample.
63. The method according to any preceding embodiment, wherein determining the expression status of the one or more genes comprises determining the methylation status of one or more genes.
64. The method according to any preceding embodiment, further comprising the step of comparing or normalising the expression status of one or more genes with the expression status of a reference gene.
65. The method according to any preceding embodiment wherein the biological sample is a urine sample, a semen sample, a prostatic exudate sample, or any sample containing macromolecules or cells originating in the prostate, a whole blood sample, a serum sample, saliva, or a biopsy (such as a prostate tissue sample or a tumour sample).
66. The method according to any preceding embodiment wherein the biological sample is a urine sample.
67. The method according to any preceding embodiment wherein the sample is from a human.
68. A method of treating prostate cancer, comprising diagnosing a patient as having or as being suspected of having prostate cancer using a method as defined in any one of embodiments 1 to
67, and administering to the patient a therapy for treating prostate cancer.
69. A method of treating prostate cancer in a patient, wherein the patient has been determined as having prostate cancer or as being suspected of having prostate cancer according to a method as defined in any one of embodiments 1 to 67, comprising administering to the patient a therapy for treating prostate cancer.
70. The method according to embodiment 65 or 66, wherein the therapy for prostate cancer comprises surgery, brachytherapy, active surveillance, chemotherapy, hormone therapy, immunotherapy and/or radiotherapy.
71. The method according to embodiment 70, wherein the chemotherapy comprises administration of one or more agents selected from the following list: abiraterone acetate, apalutamide, bicalutamide, cabazitaxel, bicalutamide, degarelix, docetaxel, leuprolide acetate, enzalutamide, apalutamide, flutamide, goserelin acetate, mitoxantrone, nilutamide, sipuleucel-T, radium 223 dichloride and docetaxel.
72. The method according to embodiment 69 or 70, wherein the therapy for prostate cancer comprises resection of all or part of the prostate gland or resection of a prostate tumour.
73. An RNA, cDNA or protein molecule of one or more genes selected from the group consisting of: GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion for use in a method of diagnosing or testing for prostate cancer comprising determining the expression status of the one or more genes, optionally wherein the serum PSA level of the subject is also used in the method of diagnosing or testing for prostate cancer.
74. The RNA, cDNA or protein molecule for use of embodiment 73 wherein the expression status of one or more genes is determined by methylation status, optionally wherein the expression status of one or more of GSTP1, APC, SFRP2, IGFBP3, IGFBP7 and PTGS2 is determined by methylation status.
75. An RNA, cDNA or protein molecule of one or more genes selected from the group consisting of EN2, ERG exons 4-5, ERG exons 6-7, GJB1, HOXC6, HPN, PCA3, PPFIA2, TMPRSS2/ERG fusion for use in a method of diagnosing or testing for prostate cancer comprising determining the expression status of the one or more genes, optionally wherein the serum PSA level of the subject is also used in the method of diagnosing or testing for prostate cancer.
76. The RNA, cDNA or protein molecule for use of embodiment 75 wherein the expression status of one or more genes is determined by protein quantification, optionally wherein the expression status of EN2 is determined by protein quantification, further optionally wherein the expression status is determined by protein ELISA.
77. An RNA, cDNA or protein molecule for use according to any one of embodiments 73-76, wherein expression status of one or more genes can be used to determine whether a patient should be biopsied.
78. An RNA, cDNA or protein molecule for use according to any one of embodiments 73-76, wherein expression status of one or more genes can be used to predict disease progression in a patient.
79. An RNA, cDNA or protein molecule for use according to any one of embodiments 73-76, wherein the patient is currently undergoing or has been recommended for active surveillance.
80. An RNA, cDNA or protein molecule for use according to embodiment 78, wherein the patient is currently undergoing active surveillance by PSA monitoring, biopsy and repeat biopsy and/or MRI, at least every 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks or 24 weeks.
81. An RNA, cDNA or protein molecule for use according to any one of embodiments 73-80, wherein the method can be used to predict disease progression patients with a Gleason score of ≤10, ≤9, ≤8, ≤7 or ≤6.
82. An RNA, cDNA or protein molecule for use according to any one of embodiments 73-81, wherein the method can be used to predict:
   (i) the volume of Gleason 4 or Gleason ≥4 prostate cancer; and/or
   (ii) low risk disease that will not require treatment for 1, 2, 3, 4, 5 or more years.
83. A kit for testing for prostate cancer comprising a means for measuring the expression status of:
   (i) one or more genes selected from the group consisting of: GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion; or
   (ii) one or more genes selected from the group consisting of: EN2, ERG exons 4-5, ERG exons 6-7, GJB1, HOXC6, HPN, PCA3, PPFIA2, TMPRSS2/ERG fusion, in a biological sample, optionally wherein the kit further comprises a means for measuring serum PSA levels.
84. The kit according to embodiment 83 wherein the expression status of one or more genes is determined by methylation status, optionally wherein the expression status of one or more of GSTP1, APC, SFRP2, IGFBP3, IGFBP7 and PTGS2 is determined by methylation status.
85. The kit according to embodiment 83 wherein the expression status of one or more genes is determined by protein quantification, optionally wherein the expression status of EN2 is determined by protein quantification, further optionally wherein the expression status is determined by protein ELISA.
86. The kit according to any one of embodiments 83-85, wherein the means for detecting is a biosensor or specific binding molecule.
87. The kit according to any one of embodiments 83-86, wherein the biosensor is an electrochemical, electronic, piezoelectric, gravimetric, pyroelectric biosensor, ion channel switch, evanescent wave, surface plasmon resonance or biological biosensor
88. The kit according to any one of embodiments 83-87, wherein the means for detecting the expression status of the one or more genes is a microarray.
89. The kit according to any one of embodiments 83-87, wherein the means for detecting the expression status of the one or more genes is an ELISA.
90. The kit according to any one of embodiments 83-89, wherein the kit comprises multiple means for detecting the expression status of the one or more genes.
91. The kit according to embodiment 90 wherein the multiple means for detecting the expression status of the one or more genes is a microarray and an ELISA.
92. The kit according to embodiment 91 wherein the multiple means for detecting the expression status of the one or more genes is multiple microarrays (e.g. an expression microarray and a methylation microarray).
93. The kit according to any one of embodiments 83-92, wherein the microarray comprises specific probes that hybridise to one or more genes selected from the group consisting of: GSTP1, APC, SFRP2, IGFBP3, IGFBP7, PTGS2, ERG exons 4-5, ERG exons 6-7, GJB1, HOXC6, HPN, PCA3, SNORA20, TIMP4 and TMPRSS2/ERG fusion.
94. The kit according to any one of embodiments 83-92, wherein the microarray comprises specific probes that hybridise to one or more genes selected from the group consisting of: EN2, ERG exons 4-5, ERG exons 6-7, GJB1, HOXC6, HPN, PCA3, PPFIA2, TMPRSS2/ERG fusion.
95. The kit according to any one of embodiments 83-92, wherein the microarray comprises a probe having a nucleotide sequence with at least 80%, 85%, 90%, 95%, 96%, 97%, 98% or 99% identity to a nucleotide sequence selected from any one of SEQ ID NOs 1 to 334.
96. The kit according to any one of embodiments 83-92, wherein the microarray comprises a probe having a nucleotide sequence selected from any one of SEQ ID NOs 1 to 334.
97. The kit according to any one of embodiments 83-92, wherein the microarray comprises 334 probes each having a nucleotide sequence with at least 80%, 85%, 90%, 95%, 96%, 97%, 98% or 99% identity to a unique nucleotide sequence selected from any one of SEQ ID NOs 1 to 334.
98. The kit according to any one of embodiments 83-92, wherein the microarray comprises 334 probes, each having a unique nucleotide sequence selected from SEQ ID NOs 1 to 334.
99. The kit according to any one of embodiments 83-92, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 277 and SEQ ID NO: 278, and SEQ ID NO: 313 and SEQ ID NO: 314.
100. The kit according to embodiment 99, wherein the microarray comprises a pair of probes for every gene of interest having nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 277 and SEQ ID NO: 278, and SEQ ID NO: 313 and SEQ ID NO: 314.
101. The kit according to any one of embodiments 83-92, wherein the microarray comprises a pair of probes having a nucleotide sequence with at least 80%, 85%, 90%, 95%, 96%, 97%, 98% or 99% identity to a pair of nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 219 and SEQ ID NO: 220, SEQ ID NO: 265 and SEQ ID NO: 266, and SEQ ID NO: 317 and SEQ ID NO: 318.
102. The kit according to embodiment 101 wherein the microarray comprises a pair of probes for every gene of interest having nucleotide sequences selected from the following list: SEQ ID NO: 83 and SEQ ID NO: 84, SEQ ID NO: 87 and SEQ ID NO: 88, SEQ ID NO: 89 and SEQ ID NO: 90, SEQ ID NO: 103 and SEQ ID NO: 104, SEQ ID NO: 121 and SEQ ID NO: 122, SEQ ID NO: 123 and SEQ ID NO: 124, SEQ ID NO: 211 and SEQ ID NO: 212, SEQ ID NO: 219 and SEQ ID NO: 220, SEQ ID NO: 265 and SEQ ID NO: 266, and SEQ ID NO: 317 and SEQ ID NO: 318.
103. The kit according to any one of embodiments 83-102, wherein the kit further comprises one or more solvents for extracting RNA and/or protein from the biological sample.
104. A computer apparatus configured to perform a method according to any one of embodiments 1 to 67.
105. A computer readable medium programmed to perform a method according to any one of embodiments 1 to 67.
106. A kit of any one of embodiments 83-103, further comprising a computer readable medium as defined in embodiment 105.

### REFERENCES

[1] Cancer Research UK. Prostate cancer incidence statistics [Internet]. 2019 [cited 2019 Jun. 29]. Available from: http://www/cancerresearchuk.org/health-professional/cancer-statistics/statistics-by-cancer-type/prostate-can cer/incidence
[2] Sanda M G, Cadeddu J A, Kirkby E, Chen R C, Crispino T, Fontanarosa J, et al. Clinically Localized Prostate Cancer: AUA/ASTRO/SUO Guideline. Part I: Risk Stratification, Shared Decision Making, and Care Options. Journal of Urology [Internet]. 2018; 199(3):683-90. Available from: https://doi.org/10.1016/j.juro.2017.11.095
[3] Cornford P, Bellmunt J, Bolla M, Briers E, De Santis M, Gross T, et al. EAU-ESTRO-SIOG Guidelines on Prostate Cancer. Part II: Treatment of Relapsing, Metastatic, and Castration-Resistant Prostate Cancer. European Urology [Internet]. 2017; 71(4):630-42. Available from: https://doi.org/10.1016/j.eururo.2016.08.002.
[4] National Institute for Health and Care Excellence. Prostate cancer: diagnosis and management (update). NICE.
[5] D'Amico A V., Whittington R, Bruce Malkowicz S, et al. Biochemical outcome after radical prostatectomy, external beam radiation therapy, or interstitial radiation therapy for clinically localized prostate cancer. J Am Med Assoc. 1998; 280(11):969-974. doi:10.1001/jama.280.11.969.
[6] Epstein J I, Zelefsky M J, Sjoberg D D, et al. A Contemporary Prostate Cancer Grading System: A Validated Alternative to the Gleason Score. Eur Urol. 2016; 69(3):428-435. doi:10.1016/j.eururo.2015.06.046.
[7] Sanda M G, Cadeddu J A, Kirkby E, et al. Clinically Localized Prostate Cancer: AUA/ASTRO/SUO Guideline. Part I: Risk Stratification, Shared Decision Making, and Care Options. J Urol. 2018; 199(3):683-690. doi:10.1016/j.juro.2017.11.095.
[8] Mottet N, Bellmunt J, Bolla M, et al. EAU-ESTRO-SIOG Guidelines on Prostate Cancer. Part 1: Screening, Diagnosis, and Local Treatment with Curative Intent. Eur Urol. 2017; 71(4):618-629. doi:10.1016/j.eururo.2016.08.003.
[9] National Institute for Health and Care Excellence. Prostate Cancer: Diagnosis and Treatment.; 2014.
[10] Selvadurai E D, Singhera M, Thomas K, et al. Medium-term outcomes of active surveillance for localised prostate cancer. Eur Urol. 2013; 64(6):981-987. doi:10.1016/j.eururo.2013.02.020.
[11] Cooperberg M R, Freedland S J, Pasta D J, et al. Multiinstitutional validation of the UCSF cancer of the prostate risk assessment for prediction of recurrence after radical prostatectomy. Cancer. 2006; 107(10):2384-2391. doi:10.1002/cncr.22262.
[12] Brajtbord J S, Leapman M S, Cooperberg M R. The CAPRA Score at 10 Years: Contemporary Perspectives and Analysis of Supporting Studies. Eur Urol. 2017; 71(5):705-709. doi:10.1016/j.eururo.2016.08.065.
[13] Martin R M, Donovan J L, Turner E L, Metcalfe C, Young G J, Walsh El, et al. Effect of a low-intensity PSA-based screening intervention on prostate cancer mortality: The CAP randomized clinical trial. JAMA—Journal of the American Medical Association [Internet]. 2018 March; 319(9):883-95. Available from: http://jama.jamanetwork.com/article.aspx?doi=10.1001/jama.2018.0154
[14] Donovan J L, Hamdy F C, Lane J A, Mason M, Metcalfe C, Walsh E, et al. Patient-Reported Outcomes after Monitoring, Surgery, or Radiotherapy for Prostate Cancer. New England Journal of Medicine [Internet]. 2016 October; 375(15):1425-37. Available from: https://www.neim.org/doi/10.1055/NEJMoa1606221
[15] Ahmed H U, El-Shater Bosaily A, Brown L C, Gabe R, Kaplan R, Parmar M K, et al. Diagnostic accuracy of multi-parametric MRI and TRUS biopsy in prostate cancer (PROMIS): a paired validating confirmatory study. The Lancet [Internet]. 2017 February; 389(10071):815-22. Available from: https://www.ncbi.nim.nih.gov/pubmed/28110982
[16] Pepe P, Pennisi M. Gleason score stratification according to age at diagnosis in 1028 men. Wspolczesna Onkologia [Internet]. 2015; 19(6):471-3. Available from: https://www.ncbi.nim.nih.gov/pmc/articles/PMC4731454/pdl/WO-1926454.pdf
[17] Sonn G A, Fan R E, Ghanouni P, Wang N N, Brooks J D, Loening A M, et al. Prostate Magnetic Resonance Imaging Interpretation Varies Substantially Across Radiologists [Internet]. Elsevier; 2018. Available from: https://www.sciencedirect.com/science/articles/pii/S2405456917302663
[18] Walz J. The “PROMIS” of Magnetic Resonance Imaging Cost Effectiveness in Prostate Cancer Diagnosis?European Urology [Internet]. 2018 January; 73(1):31-2. Available from: https://www.ncbi.nim.nih.gov/pubmed/28965689.
[19] Hessels D, Klein Gunnewiek J M, Van Oort I, Karthaus H F, Van Leenders G J, Van Balken B, et al. DD3PCA3-based molecular urine analysis for the diagnosis of prostate cancer. European Urology [Internet]. 2003 July; 44(1):8-16. Available from: http://linkinghub.eisevier.com/retrieve/pii/S030228380300201X
[20] Van Neste L, Hendriks R J, Dijkstra S, Trooskens G, Cornel E B, Jannink S A, et al. Detection of High-grade Prostate Cancer Using a Urinary Molecular Biomarker-Based Risk Score. European Urology. 2016; 70(5):740-8.
[21] McKiernan J, Donovan M J, O'Neill V, Bentink S, Noerholm M, Belzer S, et al. A novel urine exosome gene expression assay to predict high-grade prostate cancer at initial biopsy. JAMA Oncology [Internet]. 2016 July; 2(7):882-9. Available from: http://oncology.jamanetwork.com/articles.aspx?doi=10.1001/jamaoncol.2016.0097
[22] Zhao F, Olkhov-Mitsel E, Kamdar S, Jeyapala R, Garcia J, Hurst R, et al. A urine-based DNA methylation assay, ProCUrE, to identify clinically significant prostate cancer. Clinical Epigenetics [Internet]. 2018 December; 10(1):147. Available from: https://clinicalpigeneticsjournal.biomedcentral.com/articles/10.1186/s13148-018-0575-z
[23] Brikun I, Nusskern D, Decatus A, Harvey E, Li L, Freije D. A panel of DNA methylation markers for the detection of prostate cancer from FV and DRE urine DNA. Clinical Epigenetics [Internet]. 2018; 10(1). Available from: https://www.doi.org/10.1186/s13148-018-0524-x
[24] Luca B A, Brewer D S, Edwards D R, Edwards S, Whitaker H C, Merson S, et al. DESNT: A Poor Prognosis Category of Human Prostate Cancer [Internet]. Elsevier; 2017. Available from: https://www.sciencedirect.com/science/article/pii/S2405456917300251
[25] Knezevic D, Goddard A D, Natraj N, Cherbavaz D B, Clark-Langone K M, Snable J, et al. Analytical validation of the Oncotype D X prostate cancer assay—a clinical RT-PCR assay optimized for prostate needle biopsies. BMC Genomics. 2013 October; 14(1):690.
[26] Cuzick J, Berney D M, Fisher G, Mesher D, Møller H, Reid J E, et al. Prognostic value of a cell cycle progression signature for prostate cancer death in a conservatively managed needle biopsy cohort. British Journal of Cancer. 2012 March; 106(6):1095-9.
[27] Eklund M, Nordstrbm T, Aly M, Adolfsson J, Wiklund P, Brandberg Y, et al. The Stockholm-3 (STHLM3) Model can Improve Prostate Cancer Diagnostics in Men Aged 50-69 yr Compared with Current Prostate Cancer Testing. European Urology Focus. 2016; 3:4-7.
[28] Tosoian J J, Carter H B, Lepor A, Loeb S. Active surveillance for prostate cancer: Current evidence and contemporary state of practice [Internet]. Vol. 13. Nature Publishing Group; 2016. pp. 205-15. Available from: https://www.nature.com/articles/nrurol.2016.45
[29] Loeb S, Bjurlin M A, Nicholson J, Tammela T L, Penson D F, Carter H B, et al. Overdiagnosis and overtreatment of prostate cancer [Internet]. Vol. 65. Elsevier; 2014. pp. 1046-55. Available from: https://www.sciencedirect.com/science/article/piiS0302283813014905?via%3Dihub
[30] O'Reilly E, Tuzova A V, Walsh A L, Russell N M, O'Brien O, Kelly S, et al. epiCaPture: A Urine DNA Methylation Test for Early Detection of Aggressive Prostate Cancer. JCO Precision Oncology [Internet]. 2019 January; (3):1-18. Available from: http://ascopubs.org/doi/10.1200/PO.18.00134
[31] Connell S P and, Hanna M, McCarthy F, Hurst R, Webb M, Curley H, et al. A Four-Group Urine Risk Classifier for Predicting Outcome in Prostate Cancer Patients. BJU International [Internet]. 2019 May; Available from: http://doi.wiliey.com/10.111/biu.14811
[32] Stark J R, Perner S, Stampfer M J, Sinnott J A, Finn S, Eisenstein A S, et al. Gleason score and lethal prostate cancer: Does 3+4=4+3≥ Journal of Clinical Oncology. 2009; 27(21):3459-64.
[33] Collins G S, Reitsma J B, Altman D G, Moons K G. Transparent reporting of a multivariable prediction model for individual prognosis or diagnosis (tripod): The tripod statement. European Urology [Internet]. 2015; 67(6):1142-51. Available from: https://www.sciencedirect.com/science/article/pii/S0302283814011993
[34] Rak J. Microparticles in cancer. Semin Thromb Hemost 2010 November; 36(8):888-906.
[35] Mathivanan S, Ji H, Simpson R J. Exosomes: Extracellular organelles important in intercellular communication. Journal of Proteomics. Elsevier B. V; 2010 Sep. 10; 73(10):1907-20.
[36] van der Pol E, Boing A N, Harrison P, Sturk A, Nieuwland R. Classification, Functions, and Clinical Relevance of Extracellular Vesicles. Pharmacological Reviews. 2012 Jul. 2; 64(3):676-705.
[37] Keller S, Sanderson M P, Stoeck A, Altevogt P. Exosomes: from biogenesis and secretion to biological function. Immunol Lett 2006 Nov. 15; 107(2):102-8.
[38] Simons M, Raposo G. Exosomes—vesicular carriers for intercellular communication. Current Opinion in Cell Biology. 2009 August; 21(4):575-81.
[39] van Niel G. Exosomes: A Common Pathway for a Specialized Function. Journal of Biochemistry. 2006 Jul. 1; 140(1):13-21.
[40] Miranda K C, Bond D T, McKee M, et al. Nucleic acids within urinary exosomes/microvesicles are potential biomarkers for renal disease. Kidney Int. 2010; 78(2):191-199. doi:10.1038/ki.2010.106.
[41] Mears R, Craven R A, Hanrahan S, Totty N. Proteomic analysis of melanoma-derived exosomes by two-dimensional polyacrylamide gel electrophoresis and mass spectrometry. Proteomics 2004 December; 4(12):4019-31.
[42] Futter C E, White I J. Annexins and endocytosis. Traffic 2007 August; 8(8):951-8.
[43] Xiao D, Ohlendorf J, Chen Y, Taylor D D, Rai S N, Waigel S, et al. Identifying mRNA, microRNA and protein profiles of melanoma exosomes. PLoS ONE. 2012; 7(10):e46874.
[44] Wieckowski E, Whiteside T L. Human tumour-derived vs dendritic cell-derived exosomes have distinct biologic roles and molecular profiles. Immunol Res. 2006; 36(1-3):247-54.
[45] Castellana D, Zobairi F, Martinez M C, Panaro M A, Mitolo V, Freyssinet J-M, et al. Membrane microvesicles as actors in the establishment of a favorable prostatic tumoural niche: a role for activated fibroblasts and CX3CL1-CX3CR1 axis. Cancer Research. 2009 Feb. 1; 69(3):785-93.
[46] Mitchell P J, Welton J, Staffurth J, Court J, Mason M D, Tabi Z, et al. Can urinary exosomes act as treatment response markers in prostate cancer≥ J Transl Med. 2009; 7(1):4.
[47] Schostak M, Schwall G P, Poznanovid S, Groebe K, Müller M, Messinger D, et al. Annexin A3 in Urine: A Highly Specific Noninvasive Marker for Prostate Cancer Early Detection. The Journal of Urology. 2009 January; 181(1):343-53.
[48] Nilsson J, Skog J, Nordstrand A, Baranov V, Mincheva-Nilsson L, Breakefield X O, et al. Prostate cancer-derived urine exosomes: a novel approach to biomarkers for prostate cancer. Nature Publishing Group; 2009 Apr. 28; 100(10):1603-7.
[49] Fitzwater & Polisky (1996) Methods Enzymol, 267:275-301
[50] Vickers A J, Elkin E B. Decision Curve Analysis: A Novel Method for Evaluating Prediction Models. Med Decis Mak. 2006; 26(6):565-574. doi:10.1177/0272989X06295361.
[51] R Core Team. R: A Language and Environment for Statistical Computing [Internet]. Vienna, Austria: R Foundation for Statistical Computing; 2019. Available from: http://www.r-project.org/
[52] Lane J A, Donovan J L, Davis M, Walsh E, Dedman D, Down L, et al. Active monitoring, radical prostatectomy, or radiotherapy for localised prostate cancer: Study design and diagnostic and baseline results of the ProtecT randomised phase 3 trial. The Lancet Oncology [Internet]. 2014 September; 15(10):1109-18.
[53] Ciccarese C, Massari F, lacovelli R, Fiorentino M, Montironi R, Nunno V D, et al. Prostate cancer heterogeneity: Discovering novel molecular targets for therapy. Cancer Treatment Reviews [Internet]. 2017; 54:68-73.
[54] Xia Y, Huang C-C, Dittmar R, Du M, Wang Y, Liu H, et al. Copy number variations in urine cell free DNA as biomarkers in advanced prostate cancer. Oncotarget [Internet]. 2016 June; 7(24):35818-31.
[55] Killick E, Morgan R, Launchbury F, Bancroft E, Page E, Castro E, et al. Role of Engrailed-2 (EN2) as a prostate cancer detection biomarker in genetically high risk men. Scientific Reports [Internet]. 2013; 3:2059.
[56] Strand S H, Bavafaye-Haghighi E, Kristensen H, Rasmussen A K, Hoyer S, Borre M, et al. A novel combined miRNA and methylation marker panel (miMe) for prediction of prostate cancer outcome after radical prostatectomy. International Journal of Cancer [Internet]. 2019 June; ijc.32427.
[57] Tomlins S A, Day J R, Lonigro R J, Hovelson D H, Siddiqui J, Kunju L P, et al. Urine TMPRSS2:ERG Plus PCA3 for Individualized Prostate Cancer Risk Assessment. European Urology [Internet]. 2016; 70(1):45-53.
[58] Ricketts C J, De Cubas A A, Fan H, Smith C C, Lang M, Reznik E, et al. The Cancer Genome Atlas Comprehensive Molecular Characterization of Renal Cell Carcinoma. Cell Reports [Internet]. 2018; 23(1):313-326.e5.
[59] The Human Protein Atlas. Expression of GJB1 in cancer [Internet]. [cited 2019 May 24]. Available from: https://www.proteinatlas.org/ENSG00000169562-GJB1/pathology
[60] Tomlins S A, Laxman B, Varambally S, Cao X, Yu J, Helgeson B E, et al. Role of the TMPRSS2-ERG gene fusion in prostate cancer. Neoplasia (New York, NY) [Internet]. 2008 February; 10(2):177-88.
[61] Guyon I, Elisseeff A. An introduction to variable and feature selection. Journal of machine learning research. 2003; 3(March):1157-82.
[62] Kursa M B, Rudnicki W R. Feature Selection with the Boruta Package. Journal of Statistical Software. 2010; 36(11).
[63] Breiman L. Random forests. Machine Learning [Internet]. 2001; 45(1):5-32. Available from: http://link.springer.com/10.1023/A:1010933404324
[64] Liaw A, Wiener M. Classification and regression by randomForest. R News [Internet]. 2002; 2(3):18-22. Available from: https://CRAN.R-project.org/doc/Rnews/
[65] Robin X, Turck N, Hainard A, Tiberti N, Lisacek F, Sanchez J-C, et al. PROC: An open-source package for r and s+ to analyze and compare roc curves. BMC Bioinformatics. 2011; 12:77.
[66] Wickham H. Ggplot2: Elegant graphics for data analysis [Internet]. Springer-Verlag New York; 2016. Available from: https://ggplot2.tidyverse.org
[67] Ho J, Tumkaya T, Aryal S, Choi H, Claridge-Chang A. Moving beyond P values: data analysis with estimation graphics. Nature Methods [Internet]. 2019 June; 1. Available from: http://www.nature.com/articles/s41592-019-0470-3
[68] Vickers A J, Elkin E B. Decision Curve Analysis: A Novel Method for Evaluating Prediction Models. Medical Decision Making [Internet]. 2006; 26(6):565-74. Available from: http://journals.sagepub.com/doi/10.1177/0272989X06295361
[69] Brown M. rmda: Risk Model Decision Analysis [Internet]. 2018. Available from: https://cran.r-project.org/package=rmda
[70] Kerr K F, Brown M D, Zhu K, Janes H. Assessing the clinical impact of risk prediction models with decision curves: Guidance for correct interpretation and appropriate use. Journal of Clinical Oncology [Internet]. 2016; 34(21):2534-40. Available from: www.jco.org
[71] Geiss G K, Bumgarner R E, Birditt B, et al. Direct multiplexed measurement of gene expression with color-coded probe pairs. Nat Biotechnol. 2008; 26(3):317-325. doi:10.1038/nbt1385.
[72] Morgan R, Boxall A, Bhatt A, Bailey M, Hindley R, Langley S, et al. Engrailed-2 (EN2): A tumor specific urinary biomarker for the early diagnosis of prostate cancer. Clinical Cancer Research [Internet]. 2011 March; 17(5):1090-8. Available from: http://www.ncbi.nlm.nih.gov/pubmed/21364037 http://clincancerres.aacrjournals.org/cgi/doi/10.1158/1078-0432.CCR-10-2410

1-16. (canceled)

17. A method of diagnosing or testing for prostate cancer in a subject comprising determining the methylation status of one or more genes selected from the group consisting of GSTP1, APC, SFRP2, IGFBP3, IGFBP7, PTGS2, and the expression status of one or more genes selected from the group consisting of ERG exons 4-5, ERG exons 6-7, GJB1, HOXC6, HPN, PCA3, SNORA20, TIMP4 and TMPRSS2/ERG fusion in a biological sample from the subject.

18-20. (canceled)

21. The method according to claim 17, wherein the method can be used to determine whether a patient should be biopsied.

22. The method according to claim 17, wherein the biological sample is processed prior to determining the expression status of the one or more genes in the biological sample.

23. The method according to claim 17, wherein determining the expression status of the one or more genes comprises extracting RNA from the biological sample, optionally wherein the RNA is extracted from extracellular vesicles.

24. The method according to claim 17, wherein the biological sample is a urine sample.

25. The method according to claim 17, wherein the sample is from a human.

26. The method according to claim 17, wherein the serum PSA level of the subject is also used in the method of diagnosing or testing for prostate cancer.

27. The method according to claim 17, wherein the expression status of one or more genes is determined by one or more methods including protein quantification, methylation status, RNA extraction, RNA hybridization, or RNA sequencing.

28. The method of claim 21, wherein the method is used in combination with MRI imaging data to determine whether a patient should be biopsied.

29. The method according to claim 28, wherein the MRI imaging data is generated using multiparametic-MRI (MP-MRI).

30. The method according to claim 17, wherein the patient is currently undergoing or has been recommended for active surveillance.

31. The method according to claim 17, wherein the method can be used to predict disease progression in patients with a Gleason core of ≤10, ≤9, ≤8, ≤7, or ≤6.

32. The method according to claim 17, wherein the method can be used to predict:
(i) the volume of Gleason 4 or Gleason ≥4 prostate cancer; and/or
(ii) low risk disease that will not require treatment for 1, 2, 3, 4, 5, or more years.

33. The method according to claim 17, wherein determining the expression status of the one or more genes comprises the step of quantifying the expression status of the RNA transcript or cDNA molecule and wherein the expression status of the RNA or cDNA is quantified using any one or more of the following techniques: microarray analysis, real-time quantitative PCR, DNA sequencing, RNA sequencing, NanoString®, Norther blot analysis, in situ hybridization, and detection and quantification of a binding molecule.

34. A method of treating prostate cancer, comprising diagnosing a patient as having or as being suspected of having prostate cancer using a method as defined in claim 17 and administering to the patient a therapy for treating prostate cancer.

35. The method according to claim 34, wherein the therapy for prostate cancer comprises surgery, brachytherapy, active surveillance, chemotherapy, hormone therapy, immunotherapy, and/or radiotherapy.

36. The method according to claim 35, wherein the chemotherapy comprises administration of one or more agents selected from the following list: abiraterone acetate, apalutamide, bicalutamide, cabazitaxel, degarelix, docetaxel, leuprolide acetate, enzalutamide, flutamide, goserelin acetate, mitoxantrone, nilutamide, sipuleucel T, and radium 223 dichloride.

37. The method of claim 34, wherein the therapy for prostate cancer comprises resection of all or part of the prostate gland or resection of a prostate tumor.

38. A method of treating prostate cancer, wherein the patient has been determined as having prostate cancer or as being suspected of having prostate cancer using a method as defined in claim 1, comprising administering to the patient a therapy for treating prostate cancer.

39. The method according to claim 38, wherein the therapy for prostate cancer comprises surgery, brachytherapy, active surveillance, chemotherapy, hormone therapy, immunotherapy, and/or radiotherapy.

40. The method according to claim 39, wherein the chemotherapy comprises administration of one or more agents selected from the following list: abiraterone acetate, apalutamide, bicalutamide, cabazitaxel, degarelix, docetaxel, leuprolide acetate, enzalutamide, flutamide, goserelin acetate, mitoxantrone, nilutamide, sipuleucel T, and radium 223 dichloride.

41. The method of claim 40, wherein the therapy for prostate cancer comprises resection of all or part of the prostate gland or resection of a prostate tumor.
